SEQUENCE LISTING (488 sequences)

SEQ ID NO: 1 - DNA, 4532 bp, Homo sapiens
agtgacttgagggaggcgctgcgactgacaagcggctctgcccgggaccttctcgctttcatctagcgctgcactcaatggaggggcgggcaccgcagtgcttaatgctgtcttaactagtgtaggaaaacggctcaacccaccgctgccgaaatgaagtataagaatcttatggcaagggccttatatgacaatgtcccagagtgtgccgaggaactggcctttcgcaagggagacatcctgaccgtcatagagcagaacacagggggactggaaggatggtggctgtgctcgttacacggtcggcaaggcattgtcccaggcaaccgggtgaagcttctgattggtcccatgcaggagactgcctccagtcacgagcagcctgcctctggactgatgcagcagacctttggccaacagaagctctatcaagtgccaaacccacaggctgctccccgagacaccatctaccaagtgccaccttcctaccaaaatcagggaatttaccaagtccccactggccacggcacccaagaacaagaggtatatcaggtgccaccatcagtgcagagaagcattgggggaaccagtgggccccacgtgggtaaaaaggtgataacccccgtgaggacaggccatggctacgtatacgagtacccatccagataccaaaaggacgtctatgatatccctccttctcataccactcaaggggtatacgacatccctccctcatcagcaaaaggccctgtgttttcagttccagtgggagagataaaacctcaaggggtgtatgacatcccgcctacaaaaggggtatatgccattccgccctctgcttgccgggatgaagcagggcttagggaaaaagactatgacttcccccctcccatgagacaagctggaaggccggacctcagaccggagggggtttatgacattcctccaacctgcaccaagccagcagggaaggaccttcatgtaaaatacaactgtgacattccaggagctgcagaaccggtggctcgaaggcaccagagcctgtccccgaatcacccacccccgcaactcggacagtcagtgggctctcagaacgacgcatatgatgtcccccgaggcgttcagtttcttgagccaccagcagaaaccagtgagaaagcaaacccccaggaaagggatggtgtttatgatgtccctctgcataacccgccagatgctaaaggctctcgggacttggtggatgggatcaaccgattgtctttctccagtacaggcagcacccggagtaacatgtccacgtcttccacctcctccaaggagtcctcactgtcagcctccccagctcaggacaaaaggctcttcctggatccagacacagctattgagagacttcagcggctccagcaggcccttgagatgggtgtctccagcctaatggcactggtcactaccgactggcggtgttacggatatatggaaagacacatcaatgaaatacgcacagcagtggacaaggtggagctgttcctgaaggagtacctccactttgtcaagggagctgttgcaaatgctgcctgcctcccggaactcatcctccacaacaagatgaagcgggagctgcaacgagttgaagactcccaccagatcctgagtcaaaccagccatgacttaaatgagtgcagctggtccctgaatatcttggccatcaacaagccccagaacaagtgtgacgatctggaccggtttgtgatggtggcaaagacggtgcccgatgacgccaagcagctcaccacaaccatcaacaccaacgcagaggccctcttcagacccggccctggcagcttgcatctgaagaatgggccggagagcatcatgaactcaacggagtacccacacggtggctcccagggacagctgctgcatcctggtgaccacaaggcccaggcccacaacaaggcactgcccccaggcctgagcaaggagcaggcccctgactgtagcagcagtgatggttctgagaggagctggatggatgactacgattacgtccacctacagggtaaggaggagtttgagaggcaacagaaagagctattggaaaaagagaatatcatgaaacagaacaagatgcagctggaacatcatcagctgagccagttccagctgttggaacaagagattacaaagcccgtggagaatgacatctcgaagtggaagccctctcagagcctacccaccacaaacagtggcgtgagtgctcaggatcggcagttgctgtgcttctactatgaccaatgtgagacccatttcatttcccttctcaacgccattgacgcactcttcagttgtgtcagctcagcccagcccccgcgaatcttcgtggcacacagcaagtttgtcatcctcagtgcacacaaactggtgttcattggagacacgctgacacggcaggtgactgcccaggacattcgcaacaaagtcatgaactccagcaaccagctctgcgagcagctcaagaccatagtcatggcaaccaagatggccgccctccattaccccagcaccacggccctgcaggaaatggtgcaccaagtgacagacctttctagaaatgcccagctgttcaagcgctctttgctggagatggcaacgttctgagaagaaaaaaaagaggaaggggactgcgttaacggttactaaggaaaactggaaatactgtctggtttttgtaaatgttatctatttttgtagatattttatataaaaatgaaatattttaacattttatgggtcagtcaactttcagaaattcagggagctggagagggaaatctttttttttccccctgagtggttcttatgtacatagaggtatctgagacataaactgtacagaaaacttgtccacgtgcttttgtatgcccatgtattcatgtttgtttgtagatgtttgtctgatgcatttcattaaaaaaaaaaccatgaattacgaagcaccttagtaagcacctcctaatgctgcattttttttgttgttgttaaaaacataccagctggttataatattgttctccacgtccttgtgatgattctgagcctggcactcccaaatctgggaagcatagtttatttgcaagtgttcaccttccaaatcatgaggcatagcatgacttattcttgtttggaaaactcttttcaaaactgaccatcttaaacacatgatggccaagtgcccaaaagccctcttgcggagcaaatttcagaatatatatgtggatccaagctctgatagttcaggtgctggagggaagagagacctgtgtgtttagaggccaggaccacagttaggattgggttgtttcaatactgagagacagctacaataaaaggagagcaattgcctccctggggctgttcaatcttctgcatttgtgagtggttcagtcatgaggttttccaaaagatgtttttagagttgtaaaaaccatatttgcagcaaagatttacaaaggcgtatcagactatgattgttcaccaaaataggggaatggtttgatccgccagttgcaagtagaggcctttctgactcttaatattcactttggtgctactacccccattacctgagggaaactggccaggtccttgatcatggaactatagagctaccaggacatatcctgctctctaagggaatttattgctatcttgcaccttctttaaaactcacatatgcagacctgacactcaagagtggctagctacacagagtccatctaatttttgcaacttcctgtggccagtgtgtataaccccttccactatctcacagatagtcacagcgtccattccatagtctgtctcctcacatctgttagtattgacacagcacagacaccacaagccatcaggttcttcatggggcaggtgaaatacttctaccccatgggtaaatgtattcacatattaccaagagaagaagcacattatctatgatcttttggcccagttcttatttagcatttttattccagcctacttggaaacatgtttttatttgcaatatatgcctgactgaattaagcttgcttgttttaaacaaccaaatcattggaacagaaaaggatttaaaaaacaagaatgcatgatctcagagtgattaaaaaaaaatcagtggaaataaatgatcatagaaggtgcttttcaaaacaactgctattataattctcaaagtcctactctgccaaaagaagattaaaagtcatacattacattacaaggaaatgttcatgtgggaagagggttgctgaaaatcaacaacgcttgaagttaaaaagtgtgtctttgtagatttcattgtataatgtgtatttcttaggagatggctgacttgattgatctacgctaagtggagacatttcacatttttaaaaccaaatgttcaatctgtattactctttgccgtcttgtatgtagaggctatttttaaatcattaaatttttagatctctgttttcaaaaaaaaaaaaaaa

SEQ ID NO: 2 - protein, 834 aa, Homo sapiens
MKYKNLMARALYDNVPECAEELAFRKGDILTVIEQNTGGLEGWWLCSLHGRQGIVPGNRVKLLIGPMQETASSHEQPASGLMQQTFGQQKLYQVPNPQAAPRDTIYQVPPSYQNQGIYQVPTGHGTQEQEVYQVPPSVQRSIGGTSGPHVGKKVITPVRTGHGYVYEYPSRYQKDVYDIPPSHTTQGVYDIPPSSAKGPVFSVPVGEIKPQGVYDIPPTKGVYAIPPSACRDEAGLREKDYDFPPPMRQAGRPDLRPEGVYDIPPTCTKPAGKDLHVKYNCDIPGAAEPVARRHQSLSPNHPPPQLGQSVGSQNDAYDVPRGVQFLEPPAETSEKANPQERDGVYDVPLHNPPDAKGSRDLVDGINRLSFSSTGSTRSNMSTSSTSSKESSLSASPAQDKRLFLDPDTAIERLQRLQQALEMGVSSLMALVTTDWRCYGYMERHINEIRTAVDKVELFLKEYLHFVKGAVANAACLPELILHNKMKRELQRVEDSHQILSQTSHDLNECSWSLNILAINKPQNKCDDLDRFVMVAKTVPDDAKQLTTTINTNAEALFRPGPGSLHLKNGPESIMNSTEYPHGGSQGQLLHPGDHKAQAHNKALPPGLSKEQAPDCSSSDGSERSWMDDYDYVHLQGKEEFERQQKELLEKENIMKQNKMQLEHHQLSQFQLLEQEITKPVENDISKWKPSQSLPTTNSGVSAQDRQLLCFYYDQCETHFISLLNAIDALFSCVSSAQPPRIFVAHSKFVILSAHKLVFIGDTLTRQVTAQDIRNKVMNSSNQLCEQLKTIVMATKMAALHYPSTTALQEMVHQVTDLSRNAQLFKRSLLEMATF

SEQ ID NO: 3 - DNA, 1300 bp, Homo sapiens
gcgctgcgactgacaagcggctctgcccgggaccttctcgctttcatctagcgctgcactcaatggaggggcgggcaccgcagtgcttaatgctgtcttaactagtgtaggaaaacggctcaacccaccgctgccgaaatgaagtataagaatcttatggcaagggccttatatgacaatgtcccagagtgtgccgaggaactggcctttcgcaagggagacatcctgaccgtcatagagcagaacacagggggactggaaggatggtggctgtgctcgttacacggtcggcaaggcattgtcccaggcaaccgggtgaagcttctgattggtcccatgcaggagactgcctccagtcacgagcagcctgcctctggactgatgcagcagacctttggccaacagaagctctatcaagtgccaaacccacaggctgctccccgagacaccatctaccaagtgccaccttcctaccaaaatcagggaatttaccaagtccccactggccacggcacccaagaacaagaggtatatcaggtgccaccatcagtgcagagaagcattgggggaaccagtgggccccacgtgggtaaaaaggtgttccagagagatgggcaagtgtcctatttcttagtgagagcctctaaacaaaccagcttgtgaacctccactgaaaagatctcatctgatgagcattttaataaagtgtcctgagtttggaggcttgccgtctttctcttggataaatatcttcatctcctagacttggaaaaacacattttctcctggggtttcccattggcgtgtcttgagctgctctggtgataaccgtaataatgccaatactgatacgaacagcagaaaacagtaaccccaagaactctacagatgatcatcaaggaccactgtctcttaccatttgctgctttggtttgaaattctcactgcctcgtagatctcattttgagcactatacattcctaaagattgatttctttctatctgacttaaatttaggaatgattaaatcttcatttctcccatgatttgatcctaaaacattttgaaaggaaacagccttgagatctgtgattactaagacatacataacattcttatcacattagaaagcaagaattgactgttgcttgtcttgttcctgttgtcttgtcccctgaattcctgtttatctttgattgtatgtgggacattgtattttcagtacatttgtagaaataatgtgaagcctataaagatgttctctgcctccaaaaaaaaaaaaaaaaaaaaaaaaaaaaaa

SEQ ID NO: 4 - protein, 174 aa, Homo sapiens
MKYKNLMARALYDNVPECAEELAFRKGDILTVIEQNTGGLEGWWLCSLHGRQGIVPGNRVKLLIGPMQETASSHEQPASGLMQQTFGQQKLYQVPNPQAAPRDTIYQVPPSYQNQGIYQVPTGHGTQEQEVYQVPPSVQRSIGGTSGPHVGKKVFQRDGQVSYFLVRASKQTSL

SEQ ID NO: 5 - DNA, 5913 bp, Homo sapiens
gtggacgcagcgggctttggaaaggccccaagttaatgaggcgtgcgccggctgccgagcgcctcttggagctgggctttcccccgcggtgcgggcgccaggagccgccttttccgctgggtgtcactcgggggtggggaagatggcccattcaaaagcgccgcgagggggcccggccagtgcccttcagtgagcgctcgcaagaggacggcagaggcccggcagctcggagctccgggaccttgtggcgcatcaggacgcggctgtccctctgccgggacccagagccgccgccgccgctctgcctcctgcgtgttagcctcctctgcgcgctccgggcaggcggccgtgggagccgctggggcgaggacggcgcgaggctgctgctgctgcccccggcccgcgcggctggaaacggagaggccgagccaagcggcggcccctcttatgctgggaggatgctggagagtagcggctgcaaagcgctgaaggagggcgtgctggagaagcgcagcgacgggttgttgcagctctggaagaaaaagtgttgcatcctcaccgaggaagggctgctgcttatcccgcccaagcagctgcaacaccagcagcagcagcagcaacagcagcagcagcagcagcaacaacagcccgggcaggggccggccgagccgtcccaacccagtggccccgctgtcgccagcctcgagccgccggtcaagctcaaggaactgcacttctccaacatgaagaccgtggactgtgtggagcgcaagggcaagtacatgtacttcactgtggtgatggcagagggcaaggagatcgactttcggtgcccgcaagaccagggctggaacgccgagatcacgctgcagatggtgcagtacaagaatcgtcaggccatcctggcggtcaaatccacgcggcagaagcagcagcacctggtccagcagcagcccccctcgcagccgcagccgcagccgcagctccagccccaaccccagcctcagcctcagccgcaaccccagccccaatcacaaccccagcctcagccccaacccaagcctcagccccagcagctccacccgtatccgcatccacatccacatccacactctcatcctcactcgcacccacaccctcacccgcacccgcatccgcaccaaataccgcacccacacccacagccgcactcgcagccgcacgggcaccggcttctccgcagcacctccaactctgcctgaaaggggcagctcccgggcaagacaaggttttgaggacttgaggaagtgggacgagcacatttctattgtcttcacttggatcaaaagcaaaacagtctctccgccccgcaccagatcaagtagtttggacatcaccctactgaaaacttgcgattcttcttagttttctgcatacttttcatcacgatgcaggaaacgatttcgagtcaagaagacttttatttatgaacctttgaaaggatcgtcttgtatggtgaattttctaggagcgatgatgtactgtaattttattttaatgtattttgatttatgattatttattagtttttttttaaatgcttgttctaagacatttctgaatgtagaccattttccaaaaaggaaactttattttcaaaaacctaatccgtagtaattcctaatcttggagaataaaaaagggcggtggaggggaaaacattaagaatttattcattatttctcgagtactttcagaaagtctgacactttcattgttgtgccagctggttgaaattaaaactctgatattactttttttgaggatttttatttttgtttttgcttaaacatatagtttgtctagaagtttaaaaagctaaaagttaaaaatggtgtaattatgaaaatctaacactcaagatagtttctaaaaggaaatcagtagttaaggatacctgatttcaaaatatttaaagcataacctaactgatggtaggatgattgtatcttgaatatgtggtagggccacatctattgtaggaaaaccttgcttttatcatctgtgtgtaaagggcttaataaggagaagaggccttttgactgatttgtgagtataaatgcatttgctgtttcatttcaaaaatgttgtggaggaaaagagtacatttaacttgtataagagaatatttgtactcctgtccaggctgcaggacctttcttcgagagctttgcacacttgacttgaaccacattttctgatccctttactttgttttagaagcacactgaaaaatctcgttgtttaaagtacaatttgtaaatatttcaaaggtctaggagtcataacttttgttttcatactgaaaatgatgttgatcagagaaaccaactgttttgcttttcattgctctgtgagaaatttgaggattctgttttgctgttaggtaagctaaactcagaaattgaaaaggaaaagactggataaacacaggattttcagtaagaaaacaaccccagtcttgtcttagaagccacttgttgaggagtctgttgggggaaaaaagaggatatgcttttaaaggtagaacaaaccttcttctgtgttaaatcaaaaggatgttcaaaatccaccaggacagatgctacttgggtttaaatggagccatagatgatacaaagtcctcttggggctgaaaatcacttcctatttgcatggctttactaactggtttctgttttccattatctttttcacagaaagtcttggtcagtatttttccagcatttaaattgaaacggtcagtattagaccactgctaggttatgtagtcaagaaataaaaatagaattacatgctacagatgtctttattctccttccatctagaaaggagttccaaggtcaaattactttttagtgcaatagttaaatgacattttgagatcataactcatatccaaaaagttgcagggaaaattaaaatagctttcccctattaagctaatggcaaacaaaacttaagtggacccccacttccagtggttgtttaggttgcagttgtgaaaatatgctgccaacatttaaaaacttgtttcatatgtatatatgtatacacatatatgaatatgtatgtatatatacatatatgagaacatgtgtgtacacatatatgaatatgtatatatgtgtatgtatgtatatatgtatatgaaatgagagccacatctaaagatttcttaaatcaagtttggttcagcttccttagaactgtggctgtactttttgaggagtacctcatagtactatatttttaatgcatgcaaatcataatagctccaaatgaaccacagttttttcccaatggaggatttttttttaattcttgtactaaaaaaaaaaaatccataccaaatatttttacaaattaagattgatgtaggttttaaaaaaggcatttgtatgttgttagcttacatatggggctaggtaatttcattgcttaaaaagatgcgcctaggctccctcttggtggctggatttctttttcttcgcccgtggtggccatggttcttaatagggccaccggaatcatggtttctttttttttttttttttttgagatggagtctcgccctgtgacccaggctggagtgcagtggcacgatctcggctcactgcaacctctgcctcctgggttcacgccattctcctgtctcagcctcctgagtagctgggactacaggtgaatgccaccacgcccggctgatttttgtatttttagtagagatggggtttcaccatagtggtcaggctgttctcgaactcctgacctcaggtgatccacctgccttggcctcccaaagtgctaggattacaggtgtgagccaccacacccggccccagaataatggtttcttgactttctgtagcccttgttccttagtctgctgtgatatttatgttgacctttatcattttctattctgaacccctcttagcatttaatgtgaaatctaagaaattagaagtagaatggcttttattgttttgacacctttgaaattattattaataatttttccagagcaaaaaagcaaacacgctcaataagactaaacaaaacaaaatataaatgtacatcatttaatgtcccagtggctctattctacctgtaagaaaatgatacaaaaccacctaagatattttgaagcctgacaaatcagcttcatggaaaaaggtaaaaaatgcatttttcaaccgaaagggcagatccaatagaagacccgctccttaaataaacataaaatgtaaaaagttggaaaattaagagtaatgttccatctggaaactgaacttttgtccttgaacttgtgttggcaccaagcctcatacacagtgagctcaataactgttgggacaaaggaaggaaggacaaaatgtgtaacttcccagcatctgggagatgctgtctcttgcctcactgagtgttccttttctttgctctcatgtcattccctgagaacaatgaattctgggacaggctaaacatcatgatgaagtttcttaaacagactttcttagtggaaatccatttagatctgggtgtgctctatggggagtgctgacgtcaaagagcaaatgtctataaggggcccttttaaaatgaacattttcctcattgagcaagctgggattctctaatgtagaaatcaagccatctttataatttcacttcagatgtttatgtttttgttttttttgtctccaatgatggtaaaaataaaaactacgcattacttaaaggagtttccctcacatgtaaacactgttaggaagtctggattaagttgaaagtcctgttttaactttttttctctcatataccaaacactctgtatttctcttaaagaagccctttaagagaaagccctaattttatatctgacagtaaagtttgctgcaagtgtatgagttcaaacacatcccttgttttctgtccctaggggaaaagtcatgtagttttagcttggctccagtgttaatattatattcagtagcagccttagaagagtggtctaagacttgaacctggagcaattttatagcacagaatcctacgaagataggactgtgaacatttgttttctttttcgtgtgtgtcaaactaactggtttttgctttaccaataaaatgtcctcggcagagtaaattttaaacgtgaaaattatagatcttgatattgaatccatcagtgattcaagagatacacctatttgcctaaaacaacctaagatgtattggttatggaatcatgtgttggataggttcttaagacctgtttcctcaaatcttgacacagttttcaagggtggcttattgacttgcacggttgggcagataatccagatttacctaagattgggtaaaaaagtcatctgtgactttgctggcagggcatttgctaagtggagtacaggatctaaaagggttttcttagaaagggcaatattgtccaatgaagtaagcagaaggactctgggttagaagcatctgcacaaaaactggtgagacctactctccactgctctgcagctggatggctgatggcaggctgagcagtggggaagcaggttttaacaacagggagtccttccaggtcactgtatattgagaagaaacataaaactattgtctgttacattccgaggtcagccttcttcttaacgttttataatatgcaaatgccagcttctggaaagcaagtatcatcatgtaccaaatgctttatacaccatcacattcatgaattttttagcatggtcagaacttgtgtaaatatgtctcttagatgattttggggagatgtgatttatttttcatattttcaaaatgcatttcatttcaaataaagttatctattgagacaaccga

SEQ ID NO: 6 - protein, 401 aa, Homo sapiens
MRRAPAAERLLELGFPPRCGRQEPPFPLGVTRGWGRWPIQKRREGARPVPFSERSQEDGRGPAARSSGTLWRIRTRLSLCRDPEPPPPLCLLRVSLLCALRAGGRGSRWGEDGARLLLLPPARAAGNGEAEPSGGPSYAGRMLESSGCKALKEGVLEKRSDGLLQLWKKKCCILTEEGLLLIPPKQLQHQQQQQQQQQQQQQQQPGQGPAEPSQPSGPAVASLEPPVKLKELHFSNMKTVDCVERKGKYMYFTVVMAEGKEIDFRCPQDQGWNAEITLQMVQYKNRQAILAVKSTRQKQQHLVQQQPPSQPQPQPQLQPQPQPQPQPQPQPQSQPQPQPQPKPQPQQLHPYPHPHPHPHSHPHSHPHPHPHPHPHQIPHPHPQPHSQPHGHRLLRSTSNSA

SEQ ID NO: 7 - protein, 60 aa, Homo sapiens
MARALYDNVPECAEELAFRKGDILTVIEQNTGGLEGWWLCSLHGRQGIVPGNRVKLLIGP

SEQ ID NO: 8 - protein, 20 aa, Homo sapiens
RALYDNVPECAEELAFRKGD

SEQ ID NO: 9 - protein, 20 aa, Homo sapiens
EALPGEDCAFRKDANRLVEY

SEQ ID NO: 10 - protein, 133 aa, Homo sapiens
ALKEGVLEKRSDGLLQLWKKKCCILTEEGLLLIPPKQLQHQQQQQQQQQQQQQQPGQGPAEPSQPSGPAVASLEPPVKLKELHFSNMKTVDCVERKGKYMYFTVVMAEGKEIDFRCPQDQGWNAEITLQMVQY

SEQ ID NO: 11 - protein, 28 aa, Homo sapiens
KRSDGLLQLWKKKCCILTEEGLLLIPPK

SEQ ID NO: 12 - protein, 19 aa, Homo sapiens
LEPPVKLKELHFSNMKTVD

SEQ ID NO: 13 - protein, 17 aa, Homo sapiens
PQDQGWNAEITLQMVQY

SEQ ID NO: 14 - protein, 20 aa, Homo sapiens
RKGDILTVIEQNTGGLEGWW

SEQ ID NO: 15 - protein, 20 aa, Homo sapiens
SLHGRQGIVPGNRVKLLIGP

SEQ ID NO: 16 - protein, 20 aa, Homo sapiens
RALYDNVPESAEELAFRKGD

SEQ ID NO: 17 - protein, 20 aa, Homo sapiens
AALYDNVPEAAEELAFRKGD

SEQ ID NO: 18 - protein, 15 aa, Homo sapiens
NVPECAEELAFRKGD

SEQ ID NO: 19 - protein, 15 aa, Homo sapiens
RALYDNVPECAEELA

SEQ ID NO: 20 - protein, 15 aa, Homo sapiens
NVPESAEELAFRKGD

SEQ ID NO: 21 - protein, 15 aa, Homo sapiens
RALYDNVPESAEELA

SEQ ID NO: 22 - protein, 15 aa, Homo sapiens
NVPEAAEELAFRKGD

SEQ ID NO: 23 - protein, 15 aa, Homo sapiens
AALYDNVPEAAEELA

SEQ ID NO: 24 - protein, 20 aa, Homo sapiens
RALYANVPACAEELAFRKGD

SEQ ID NO: 25 - protein, 20 aa, Homo sapiens
RALYANVPACAAELAFRKGD

SEQ ID NO: 26 - protein, 20 aa, Homo sapiens
RALYANVPACAAALAFRKGD

SEQ ID NO: 27 - protein, 20 aa, Homo sapiens
RALYANVPASAEELAFRKGD

SEQ ID NO: 28 - protein, 20 aa, Homo sapiens
RALYANVPASAAELAFRKGD

SEQ ID NO: 29 - protein, 20 aa, Homo sapiens
RALYANVPASAAALAFRKGD

SEQ ID NO: 30 - protein, 20 aa, Homo sapiens
RALYANVPAAAEELAFRKGD

SEQ ID NO: 31 - protein, 20 aa, Homo sapiens
RALYANVPAAAAELAFRKGD

SEQ ID NO: 32 - protein, 20 aa, Homo sapiens
RALYANVPAAAAALAFRKGD

SEQ ID NO: 33 - protein, 20 aa, Homo sapiens
AALYDNVPECAEELAFRKGD

SEQ ID NO: 34 - protein, 20 aa, Homo sapiens
RAAYDNVPECAEELAFRKGD

SEQ ID NO: 35 - protein, 20 aa, Homo sapiens
RALADNVPECAEELAFRKGD

SEQ ID NO: 36 - protein, 20 aa, Homo sapiens
RALYANVPECAEELAFRKGD

SEQ ID NO: 37 - protein, 20 aa, Homo sapiens
RALYDAVPECAEELAFRKGD

SEQ ID NO: 38 - protein, 20 aa, Homo sapiens
RALYDNAPECAEELAFRKGD

SEQ ID NO: 39 - protein, 20 aa, Homo sapiens
RALYDNVAECAEELAFRKGD

SEQ ID NO: 40 - protein, 20 aa, Homo sapiens
RALYDNVPACAEELAFRKGD

SEQ ID NO: 41 - protein, 20 aa, Homo sapiens
RALYDNVPEAAEELAFRKGD

SEQ ID NO: 42 - protein, 20 aa, Homo sapiens
RALYDNVPECAAELAFRKGD

SEQ ID NO: 43 - protein, 20 aa, Homo sapiens
RALYDNVPECAEALAFRKGD

SEQ ID NO: 44 - protein, 20 aa, Homo sapiens
RALYDNVPECAEEAAFRKGD

SEQ ID NO: 45 - protein, 20 aa, Homo sapiens
RALYDNVPECAEELAARKGD

SEQ ID NO: 46 - protein, 20 aa, Homo sapiens
RALYDNVPECAEELAFAKGD

SEQ ID NO: 47 - protein, 20 aa, Homo sapiens
RALYDNVPECAEELAFRAGD

SEQ ID NO: 48 - protein, 20 aa, Homo sapiens
RALYDNVPECAEELAFRKAD

SEQ ID NO: 49 - protein, 20 aa, Homo sapiens
RALYDNVPECAEELAFRKGA

SEQ ID NO: 50 - protein, 20 aa, Homo sapiens
AKGDILTVIEQNTGGLEGWW

SEQ ID NO: 51 - protein, 20 aa, Homo sapiens
RAGDILTVIEQNTGGLEGWW

SEQ ID NO: 52 - protein, 20 aa, Homo sapiens
RKADILTVIEQNTGGLEGWW

SEQ ID NO: 53 - protein, 20 aa, Homo sapiens
RKGAILTVIEQNTGGLEGWW

SEQ ID NO: 54 - protein, 20 aa, Homo sapiens [sequence omitted]

SEQ ID NO: 55 - protein, 20 aa, Homo sapiens
RKGDIATVIEQNTGGLEGWW

SEQ ID NO: 56 - protein, 21 aa, Homo sapiens
RKGDILATVIEQNTGGLEGWW

SEQ ID NO: 57 - protein, 20 aa, Homo sapiens
RKGDILTAIEQNTGGLEGWW

SEQ ID NO: 58 - protein, 20 aa, Homo sapiens
RKGDILTVAEQNTGGLEGWW

SEQ ID NO: 59 - protein, 20 aa, Homo sapiens [sequence omitted]

SEQ ID NO: 60 - protein, 20 aa, Homo sapiens [sequence omitted]

SEQ ID NO: 61 - protein, 20 aa, Homo sapiens [sequence omitted]

SEQ ID NO: 62 - protein, 20 aa, Homo sapiens
RKGDILTVIEQNAGGLEGWW

SEQ ID NO: 63 - protein, 20 aa, Homo sapiens
RKGDILTVIEQNTAGLEGWW

SEQ ID NO: 64 - protein, 20 aa, Homo sapiens [sequence omitted]

SEQ ID NO: 65 - protein, 20 aa, Homo sapiens
RKGDILTVIEQNTGGAEGWW

SEQ ID NO: 66 - protein, 20 aa, Homo sapiens
RKGDILTVIEQNTGGLAGWW

SEQ ID NO: 67 - protein, 20 aa, Homo sapiens
RKGDILTVIEQNTGGLEAWW

SEQ ID NO: 68 - protein, 20 aa, Homo sapiens
RKGDILTVIEQNTGGLEGAW

SEQ ID NO: 69 - protein, 20 aa, Homo sapiens
RKGDILTVIEQNTGGLEGWA

SEQ ID NO: 70 - protein, 20 aa, Homo sapiens
ALHGRQGIVPGNRVKLLIGP

SEQ ID NO: 71 - protein, 20 aa, Homo sapiens
SAHGRQGIVPGNRVKLLIGP

SEQ ID NO: 72 - protein, 20 aa, Homo sapiens
SLAGRQGIVPGNRVKLLIGP

SEQ ID NO: 73 - protein, 20 aa, Homo sapiens
SLHARQGIVPGNRVKLLIGP

SEQ ID NO: 74 - protein, 20 aa, Homo sapiens
SLHGAQGIVPGNRVKLLIGP

SEQ ID NO: 75 - protein, 20 aa, Homo sapiens
SLHGRAGIVPGNRVKLLIGP

SEQ ID NO: 76 - protein, 20 aa, Homo sapiens
SLHGRQAIVPGNRVKLLIGP

SEQ ID NO: 77 - protein, 20 aa, Homo sapiens
SLHGRQGAVPGNRVKLLIGP

SEQ ID NO: 78 - protein, 20 aa, Homo sapiens [sequence omitted]

SEQ ID NO: 79 - protein, 20 aa, Homo sapiens
SLHGRQGIVAGNRVKLLIGP

SEQ ID NO: 80 - protein, 20 aa, Homo sapiens [sequence omitted]

SEQ ID NO: 81 - protein, 20 aa, Homo sapiens [sequence omitted]

SEQ ID NO: 82 - protein, 20 aa, Homo sapiens
SLHGRQGIVPGNAVKLLIGP

SEQ ID NO: 83 - protein, 20 aa, Homo sapiens
SLHGRQGIVPGNRAKLLIGP

SEQ ID NO: 84 - protein, 20 aa, Homo sapiens
SLHGRQGIVPGNRVALLIGP

SEQ ID NO: 85 - protein, 20 aa, Homo sapiens
SLHGRQGIVPGNRVKALIGP

SEQ ID NO: 86 - protein, 20 aa, Homo sapiens
SLHGRQGIVPGNRVKLAIGP

SEQ ID NO: 87 - protein, 20 aa, Homo sapiens
SLHGRQGIVPGNRVKLLAGP

SEQ ID NO: 88 - protein, 20 aa, Homo sapiens
SLHGRQGIVPGNRVKLLIAP

SEQ ID NO: 89 - protein, 20 aa, Homo sapiens
SLHGRQGIVPGNRVKLLIGA

SEQ ID NO: 90 - protein, 20 aa, Homo sapiens
AALYDNVPESAEELAFRKGD

SEQ ID NO: 91 - protein, 20 aa, Homo sapiens
RAAYDNVPESAEELAFRKGD

SEQ ID NO: 92 - protein, 20 aa, Homo sapiens
RALADNVPESAEELAFRKGD

SEQ ID NO: 93 - protein, 20 aa, Homo sapiens
RALYANVPESAEELAFRKGD

SEQ ID NO: 94 - protein, 20 aa, Homo sapiens
RALYDAVPESAEELAFRKGD

SEQ ID NO: 95 - protein, 20 aa, Homo sapiens
RALYDNAPESAEELAFRKGD

SEQ ID NO: 96 - protein, 20 aa, Homo sapiens
RALYDNVAESAEELAFRKGD

SEQ ID NO: 97 - protein, 20 aa, Homo sapiens
RALYDNVPASAEELAFRKGD

SEQ ID NO: 98 - protein, 20 aa, Homo sapiens
RALYDNVPESAAELAFRKGD

SEQ ID NO: 99 - protein, 20 aa, Homo sapiens
RALYDNVPESAEALAFRKGD

SEQ ID NO: 100 - protein, 20 aa, Homo sapiens
RALYDNVPESAEEAAFRKGD

SEQ ID NO: 101 - protein, 20 aa, Homo sapiens
RALYDNVPESAEELAARKGD

SEQ ID NO: 102 - protein, 20 aa, Homo sapiens
RALYDNVPESAEELAFAKGD

SEQ ID NO: 103 - protein, 20 aa, Homo sapiens
RALYDNVPESAEELAFRAGD

SEQ ID NO: 104 - protein, 20 aa, Homo sapiens
RALYDNVPESAEELAFRKAD

SEQ ID NO: 105 - protein, 20 aa, Homo sapiens
RALYDNVPESAEELAFRKGA

SEQ ID NO: 106 - protein, 20 aa, Homo sapiens
AALYDNVPEAAEELAFRKGD

SEQ ID NO: 107 - protein, 20 aa, Homo sapiens
RAAYDNVPEAAEELAFRKGD

SEQ ID NO: 108 - protein, 20 aa, Homo sapiens
RALADNVPEAAEELAFRKGD

SEQ ID NO: 109 - protein, 20 aa, Homo sapiens
RALYANVPEAAEELAFRKGD

SEQ ID NO: 110 - protein, 20 aa, Homo sapiens
RALYDAVPEAAEELAFRKGD

SEQ ID NO: 111 - protein, 20 aa, Homo sapiens
RALYDNAPEAAEELAFRKGD

SEQ ID NO: 112 - protein, 20 aa, Homo sapiens
RALYDNVAEAAEELAFRKGD

SEQ ID NO: 113 - protein, 20 aa, Homo sapiens
RALYDNVPAAAEELAFRKGD

SEQ ID NO: 114 - protein, 20 aa, Homo sapiens
RALYDNVPEAAAELAFRKGD

SEQ ID NO: 115 - protein, 20 aa, Homo sapiens
RALYDNVPEAAEALAFRKGD

SEQ ID NO: 116 - protein, 20 aa, Homo sapiens
RALYDNVPEAAEEAAFRKGD

SEQ ID NO: 117 - protein, 20 aa, Homo sapiens
RALYDNVPEAAEELAARKGD

SEQ ID NO: 118 - protein, 20 aa, Homo sapiens
RALYDNVPEAAEELAFAKGD

SEQ ID NO: 119 - protein, 20 aa, Homo sapiens
RALYDNVPEAAEELAFRAGD

SEQ ID NO: 120 - protein, 20 aa, Homo sapiens
RALYDNVPEAAEELAFRKAD

SEQ ID NO: 121 - protein, 20 aa, Homo sapiens
RALYDNVPEAAEELAFRKGA

SEQ ID NO: 122 - protein, 19 aa, Homo sapiens
RALYDNVPECAEELAFRKG

SEQ ID NO: 123 - protein, 18 aa, Homo sapiens
RALYDNVPECAEELAFRK

SEQ ID NO: 124 - protein, 17 aa, Homo sapiens
RALYDNVPECAEELAFR

SEQ ID NO: 125 - protein, 16 aa, Homo sapiens
RALYDNVPECAEELAF

SEQ ID NO: 126 - protein, 14 aa, Homo sapiens
RALYDNVPECAEEL

SEQ ID NO: 127 - protein, 13 aa, Homo sapiens
RALYDNVPECAEE

SEQ ID NO: 128 - protein, 12 aa, Homo sapiens
RALYDNVPECAE

SEQ ID NO: 129 - protein, 11 aa, Homo sapiens
RALYDNVPECA

SEQ ID NO: 130 - protein, 10 aa, Homo sapiens
RALYDNVPEC

SEQ ID NO: 131 - protein, 9 aa, Homo sapiens
RALYDNVPE

SEQ ID NO: 132 - protein, 8 aa, Homo sapiens
RALYDNVP

SEQ ID NO: 133 - protein, 20 aa, Homo sapiens
RALYDNVPECAEELAFRKGD

SEQ ID NO: 134 - protein, 19 aa, Homo sapiens
ALYDNVPECAEELAFRKGD

SEQ ID NO: 135 - protein, 18 aa, Homo sapiens
LYDNVPECAEELAFRKGD

SEQ ID NO: 136 - protein, 17 aa, Homo sapiens
YDNVPECAEELAFRKGD

SEQ ID NO: 137 - protein, 16 aa, Homo sapiens
DNVPECAEELAFRKGD

SEQ ID NO: 138 - protein, 14 aa, Homo sapiens
VPECAEELAFRKGD

SEQ ID NO: 139 - protein, 13 aa, Homo sapiens
PECAEELAFRKGD

SEQ ID NO: 140 - protein, 12 aa, Homo sapiens
ECAEELAFRKGD

SEQ ID NO: 141 - protein, 11 aa, Homo sapiens
CAEELAFRKGD

SEQ ID NO: 142 - protein, 10 aa, Homo sapiens
AEELAFRKGD

SEQ ID NO: 143 - protein, 9 aa, Homo sapiens
EELAFRKGD

SEQ ID NO: 144 - protein, 8 aa, Homo sapiens
ELAFRKGD

SEQ ID NO: 145 - protein, 19 aa, Homo sapiens
RALYDNVPESAEELAFRKG

SEQ ID NO: 146 - protein, 18 aa, Homo sapiens
RALYDNVPESAEELAFRK

SEQ ID NO: 147 - protein, 17 aa, Homo sapiens
RALYDNVPESAEELAFR

SEQ ID NO: 148 - protein, 16 aa, Homo sapiens
RALYDNVPESAEELAF

SEQ ID NO: 149 - protein, 15 aa, Homo sapiens
RALYDNVPESAEELA

SEQ ID NO: 150 - protein, 14 aa, Homo sapiens
RALYDNVPESAEEL

SEQ ID NO: 151 - protein, 13 aa, Homo sapiens
RALYDNVPESAEE

SEQ ID NO: 152 - protein, 12 aa, Homo sapiens
RALYDNVPESAE

SEQ ID NO: 153 - protein, 11 aa, Homo sapiens
RALYDNVPESA

SEQ ID NO: 154 - protein, 10 aa, Homo sapiens
RALYDNVPES

SEQ ID NO: 155 - protein, 20 aa, Homo sapiens
RALYDNVPESAEELAFRKGD

SEQ ID NO: 156 - protein, 19 aa, Homo sapiens
ALYDNVPESAEELAFRKGD

SEQ ID NO: 157 - protein, 18 aa, Homo sapiens
LYDNVPESAEELAFRKGD

SEQ ID NO: 158 - protein, 17 aa, Homo sapiens
YDNVPESAEELAFRKGD

SEQ ID NO: 159 - protein, 16 aa, Homo sapiens
DNVPESAEELAFRKGD

SEQ ID NO: 160 - protein, 15 aa, Homo sapiens
NVPESAEELAFRKGD

SEQ ID NO: 161 - protein, 14 aa, Homo sapiens
VPESAEELAFRKGD

SEQ ID NO: 162 - protein, 13 aa, Homo sapiens
PESAEELAFRKGD

SEQ ID NO: 163 - protein, 12 aa, Homo sapiens
ESAEELAFRKGD

SEQ ID NO: 164 - protein, 11 aa, Homo sapiens
SAEELAFRKGD

SEQ ID NO: 165 - protein, 19 aa, Homo sapiens
RALYDNVPEAAEELAFRKG

SEQ ID NO: 166 - protein, 18 aa, Homo sapiens
RALYDNVPEAAEELAFRK

SEQ ID NO: 167 - protein, 17 aa, Homo sapiens
RALYDNVPEAAEELAFR

SEQ ID NO: 168 - protein, 16 aa, Homo sapiens
RALYDNVPEAAEELAF

SEQ ID NO: 169 - protein, 15 aa, Homo sapiens
RALYDNVPEAAEELA

SEQ ID NO: 170 - protein, 14 aa, Homo sapiens
RALYDNVPEAAEEL

SEQ ID NO: 171 - protein, 13 aa, Homo sapiens
RALYDNVPEAAEE

SEQ ID NO: 172 - protein, 12 aa, Homo sapiens
RALYDNVPEAAE

SEQ ID NO: 173 - protein, 11 aa, Homo sapiens
RALYDNVPEAA

SEQ ID NO: 174 - protein, 10 aa, Homo sapiens
RALYDNVPEA

SEQ ID NO: 175 - protein, 20 aa, Homo sapiens
RALYDNVPEAAEELAFRKGD

SEQ ID NO: 176 - protein, 19 aa, Homo sapiens
ALYDNVPEAAEELAFRKGD

SEQ ID NO: 177 - protein, 18 aa, Homo sapiens
LYDNVPEAAEELAFRKGD

SEQ ID NO: 178 - protein, 17 aa, Homo sapiens
YDNVPEAAEELAFRKGD

SEQ ID NO: 179 - protein, 16 aa, Homo sapiens
DNVPEAAEELAFRKGD

SEQ ID NO: 180 - protein, 15 aa, Homo sapiens
NVPEAAEELAFRKGD

SEQ ID NO: 181 - protein, 14 aa, Homo sapiens
VPEAAEELAFRKGD

SEQ ID NO: 182 - protein, 13 aa, Homo sapiens
PEAAEELAFRKGD

SEQ ID NO: 183 - protein, 12 aa, Homo sapiens
EAAEELAFRKGD

SEQ ID NO: 184 - protein, 11 aa, Homo sapiens
AAEELAFRKGD

SEQ ID NO: 185 - protein, 20 aa, Homo sapiens
MARALYDNVPECAEELAFRK

SEQ ID NO: 186 - protein, 20 aa, Homo sapiens
ARALYDNVPECAEELAFRKG

SEQ ID NO: 187 - protein, 20 aa, Homo sapiens
RALYDNVPECAEELAFRKGD

SEQ ID NO: 188 - protein, 20 aa, Homo sapiens
ALYDNVPECAEELAFRKGDI

SEQ ID NO: 189 - protein, 20 aa, Homo sapiens
LYDNVPECAEELAFRKGDIL

SEQ ID NO: 190 - protein, 20 aa, Homo sapiens
YDNVPECAEELAFRKGDILT

SEQ ID NO: 191 - protein, 20 aa, Homo sapiens
DNVPECAEELAFRKGDILTV

SEQ ID NO: 192 - protein, 20 aa, Homo sapiens
NVPECAEELAFRKGDILTVI

SEQ ID NO: 193 - protein, 20 aa, Homo sapiens
VPECAEELAFRKGDILTVIE

SEQ ID NO: 194 - protein, 20 aa, Homo sapiens
PECAEELAFRKGDILTVIEQ

SEQ ID NO: 195 - protein, 20 aa, Homo sapiens
ECAEELAFRKGDILTVIEQN

SEQ ID NO: 196 - protein, 20 aa, Homo sapiens
CAEELAFRKGDILTVIEQNT

SEQ ID NO: 197 - protein, 20 aa, Homo sapiens
AEELAFRKGDILTVIEQNTG

SEQ ID NO: 198 - protein, 20 aa, Homo sapiens
EELAFRKGDILTVIEQNTGG

SEQ ID NO: 199 - protein, 20 aa, Homo sapiens
ELAFRKGDILTVIEQNTGGL

SEQ ID NO: 200 - protein, 20 aa, Homo sapiens
LAFRKGDILTVIEQNTGGLE

SEQ ID NO: 201 - protein, 20 aa, Homo sapiens
AFRKGDILTVIEQNTGGLEG

SEQ ID NO: 202 - protein, 20 aa, Homo sapiens
FRKGDILTVIEQNTGGLEGW

SEQ ID NO: 203 - protein, 20 aa, Homo sapiens
RKGDILTVIEQNTGGLEGWW

SEQ ID NO: 204 - protein, 20 aa, Homo sapiens
KGDILTVIEQNTGGLEGWWL

SEQ ID NO: 205 - protein, 20 aa, Homo sapiens
GDILTVIEQNTGGLEGWWLC

SEQ ID NO: 206 - protein, 20 aa, Homo sapiens
DILTVIEQNTGGLEGWWLCS

SEQ ID NO: 207 - protein, 20 aa, Homo sapiens
ILTVIEQNTGGLEGWWLCSL

SEQ ID NO: 208 - protein, 20 aa, Homo sapiens
LTVIEQNTGGLEGWWLCSLH

SEQ ID NO: 209 - protein, 20 aa, Homo sapiens
TVIEQNTGGLEGWWLCSLHG

SEQ ID NO: 210 - protein, 20 aa, Homo sapiens
VIEQNTGGLEGWWLCSLHGR

SEQ ID NO: 211 - protein, 20 aa, Homo sapiens
IEQNTGGLEGWWLCSLHGRQ

SEQ ID NO: 212 - protein, 20 aa, Homo sapiens
EQNTGGLEGWWLCSLHGRQG

SEQ ID NO: 213 - protein, 20 aa, Homo sapiens
QNTGGLEGWWLCSLHGRQGI

SEQ ID NO: 214 - protein, 20 aa, Homo sapiens
NTGGLEGWWLCSLHGRQGIV

SEQ ID NO: 215 - protein, 20 aa, Homo sapiens
TGGLEGWWLCSLHGRQGIVP

SEQ ID NO: 216 - protein, 20 aa, Homo sapiens
GGLEGWWLCSLHGRQGIVPG

SEQ ID NO: 217 - protein, 20 aa, Homo sapiens
GLEGWWLCSLHGRQGIVPGN

SEQ ID NO: 218 - protein, 20 aa, Homo sapiens
LEGWWLCSLHGRQGIVPGNR

SEQ ID NO: 219 - protein, 20 aa, Homo sapiens
EGWWLCSLHGRQGIVPGNRV

SEQ ID NO: 220 - protein, 20 aa, Homo sapiens
GWWLCSLHGRQGIVPGNRVK

SEQ ID NO: 221 - protein, 20 aa, Homo sapiens
WWLCSLHGRQGIVPGNRVKL

SEQ ID NO: 222 - protein, 20 aa, Homo sapiens
WLCSLHGRQGIVPGNRVKLL

SEQ ID NO: 223 - protein, 20 aa, Homo sapiens
LCSLHGRQGIVPGNRVKLLI

SEQ ID NO: 224 - protein, 20 aa, Homo sapiens
CSLHGRQGIVPGNRVKLLIG

SEQ ID NO: 225 - protein, 20 aa, Homo sapiens
SLHGRQGIVPGNRVKLLIGP

SEQ ID NO: 226 - protein, 12 aa, Homo sapiens
MARALYDNVPEC

SEQ ID NO: 227 - protein, 12 aa, Homo sapiens
ARALYDNVPECA

SEQ ID NO: 228 - protein, 12 aa, Homo sapiens
RALYDNVPECAE

SEQ ID NO: 229 - protein, 12 aa, Homo sapiens
ALYDNVPECAEE

SEQ ID NO: 230 - protein, 12 aa, Homo sapiens
LYDNVPECAEEL

SEQ ID NO: 231 - protein, 12 aa, Homo sapiens
YDNVPECAEELA

SEQ ID NO: 232 - protein, 12 aa, Homo sapiens
DNVPECAEELAF

SEQ ID NO: 233 - protein, 12 aa, Homo sapiens
NVPECAEELAFR

SEQ ID NO: 234 - protein, 12 aa, Homo sapiens
VPECAEELAFRK

SEQ ID NO: 235 - protein, 12 aa, Homo sapiens
PECAEELAFRKG

SEQ ID NO: 236 - protein, 12 aa, Homo sapiens
ECAEELAFRKGD

SEQ ID NO: 237 - protein, 12 aa, Homo sapiens
CAEELAFRKGDI

SEQ ID NO: 238 - protein, 12 aa, Homo sapiens
AEELAFRKGDIL

SEQ ID NO: 239 - protein, 12 aa, Homo sapiens
EELAFRKGDILT

SEQ ID NO: 240 - protein, 12 aa, Homo sapiens
ELAFRKGDILTV

SEQ ID NO: 241 - protein, 12 aa, Homo sapiens
LAFRKGDILTVI

SEQ ID NO: 242 - protein, 12 aa, Homo sapiens
AFRKGDILTVIE

SEQ ID NO: 243 - protein, 12 aa, Homo sapiens
FRKGDILTVIEQ

SEQ ID NO: 244 - protein, 12 aa, Homo sapiens
RKGDILTVIEQN

SEQ ID NO: 245 - protein, 12 aa, Homo sapiens
KGDILTVIEQNT

SEQ ID NO: 246 - protein, 13 aa, Homo sapiens
GDILTVIEQNTGG

SEQ ID NO: 247 - protein, 13 aa, Homo sapiens
DILTVIEQNTGGL

SEQ ID NO: 248 - protein, 13 aa, Homo sapiens
ILTVIEQNTGGLE

SEQ ID NO: 249 - protein, 13 aa, Homo sapiens
LTVIEQNTGGLEG

SEQ ID NO: 250 - protein, 13 aa, Homo sapiens
TVIEQNTGGLEGW

SEQ ID NO: 251 - protein, 13 aa, Homo sapiens
VIEQNTGGLEGWW

SEQ ID NO: 252 - protein, 13 aa, Homo sapiens
IEQNTGGLEGWWL

SEQ ID NO: 253 - protein, 13 aa, Homo sapiens
EQNTGGLEGWWLC

SEQ ID NO: 254 - protein, 13 aa, Homo sapiens
QNTGGLEGWWLCS

SEQ ID NO: 255 - protein, 13 aa, Homo sapiens
NTGGLEGWWLCSL

SEQ ID NO: 256 - protein, 13 aa, Homo sapiens
TGGLEGWWLCSLH

SEQ ID NO: 257 - protein, 13 aa, Homo sapiens
GGLEGWWLCSLHG

SEQ ID NO: 258 - protein, 13 aa, Homo sapiens
GLEGWWLCSLHGR

SEQ ID NO: 259 - protein, 13 aa, Homo sapiens
LEGWWLCSLHGRQ

SEQ ID NO: 260 - protein, 13 aa, Homo sapiens
EGWWLCSLHGRQG

SEQ ID NO: 261 - protein, 13 aa, Homo sapiens
GWWLCSLHGRQGI

SEQ ID NO: 262 - protein, 13 aa, Homo sapiens
WWLCSLHGRQGIV

SEQ ID NO: 263 - protein, 13 aa, Homo sapiens
WLCSLHGRQGIVP

SEQ ID NO: 264 - protein, 13 aa, Homo sapiens
LCSLHGRQGIVPG

SEQ ID NO: 265 - protein, 13 aa, Homo sapiens
CSLHGRQGIVPGN

SEQ ID NO: 266 - protein, 13 aa, Homo sapiens
SLHGRQGIVPGNR

SEQ ID NO: 267 - protein, 13 aa, Homo sapiens
LHGRQGIVPGNRV

SEQ ID NO: 268 - protein, 13 aa, Homo sapiens
HGRQGIVPGNRVK

SEQ ID NO: 269 - protein, 12 aa, Homo sapiens
RQGIVPGNRVKL

SEQ ID NO: 270 - protein, 12 aa, Homo sapiens
QGIVPGNRVKLL

SEQ ID NO: 271 - protein, 12 aa, Homo sapiens
GIVPGNRVKLLI

SEQ ID NO: 272 - protein, 12 aa, Homo sapiens
IVPGNRVKLLIG

SEQ ID NO: 273 - protein, 12 aa, Homo sapiens
VPGNRVKLLIGP

SEQ ID NO: 274 - protein, 28 aa, Homo sapiens
ARSDGLLQLWKKKCCILTEEGLLLIPPK

SEQ ID NO: 275 - protein, 28 aa, Homo sapiens
KASDGLLQLWKKKCCILTEEGLLLIPPK

SEQ ID NO: 276 - protein, 28 aa, Homo sapiens
KRADGLLQLWKKKCCILTEEGLLLIPPK

SEQ ID NO: 277 - protein, 28 aa, Homo sapiens
KRSAGLLQLWKKKCCILTEEGLLLIPPK

SEQ ID NO: 278 - protein, 28 aa, Homo sapiens
KRSDALLQLWKKKCCILTEEGLLLIPPK

SEQ ID NO: 279 - protein, 28 aa, Homo sapiens
KRSDGALQLWKKKCCILTEEGLLLIPPK

SEQ ID NO: 280 - protein, 28 aa, Homo sapiens
KRSDGLAQLWKKKCCILTEEGLLLIPPK

SEQ ID NO: 281 - protein, 28 aa, Homo sapiens
KRSDGLLALWKKKCCILTEEGLLLIPPK

SEQ ID NO: 282 - protein, 28 aa, Homo sapiens
KRSDGLLQAWKKKCCILTEEGLLLIPPK

SEQ ID NO: 283 - protein, 28 aa, Homo sapiens
KRSDGLLQLAKKKCCILTEEGLLLIPPK

SEQ ID NO: 284 - protein, 28 aa, Homo sapiens
KRSDGLLQLWAKKCCILTEEGLLLIPPK

SEQ ID NO: 285 - protein, 28 aa, Homo sapiens
KRSDGLLQLWKAKCCILTEEGLLLIPPK

SEQ ID NO: 286 - protein, 28 aa, Homo sapiens
KRSDGLLQLWKKACCILTEEGLLLIPPK

SEQ ID NO: 287 - protein, 28 aa, Homo sapiens
KRSDGLLQLWKKKACILTEEGLLLIPPK

SEQ ID NO: 288 - protein, 28 aa, Homo sapiens
KRSDGLLQLWKKKCAILTEEGLLLIPPK

SEQ ID NO: 289 - protein, 28 aa, Homo sapiens
KRSDGLLQLWKKKCCALTEEGLLLIPPK

SEQ ID NO: 290 - protein, 28 aa, Homo sapiens
KRSDGLLQLWKKKCCIATEEGLLLIPPK

SEQ ID NO: 291 - protein, 28 aa, Homo sapiens
KRSDGLLQLWKKKCCILAEEGLLLIPPK

SEQ ID NO: 292 - protein, 28 aa, Homo sapiens
KRSDGLLQLWKKKCCILTAEGLLLIPPK

SEQ ID NO: 293 - protein, 28 aa, Homo sapiens
KRSDGLLQLWKKKCCILTEAGLLLIPPK

SEQ ID NO: 294 - protein, 28 aa, Homo sapiens
KRSDGLLQLWKKKCCILTEEALLLIPPK

SEQ ID NO: 295 - protein, 28 aa, Homo sapiens
KRSDGLLQLWKKKCCILTEEGALLIPPK

SEQ ID NO: 296 - protein, 28 aa, Homo sapiens
KRSDGLLQLWKKKCCILTEEGLALIPPK

SEQ ID NO: 297 - protein, 28 aa, Homo sapiens
KRSDGLLQLWKKKCCILTEEGLLAIPPK

SEQ ID NO: 298 - protein, 28 aa, Homo sapiens
KRSDGLLQLWKKKCCILTEEGLLLAPPK

SEQ ID NO: 299 - protein, 28 aa, Homo sapiens
KRSDGLLQLWKKKCCILTEEGLLLIAPK

SEQ ID NO: 300 - protein, 28 aa, Homo sapiens
KRSDGLLQLWKKKCCILTEEGLLLIPAK

SEQ ID NO: 301 - protein, 28 aa, Homo sapiens
KRSDGLLQLWKKKCCILTEEGLLLIPPA

SEQ ID NO: 302 - protein, 19 aa, Homo sapiens
AEPPVKLKELHFSNMKTVD

SEQ ID NO: 303 - protein, 19 aa, Homo sapiens
LAPPVKLKELHFSNMKTVD

SEQ ID NO: 304 - protein, 19 aa, Homo sapiens
LEAPVKLKELHFSNMKTVD

SEQ ID NO: 305 - protein, 19 aa, Homo sapiens
LEPAVKLKELHFSNMKTVD

SEQ ID NO: 306 - protein, 19 aa, Homo sapiens
LEPPAKLKELHFSNMKTVD

SEQ ID NO: 307 - protein, 19 aa, Homo sapiens
LEPPVALKELHFSNMKTVD

SEQ ID NO: 308 - protein, 19 aa, Homo sapiens
LEPPVKAKELHFSNMKTVD

SEQ ID NO: 309 - protein, 19 aa, Homo sapiens
LEPPVKLAELHFSNMKTVD

SEQ ID NO: 310 - protein, 19 aa, Homo sapiens
LEPPVKLKALHFSNMKTVD

SEQ ID NO: 311 - protein, 19 aa, Homo sapiens
LEPPVKLKEAHFSNMKTVD

SEQ ID NO: 312 - protein, 19 aa, Homo sapiens
LEPPVKLKELAFSNMKTVD

SEQ ID NO: 313 - protein, 19 aa, Homo sapiens
LEPPVKLKELHASNMKTVD

SEQ ID NO: 314 - protein, 19 aa, Homo sapiens
LEPPVKLKELHFANMKTVD

SEQ ID NO: 315 - protein, 19 aa, Homo sapiens
LEPPVKLKELHFSAMKTVD

SEQ ID NO: 316 - protein, 19 aa, Homo sapiens
LEPPVKLKELHFSNAKTVD

SEQ ID NO: 317 - protein, 19 aa, Homo sapiens
LEPPVKLKELHFSNMATVD

SEQ ID NO: 318 - protein, 19 aa, Homo sapiens
LEPPVKLKELHFSNMKAVD

SEQ ID NO: 319 - protein, 19 aa, Homo sapiens
LEPPVKLKELHFSNMKTAD

SEQ ID NO: 320 - protein, 19 aa, Homo sapiens
LEPPVKLKELHFSNMKTVA

SEQ ID NO: 321 - protein, 27 aa, Homo sapiens
KRSDGLLQLWKKKCCILTEEGLLLIPP

SEQ ID NO: 322 - protein, 26 aa, Homo sapiens
KRSDGLLQLWKKKCCILTEEGLLLIP

SEQ ID NO: 323 - protein, 25 aa, Homo sapiens
KRSDGLLQLWKKKCCILTEEGLLLI

SEQ ID NO: 324 - protein, 24 aa, Homo sapiens
KRSDGLLQLWKKKCCILTEEGLLL

SEQ ID NO: 325 - protein, 23 aa, Homo sapiens
KRSDGLLQLWKKKCCILTEEGLL

SEQ ID NO: 326 - protein, 22 aa, Homo sapiens
KRSDGLLQLWKKKCCILTEEGL

SEQ ID NO: 327 - protein, 21 aa, Homo sapiens
KRSDGLLQLWKKKCCILTEEG

SEQ ID NO: 328 - protein, 20 aa, Homo sapiens
KRSDGLLQLWKKKCCILTEE

SEQ ID NO: 329 - protein, 19 aa, Homo sapiens
KRSDGLLQLWKKKCCILTE

SEQ ID NO: 330 - protein, 18 aa, Homo sapiens
KRSDGLLQLWKKKCCILT

SEQ ID NO: 331 - protein, 17 aa, Homo sapiens
KRSDGLLQLWKKKCCIL

SEQ ID NO: 332 - protein, 16 aa, Homo sapiens
KRSDGLLQLWKKKCCI

SEQ ID NO: 333 - protein, 15 aa, Homo sapiens
KRSDGLLQLWKKKCC

SEQ ID NO: 334 - protein, 14 aa, Homo sapiens
KRSDGLLQLWKKKC

SEQ ID NO: 335 - protein, 13 aa, Homo sapiens
KRSDGLLQLWKKK

SEQ ID NO: 336 - protein, 12 aa, Homo sapiens
KRSDGLLQLWKK

SEQ ID NO: 337 - protein, 11 aa, Homo sapiens
KRSDGLLQLWK

SEQ ID NO: 338 - protein, 10 aa, Homo sapiens
KRSDGLLQLW

SEQ ID NO: 339 - protein, 27 aa, Homo sapiens
RSDGLLQLWKKKCCILTEEGLLLIPPK

SEQ ID NO: 340 - protein, 26 aa, Homo sapiens
SDGLLQLWKKKCCILTEEGLLLIPPK

SEQ ID NO: 341 - protein, 25 aa, Homo sapiens
DGLLQLWKKKCCILTEEGLLLIPPK

SEQ ID NO: 342 - protein, 24 aa, Homo sapiens
GLLQLWKKKCCILTEEGLLLIPPK

SEQ ID NO: 343 - protein, 23 aa, Homo sapiens
LLQLWKKKCCILTEEGLLLIPPK

SEQ ID NO: 344 - protein, 22 aa, Homo sapiens
LQLWKKKCCILTEEGLLLIPPK

SEQ ID NO: 345 - protein, 21 aa, Homo sapiens
QLWKKKCCILTEEGLLLIPPK

SEQ ID NO: 346 - protein, 20 aa, Homo sapiens
LWKKKCCILTEEGLLLIPPK

SEQ ID NO: 347 - protein, 19 aa, Homo sapiens
WKKKCCILTEEGLLLIPPK

SEQ ID NO: 348 - protein, 18 aa, Homo sapiens
KKKCCILTEEGLLLIPPK

SEQ ID NO: 349 - protein, 17 aa, Homo sapiens
KKCCILTEEGLLLIPPK

SEQ ID NO: 350 - protein, 16 aa, Homo sapiens
KCCILTEEGLLLIPPK

SEQ ID NO: 351 - protein, 15 aa, Homo sapiens
CCILTEEGLLLIPPK

SEQ ID NO: 352 - protein, 14 aa, Homo sapiens
CILTEEGLLLIPPK

SEQ ID NO: 353 - protein, 13 aa, Homo sapiens
ILTEEGLLLIPPK

SEQ ID NO: 354 - protein, 12 aa, Homo sapiens
LTEEGLLLIPPK

SEQ ID NO: 355 - protein, 11 aa, Homo sapiens
TEEGLLLIPPK

SEQ ID NO: 356 - protein, 10 aa, Homo sapiens
EEGLLLIPPK

SEQ ID NO: 357 - protein, 18 aa, Homo sapiens
EPPVKLKELHFSNMKTVD

SEQ ID NO: 358 - protein, 17 aa, Homo sapiens
PPVKLKELHFSNMKTVD

SEQ ID NO: 359 - protein, 16 aa, Homo sapiens
PVKLKELHFSNMKTVD

SEQ ID NO: 360 - protein, 15 aa, Homo sapiens
VKLKELHFSNMKTVD

SEQ ID NO: 361 - protein, 14 aa, Homo sapiens
KLKELHFSNMKTVD

SEQ ID NO: 362 - protein, 13 aa, Homo sapiens
LKELHFSNMKTVD

SEQ ID NO: 363 - protein, 12 aa, Homo sapiens
KELHFSNMKTVD

SEQ ID NO: 364 - protein, 11 aa, Homo sapiens
ELHFSNMKTVD

SEQ ID NO: 365 - protein, 10 aa, Homo sapiens
LHFSNMKTVD

SEQ ID NO: 366 - protein, 18 aa, Homo sapiens
LEPPVKLKELHFSNMKTV

SEQ ID NO: 367 - protein, 17 aa, Homo sapiens
LEPPVKLKELHFSNMKT

SEQ ID NO: 368 - protein, 16 aa, Homo sapiens
LEPPVKLKELHFSNMK

SEQ ID NO: 369 - protein, 15 aa, Homo sapiens
LEPPVKLKELHFSNM

SEQ ID NO: 370 - protein, 14 aa, Homo sapiens
LEPPVKLKELHFSN

SEQ ID NO: 371 - protein, 13 aa, Homo sapiens
LEPPVKLKELHFS

SEQ ID NO: 372 - protein, 12 aa, Homo sapiens
LEPPVKLKELHF

SEQ ID NO: 373 - protein, 11 aa, Homo sapiens
LEPPVKLKELH

SEQ ID NO: 374 - protein, 10 aa, Homo sapiens
LEPPVKLKEL

SEQ ID NO: 375 - protein, 20 aa, Homo sapiens
ALKEGVLEKRSDGLLQLWKK

SEQ ID NO: 376 - protein, 20 aa, Homo sapiens
LKEGVLEKRSDGLLQLWKKK

SEQ ID NO: 377 - protein, 20 aa, Homo sapiens
KEGVLEKRSDGLLQLWKKKC

SEQ ID NO: 378 - protein, 20 aa, Homo sapiens
EGVLEKRSDGLLQLWKKKCC

SEQ ID NO: 379 - protein, 20 aa, Homo sapiens
GVLEKRSDGLLQLWKKKCCI

SEQ ID NO: 380 - protein, 20 aa, Homo sapiens
VLEKRSDGLLQLWKKKCCIL

SEQ ID NO: 381 - protein, 20 aa, Homo sapiens
LEKRSDGLLQLWKKKCCILT

SEQ ID NO: 382 - protein, 20 aa, Homo sapiens
EKRSDGLLQLWKKKCCILTE

SEQ ID NO: 383 - protein, 20 aa, Homo sapiens
KRSDGLLQLWKKKCCILTEE

SEQ ID NO: 384 - protein, 20 aa, Homo sapiens
RSDGLLQLWKKKCCILTEEG

SEQ ID NO: 385 - protein, 20 aa, Homo sapiens
SDGLLQLWKKKCCILTEEGL

SEQ ID NO: 386 - protein, 20 aa, Homo sapiens
DGLLQLWKKKCCILTEEGLL

SEQ ID NO: 387 - protein, 20 aa, Homo sapiens
GLLQLWKKKCCILTEEGLLL

SEQ ID NO: 388 - protein, 20 aa, Homo sapiens
LLQLWKKKCCILTEEGLLLI

SEQ ID NO: 389 - protein, 20 aa, Homo sapiens
LQLWKKKCCILTEEGLLLIP

SEQ ID NO: 390 - protein, 20 aa, Homo sapiens
QLWKKKCCILTEEGLLLIPP

SEQ ID NO: 391 - protein, 20 aa, Homo sapiens
LWKKKCCILTEEGLLLIPPK

SEQ ID NO: 392 - protein, 20 aa, Homo sapiens
WKKKCCILTEEGLLLIPPKQ

SEQ ID NO: 393 - protein, 20 aa, Homo sapiens
KKKCCILTEEGLLLIPPKQL

SEQ ID NO: 394 - protein, 20 aa, Homo sapiens
KKCCILTEEGLLLIPPKQLQ

SEQ ID NO: 395 - protein, 20 aa, Homo sapiens
KCCILTEEGLLLIPPKQLQH

SEQ ID NO: 396 - protein, 20 aa, Homo sapiens
CCILTEEGLLLIPPKQLQHQ

SEQ ID NO: 397 - protein, 20 aa, Homo sapiens
CILTEEGLLLIPPKQLQHQQ

SEQ ID NO: 398 - protein, 20 aa, Homo sapiens
ILTEEGLLLIPPKQLQHQQQ

SEQ ID NO: 399 - protein, 20 aa, Homo sapiens
LTEEGLLLIPPKQLQHQQQQ

SEQ ID NO: 400 - protein, 20 aa, Homo sapiens
TEEGLLLIPPKQLQHQQQQQ

SEQ ID NO: 401 - protein, 20 aa, Homo sapiens
EEGLLLIPPKQLQHQQQQQQ

SEQ ID NO: 402 - protein, 20 aa, Homo sapiens
EGLLLIPPKQLQHQQQQQQQ

SEQ ID NO: 403 - protein, 20 aa, Homo sapiens
GLLLIPPKQLQHQQQQQQQQ

SEQ ID NO: 404 - protein, 20 aa, Homo sapiens
LLLIPPKQLQHQQQQQQQQQ

SEQ ID NO: 405 - protein, 20 aa, Homo sapiens
LLIPPKQLQHQQQQQQQQQQ

SEQ ID NO: 406 - protein, 20 aa, Homo sapiens
LIPPKQLQHQQQQQQQQQQQ

SEQ ID NO: 407 - protein, 20 aa, Homo sapiens
IPPKQLQHQQQQQQQQQQQQ

SEQ ID NO: 408 - protein, 20 aa, Homo sapiens
PPKQLQHQQQQQQQQQQQQQ

SEQ ID NO: 409 - protein, 20 aa, Homo sapiens
PKQLQHQQQQQQQQQQQQQQ

SEQ ID NO: 410 - protein, 20 aa, Homo sapiens
KQLQHQQQQQQQQQQQQQQP

SEQ ID NO: 411 - protein, 20 aa, Homo sapiens
QLQHQQQQQQQQQQQQQQPG

SEQ ID NO: 412 - protein, 20 aa, Homo sapiens
LQHQQQQQQQQQQQQQQPGQ

SEQ ID NO: 413 - protein, 20 aa, Homo sapiens
QHQQQQQQQQQQQQQQPGQG

SEQ ID NO: 414 - protein, 20 aa, Homo sapiens
HQQQQQQQQQQQQQQPGQGP

SEQ ID NO: 415 - protein, 20 aa, Homo sapiens
QQQQQQQQQQQQQQPGQGPA

SEQ ID NO: 416 - protein, 20 aa, Homo sapiens
QQQQQQQQQQQQQPGQGPAE

SEQ ID NO: 417 - protein, 20 aa, Homo sapiens
QQQQQQQQQQQQPGQGPAEP

SEQ ID NO: 418 - protein, 20 aa, Homo sapiens
QQQQQQQQQQQPGQGPAEPS

SEQ ID NO: 419 - protein, 20 aa, Homo sapiens
QQQQQQQQQQPGQGPAEPSQ

SEQ ID NO: 420 - protein, 20 aa, Homo sapiens
QQQQQQQQQPGQGPAEPSQP

SEQ ID NO: 421 - protein, 20 aa, Homo sapiens
QQQQQQQQPGQGPAEPSQPS

SEQ ID NO: 422 - protein, 20 aa, Homo sapiens
QQQQQQQPGQGPAEPSQPSG

SEQ ID NO: 423 - protein, 20 aa, Homo sapiens
QQQQQQPGQGPAEPSQPSGP

SEQ ID NO: 424 - protein, 20 aa, Homo sapiens
QQQQQPGQGPAEPSQPSGPA

SEQ ID NO: 425 - protein, 20 aa, Homo sapiens
QQQQPGQGPAEPSQPSGPAV

SEQ ID NO: 426 - protein, 20 aa, Homo sapiens
QQQPGQGPAEPSQPSGPAVA

SEQ ID NO: 427 - protein, 20 aa, Homo sapiens
QQPGQGPAEPSQPSGPAVAS

SEQ ID NO: 428 - protein, 20 aa, Homo sapiens
QPGQGPAEPSQPSGPAVASL

SEQ ID NO: 429 - protein, 20 aa, Homo sapiens
PGQGPAEPSQPSGPAVASLE

SEQ ID NO: 430 - protein, 20 aa, Homo sapiens
GQGPAEPSQPSGPAVASLEP

SEQ ID NO: 431 - protein, 20 aa, Homo sapiens
QGPAEPSQPSGPAVASLEPP

SEQ ID NO: 432 - protein, 20 aa, Homo sapiens
GPAEPSQPSGPAVASLEPPV

SEQ ID NO: 433 - protein, 20 aa, Homo sapiens
PAEPSQPSGPAVASLEPPVK

SEQ ID NO: 434 - protein, 20 aa, Homo sapiens
AEPSQPSGPAVASLEPPVKL

SEQ ID NO: 435 - protein, 20 aa, Homo sapiens
EPSQPSGPAVASLEPPVKLK

SEQ ID NO: 436 - protein, 20 aa, Homo sapiens
PSQPSGPAVASLEPPVKLKE

SEQ ID NO: 437 - protein, 20 aa, Homo sapiens
SQPSGPAVASLEPPVKLKEL

SEQ ID NO: 438 - protein, 20 aa, Homo sapiens
QPSGPAVASLEPPVKLKELH

SEQ ID NO: 439 - protein, 20 aa, Homo sapiens
PSGPAVASLEPPVKLKELHF

SEQ ID NO: 440 - protein, 20 aa, Homo sapiens
SGPAVASLEPPVKLKELHFS

SEQ ID NO: 441 - protein, 20 aa, Homo sapiens
GPAVASLEPPVKLKELHFSN

SEQ ID NO: 442 - protein, 20 aa, Homo sapiens
PAVASLEPPVKLKELHFSNM

SEQ ID NO: 443 - protein, 20 aa, Homo sapiens
AVASLEPPVKLKELHFSNMK

SEQ ID NO: 444 - protein, 20 aa, Homo sapiens
VASLEPPVKLKELHFSNMKT

SEQ ID NO: 445 - protein, 20 aa, Homo sapiens
ASLEPPVKLKELHFSNMKTV

SEQ ID NO: 446 - protein, 20 aa, Homo sapiens
SLEPPVKLKELHFSNMKTVD

SEQ ID NO: 447 - protein, 20 aa, Homo sapiens
LEPPVKLKELHFSNMKTVDC

SEQ ID NO: 448 - protein, 20 aa, Homo sapiens
EPPVKLKELHFSNMKTVDCV

SEQ ID NO: 449 - protein, 20 aa, Homo sapiens
PPVKLKELHFSNMKTVDCVE

SEQ ID NO: 450 - protein, 20 aa, Homo sapiens
PVKLKELHFSNMKTVDCVER

SEQ ID NO: 451 - protein, 20 aa, Homo sapiens
VKLKELHFSNMKTVDCVERK

SEQ ID NO: 452 - protein, 20 aa, Homo sapiens
KLKELHFSNMKTVDCVERKG

SEQ ID NO: 453 - protein, 20 aa, Homo sapiens
LKELHFSNMKTVDCVERKGK

SEQ ID NO: 454 - protein, 20 aa, Homo sapiens
KELHFSNMKTVDCVERKGKY

SEQ ID NO: 455 - protein, 20 aa, Homo sapiens
ELHFSNMKTVDCVERKGKYM

SEQ ID NO: 456 - protein, 20 aa, Homo sapiens
LHFSNMKTVDCVERKGKYMY

SEQ ID NO: 457 - protein, 20 aa, Homo sapiens
HFSNMKTVDCVERKGKYMYF

SEQ ID NO: 458 - protein, 20 aa, Homo sapiens
FSNMKTVDCVERKGKYMYFT

SEQ ID NO: 459 - protein, 20 aa, Homo sapiens
SNMKTVDCVERKGKYMYFTV

SEQ ID NO: 460 - protein, 20 aa, Homo sapiens
NMKTVDCVERKGKYMYFTVV

SEQ ID NO: 461 - protein, 20 aa, Homo sapiens
MKTVDCVERKGKYMYFTVVM

SEQ ID NO: 462 - protein, 20 aa, Homo sapiens
KTVDCVERKGKYMYFTVVMA

SEQ ID NO: 463 - protein, 20 aa, Homo sapiens
TVDCVERKGKYMYFTVVMAE

SEQ ID NO: 464 - protein, 20 aa, Homo sapiens
VDCVERKGKYMYFTVVMAEG

SEQ ID NO: 465 - protein, 20 aa, Homo sapiens
DCVERKGKYMYFTVVMAEGK

SEQ ID NO: 466 - protein, 20 aa, Homo sapiens
CVERKGKYMYFTVVMAEGKE

SEQ ID NO: 467 - protein, 20 aa, Homo sapiens
VERKGKYMYFTVVMAEGKEI

SEQ ID NO: 468 - protein, 20 aa, Homo sapiens
ERKGKYMYFTVVMAEGKEID

SEQ ID NO: 469 - protein, 20 aa, Homo sapiens
RKGKYMYFTVVMAEGKEIDF

SEQ ID NO: 470 - protein, 20 aa, Homo sapiens
KGKYMYFTVVMAEGKEIDFR

SEQ ID NO: 471 - protein, 20 aa, Homo sapiens
GKYMYFTVVMAEGKEIDFRC

SEQ ID NO: 472 - protein, 20 aa, Homo sapiens
KYMYFTVVMAEGKEIDFRCP

SEQ ID NO: 473 - protein, 20 aa, Homo sapiens
YMYFTVVMAEGKEIDFRCPQ

SEQ ID NO: 474 - protein, 20 aa, Homo sapiens
MYFTVVMAEGKEIDFRCPQD

SEQ ID NO: 475 - protein, 20 aa, Homo sapiens
YFTVVMAEGKEIDFRCPQDQ

SEQ ID NO: 476 - protein, 20 aa, Homo sapiens
FTVVMAEGKEIDFRCPQDQG

SEQ ID NO: 477 - protein, 20 aa, Homo sapiens
TVVMAEGKEIDFRCPQDQGW

SEQ ID NO: 478 - protein, 20 aa, Homo sapiens
VVMAEGKEIDFRCPQDQGWN

SEQ ID NO: 479 - protein, 20 aa, Homo sapiens
VMAEGKEIDFRCPQDQGWNA

SEQ ID NO: 480 - protein, 20 aa, Homo sapiens
MAEGKEIDFRCPQDQGWNAE

SEQ ID NO: 481 - protein, 20 aa, Homo sapiens
AEGKEIDFRCPQDQGWNAEI

SEQ ID NO: 482 - protein, 20 aa, Homo sapiens
EGKEIDFRCPQDQGWNAEIT

SEQ ID NO: 483 - protein, 20 aa, Homo sapiens
GKEIDFRCPQDQGWNAEITL

SEQ ID NO: 484 - protein, 20 aa, Homo sapiens
KEIDFRCPQDQGWNAEITLQ

SEQ ID NO: 485 - protein, 20 aa, Homo sapiens
EIDFRCPQDQGWNAEITLQM

SEQ ID NO: 486 - protein, 20 aa, Homo sapiens
IDFRCPQDQGWNAEITLQMV

SEQ ID NO: 487 - protein, 20 aa, Homo sapiens
DFRCPQDQGWNAEITLQMVQ

SEQ ID NO: 488 - protein, 20 aa, Homo sapiens
FRCPQDQGWNAEITLQMVQY

(19) United States
(12) Patent Application Publication
(10) Pub. No.: US 2010/0233199 A1
(43) Pub. Date: Sep. 16, 2010

(54) MODULATORS OF HYPERSENSITIVITY REACTIONS

(76) Inventors: Diego Silva, Kingston (AU); David Linares Bandin, Lyons (AU); Peter J. Milburn, O'Connor (AU)

Correspondence Address: BUCHANAN INGERSOLL & ROONEY PC, P.O. BOX 1404, ALEXANDRIA, VA, 22313-1404, US

(21) Appl. No.: 12/674,739

(22) PCT Filed: Aug. 25, 2008
(86) PCT No.: PCT/AU08/01250

§ 371 Date: May 13, 2010

(30) Foreign Application Priority Data
Aug. 24, 2007 (AU) 2007904591

(51) Int. Cl. A61K 39/00; C07K 14/00; A61P 37/08

(52) U.S. Cl. 424/185.1; 530/350

(57) ABSTRACT

The present invention relates to a peptide for modulating an immune response, said peptide comprising an amino acid sequence corresponding to at least a portion of one of a Src homology 3 (SH3) domain, a Src homology 2 (SH2) domain, or a pleckstrin homology (PH) domain.

### TECHNICAL FIELD

The invention relates to modulators of mast cell activation and in particular their use in the treatment and prevention of hypersensitivity diseases and disorders. More specifically, the invention relates to proteins and peptides capable of inhibiting and/or preventing mast cell activation.

### BACKGROUND

The prevalence of hypersensitivity-associated disorders has dramatically increased in the industrialized world over the last decades. In particular, the most common type of hypersensitivity-associated disorder, IgE-mediated hypersensitivity, already effects more than 25% of the population in developing countries.

Hypersensitivity reactions are the result of immune responses acting inappropriately and can be provoked by many antigens. They are produced by a combination of inflammatory mediators released by several cell types. One form of hypersensitivity occurs when an IgE response is directed against innocuous environmental antigens, such as pollen or dust-mites. The resulting release of pharmacological mediators by IgE-sensitized mast cells produces an acute inflammatory reaction with symptoms such as asthma or rhinitis. Airway inflammation is central to the pathogenesis of asthma and involves the recruitment and activation of mast cells, eosinophils, neutrophils, and lymphocytes into lung tissue and bronchoalveolar space.

Mast cells are considered to be one of the key effectors in hypersensitivity reactions. These cells are distributed throughout the body, in close proximity to blood vessels, nerves, mucosal surfaces and skin. At those sites, they are well positioned to detect allergens and to initiate the earliest phases of the allergic response by their ability to release several inflammatory mediators. Mast cells are amongst the only cells in the body to express a receptor specific for IgE, which is the immunoglobulin responsible for developing allergic responses and asthma.

Mast cells can be activated by various stimuli, however, the most specific is via the interaction of the antigen with IgE bound to its high affinity receptor (FcεRI) on the cell surface. Antigen detection by FcεRIα-bound IgE leads to receptor cross-linking and subsequent phosphorylation of the tyrosine residues in the ITAM motif of the β and γ subunits of the same receptor. Consequently, aggregation of the FcεRI receptor induces the activation of a complex intracellular pathway involving numerous protein interactions which eventually lead to the release of preformed granules and the secretion of eicosanoids, cytokines and chemokines. Mast cell activation leads to a series of biochemical events causing the systemic and/or local effects which are typically observed in allergic and anaphylactic reactions.

Current protocols used for the treatment of hypersensitivity-associated disorders mainly comprise a combination of glucocorticoids and other symptom relieving medications. Rather than directly targeting mast cells, these treatments are aimed at interfering with the effects of downstream inflammatory mediators. Anti-inflammatory drugs such as corticoids influence a variety of cellular functions in a non-selective manner. Therefore, complications arising from long term steroid therapy have imposed limitations on its clinical use.

There is a need for new therapeutic approaches for the treatment of hypersensitivity-associated diseases and disorders. In particular, a need exists for new therapeutic approaches targeting mast cells in order to modulate the release of inflammatory mediators associated with allergic responses.

### SUMMARY OF THE INVENTION

Described herein are genes previously unknown to be involved in mast cell activation and the onset of hypersensitivity reactions. The proteins encoded by those genes comprise at least one of a pleckstrin homology (PH), an Src homology 2 (SH2), or an Src homology 3 (SH3) domain. These domains are believed to modulate mast cell activation via specific interactions with receptors present on the mast cell surface. Certain aspects and embodiments of the invention thus relate to the modulation of mast cell activation by administration of SH2/SH3/PH domain containing proteins. Also described herein are peptides corresponding to SH3 and/or PH domain sequences capable of modulating mast cell activation. The peptides described herein provide a means of treating and preventing hypersensitivity diseases and disorders.

In a first aspect, the invention provides a peptide for modulating mast cell activation, said peptide comprising an amino acid sequence corresponding to at least a portion of:

(i) an Src homology 3 (SH3) domain, or

(ii) a pleckstrin homology (PH) domain.

In one embodiment of the first aspect, the peptide comprises an amino acid set forth in SEQ ID NO: 7.

In another embodiment of the first aspect, the peptide comprises an amino acid set forth in SEQ ID NO: 8, or any one of SEQ ID NOs: 14-273.

In another embodiment of the first aspect, the peptide comprises an amino acid set forth in SEQ ID NO: 11 or SEQ ID NO: 12.

In one embodiment of the first aspect, the peptide comprises an amino acid set forth in SEQ ID NO: 10 or any one of SEQ ID NOs: 274-488.

In another embodiment of the first aspect, said modulating inhibits mast cell activation.

In a second aspect, the invention provides a method of inhibiting or preventing mast cell activation in a subject, the method comprising administering to the subject a therapeutically effective amount of a protein comprising at least one of:

(i) an Src homology 2 (SH2) domain,

(ii) an Src homology 3 (SH3) domain,

(iii) a pleckstrin homology (PH) domain,

(iv) the peptide of any one of claims 1-5.

In one embodiment of the second aspect, the mast cell activation is IgE-mediated.

In another embodiment of the second aspect, the mast cell activation is non IgE-mediated.

In a third aspect, the invention provides a method for treating or preventing a hypersensitivity disease or disorder in a subject, the method comprising administering to the subject a therapeutically effective amount of a protein comprising at least one of:

(i) an Src homology 2 (SH2) domain,

(ii) an Src homology 3 (SH3) domain,

(iii) a pleckstrin homology (PH) domain,

(iv) the peptide of any one of claims 1-5.

In one embodiment of the third aspect, the hypersensitivity disease or disorder comprises mast cell activation.

In another embodiment of the third aspect, the hypersensitivity disease or disorder comprises an inflammatory reaction.

In another embodiment of the third aspect, the hypersensitivity disease or disorder may be anaphylaxis, drug reactions, skin allergy, eczema, allergic rhinitis, urticaria, atopic dermatitis, allergic contact allergy, food allergy, allergic conjunctivitis, insect venom allergy and respiratory diseases and disorders. The respiratory disease or disorder may be asthma, allergic asthma, intrinsic asthma, occupational asthma, acute respiratory distress syndrome (ARDS) and chronic obstructive pulmonary disease (COPD).

In a fourth aspect, the invention provides a method of modulating an immune response in a subject, the method comprising administering to the subject a therapeutically effective amount of a protein comprising at least one of:

(i) an Src homology 2 (SH2) domain,

(ii) an Src homology 3 (SH3) domain,

(iii) a pleckstrin homology (PH) domain,

(iv) the peptide of any one of claims 1-5.

In one embodiment of the fourth aspect, the protein is NEDD9.

In one embodiment of the fourth aspect, the protein is PHLDA1.

In a fifth aspect, the invention provides a method of inhibiting or preventing mast cell activation in a subject, the method comprising administering to the subject a therapeutically effective amount of a peptide, the peptide comprising an amino acid sequence corresponding to at least a portion of:

(i) an Src homology 3 (SH3) domain, or

(ii) a pleckstrin homology (PH) domain.

In a sixth aspect, the invention provides a method of treating or preventing a hypersensitivity disease or disorder in a subject, the method comprising administering to the subject a therapeutically effective amount of a peptide, the peptide comprising an amino acid sequence corresponding to at least a portion of:

(i) an Src homology 3 (SH3) domain, or

(ii) a pleckstrin homology (PH) domain.

In one embodiment of the sixth aspect, the hypersensitivity disease or disorder comprises mast cell activation.

In another embodiment of the sixth aspect, the hypersensitivity disease or disorder comprises an inflammatory reaction.

In an additional embodiment of the sixth aspect, the hypersensitivity disease or disorder is selected from the group comprising anaphylaxis, drug reactions, skin allergy, eczema, allergic rhinitis, urticaria, atopic dermatitis, allergic contact allergy, food allergy, allergic conjunctivitis, insect venom allergy and respiratory diseases and disorders. The respiratory disease or disorder may be asthma, allergic asthma, intrinsic asthma, occupational asthma, acute respiratory distress syndrome (ARDS) and chronic obstructive pulmonary disease (COPD).

In a seventh aspect, the invention provides a method of modulating an immune response in a subject, the method comprising administering to the subject a therapeutically effective amount of a peptide, the peptide comprising an amino acid sequence corresponding to at least a portion of

(i) an Src homology 3 (SH3) domain, or

(ii) a pleckstrin homology (PH) domain.

In one embodiment of the fifth, sixth and seventh aspects, the peptide comprises the amino acid sequence set forth in SEQ ID NO: 7 or a variant.

In an additional embodiment of the fifth, sixth and seventh aspects, the peptide comprises the amino acid sequence set forth in SEQ ID NO: 8 or a variant.

In another embodiment of the fifth, sixth and seventh aspects, the peptide comprises the amino acid sequence set forth in SEQ ID NO: 11.

In yet another embodiment of the fifth, sixth and seventh aspects, the peptide comprises the amino acid sequence set forth in SEQ ID NO: 12 or a variant.

In an eighth aspect, the invention provides a method of inhibiting mast cell activation, the method comprising inhibiting the binding of either of both of:

(i) a NEDD9 protein Src homology 3 (SH3) domain

(ii) a PHLDA1 protein pleckstrin homology (PH) domain to a mast cell receptor.

In a ninth aspect, the invention provides a method for treating or preventing a hypersensitivity disease or disorder in a subject, the method comprising inhibiting the binding of either of both of

(i) a NEDD9 protein Src homology 3 (SH3) domain

(ii) a PHLDA1 protein pleckstrin homology (PH) domain to a mast cell receptor.

In one embodiment of the eighth and ninth aspects, the inhibiting comprises administration of a peptide comprising an amino acid sequence corresponding to at least a portion of the NEDD9 protein Src homology 3 (SH3) domain or the PHLDA1 protein pleckstrin homology (PH) domain. The peptide may comprise an amino acid sequence set forth in SEQ ID NO: 7 or SEQ ID NO:8. The peptide may comprise an amino acid selected form the group consisting of SEQ ID NO: 10, SEQ ID NO: 11 and SEQ ID NO:12.

In a tenth aspect, the invention provides the use of a protein comprising at least one of:

(i) an Src homology 2 (SH2) domain,

(ii) an Src homology 3 (SH3) domain,

(iii) a pleckstrin homology (PH) domain,

(iv) the peptide of any one of claims 1-5.

for the manufacture of a medicament for the treatment of a hypersensitivity disease or disorder.

In one embodiment of the tenth aspect, the protein is NEDD9.

In another embodiment of the tenth aspect, the protein is PHLDA1.

In an eleventh aspect, the invention provides the use of a peptide comprising an amino acid sequence corresponding to at least a portion of:

(i) an Src homology 3 (SH3) domain, or

(ii) a pleckstrin homology (PH) domain,

for the manufacture of a medicament for the treatment of a hypersensitivity disease or disorder.

In one embodiment of the eleventh aspect, the peptide comprises an amino acid sequence selected from the group consisting of SEQ ID NO: 7, SEQ ID NO: 8, SEQ ID NO: 11 and SEQ ID NO: 12.

In one embodiment of the tenth and eleventh aspects, the hypersensitivity disease or disorder is selected from the group consisting of anaphylaxis, drug reactions, skin allergy, eczema, allergic rhinitis, urticaria, atopic dermatitis, allergic contact allergy, food allergy, allergic conjunctivitis, insect venom allergy and respiratory diseases and disorders. The respiratory disease or disorder may be asthma, allergic asthma, intrinsic asthma, occupational asthma, acute respiratory distress syndrome (ARDS) and chronic obstructive pulmonary disease (COPD).

In an twelfth aspect, the invention provides a protein comprising at least one of:

(i) an Src homology 2 (SH2) domain,

(ii) an Src homology 3 (SH3) domain,

(iii) a pleckstrin homology (PH) domain,

(iv) the peptide of any one of claims 1-5.

for the treatment of a hypersensitivity disease or disorder.

In an thirteenth aspect, the invention provides a peptide comprising an amino acid sequence corresponding to at least a portion of:

(i) an Src homology 3 (SH3) domain, or

(ii) a pleckstrin homology (PH) domain,

for the treatment of a hypersensitivity disease or disorder.

In an fourteenth aspect, the invention provides a method of identifying an agent that modulates the activity of a protein comprising at least one of an Src homology 2 (SH2) domain, an Src homology 3 (SH3) domain, or a pleckstrin homology (PH) domain, the method comprising:

(a) contacting a candidate agent with the protein under conditions suitable to permit interaction of the candidate agent with the protein; and

(b) assaying the activity of the protein.

In an fifteenth aspect, the invention provides a method for screening a plurality of candidate agents to identify an agent that modulates the activity of a protein comprising at least one of an Src homology 2 (SH2) domain, an Src homology 3 (SH3) domain, or a pleckstrin homology (PH) domain, the method comprising:

(a) contacting a plurality of candidate agents with the protein under conditions suitable to permit interaction of the candidate agent with the protein; and

(b) assaying the activity of the protein.

In one embodiment of the fourteenth and fifteenth aspects, the protein is NEDD9 or PHLDA1

In another embodiment of the fourteenth and fifteenth aspects, assaying the activity of the protein comprises measuring the level of mast cell activation.

In an additional embodiment of the fourteenth and fifteenth aspects, the candidate agent is an agonist of the protein.

In a further embodiment of the fourteenth and fifteenth aspects, the candidate agent is an agonist of the protein.

### Abbreviations

BMMC Bone marrow derived mast cells
DEAE dextran diethylaminoethyl dextran
DNA deoxyribonucleic acid
DNP-IgE anti-dinitrophenol immunoglobulin E
dsRNA double-stranded RNA
Fc constant fragment
FcεRI Fc epsilon receptor I, high affinity IgE receptor
FcεRIα α-subunit of high-affinity IgE receptor
FcγR Fc gamma receptor
Fyn tyrosine-protein kinase Fyn
IgE immunoglobulin E
IgG immunoglobulin G
IL-10 interleukin 10
ITAM immunoreceptor tyrosine-based activation motif
kg kilogram
LAMP-1 lysosome-associated membrane protein-1
LAMP-2 lysosome-associated membrane protein-1
Lck lymphocyte-specific protein tyrosine kinase
Lyn tyrosine-protein kinase Lyn
m meter
mg milligram
mRNA messenger ribonucleic acid
NEDD9 neural precursor cell-expressed developmentally downregulated gene 9
PH pleckstrin homology
PHLDA1 pleckstrin homology-like domain family A member 1
PHRIP proline and histidine rich protein
RBL-2H3 cells rat basophilic leukaemia 2H3 cells
RNA ribonucleic acid
SDS-PAGE sodium dodecyl sulphate polyacrylamide gel electrophoresis
SH2 Src homology 2 domain
SH3 Src homology 3 domain
siRNA small inhibitory RNA
TDAG51 T-cell death associated gene 51

### DEFINITIONS

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

As used herein, an “agent” includes within its scope any natural or manufactured element or compound. Accordingly, the term includes, but is not limited to any: chemical elements and chemical compounds, nucleic acids, amino acids, polypeptides, proteins, antibodies and fragments of antibodies, and other substances that may be appropriate in the context of the invention.

As used herein the terms “modulating”, “modulates” and variations thereof refer to increasing or decreasing the level of activity, production, secretion or functioning of a molecule in the presence of a particular modulatory molecule or agent of the invention compared to the level of activity, production, secretion or other functioning thereof in the absence of the modulatory molecule or agent. These terms do not imply quantification of the increase or decrease. The modulation may be of any magnitude sufficient to produce the desired result and may be direct or indirect.

As used herein, the term “immunomodulator” refers to a molecular mediator secreted by one or more cell types and which plays a role in the activation, maintenance, maturation, inhibition, suppression or augmentation of an immune response.

As used herein, the term “SH2/SH3/PH domain containing protein” refers to a protein containing one or more of:

(a) at least one Src homology 2 (SH2) domain,

(b) at least one Src homology 3 (SH3) domain,

(c) at least one pleckstrin homology (PH) domain.

Hence, an SH2/SH3/PH domain containing protein encompasses a protein with one or more SH2 domains only, with one or more SH3 domains only, with one or more PH domains only, with one or more SH2 domains and one or more SH3 domains, with one or more SH3 domains and one or more PH domains, or with one or more SH2 and one or more SH3 domains and one or more PH domains.

As used herein, the term “mast cell activation pathway” includes within its scope all components and steps in the biological processes occurring within or on the surface of a mast cell leading to its activation. Generally, the activation of a mast cell may be initiated by external stimuli, leading to an intracellular signaling cascade involving a series of cellular proteins, for example, phosphatases, kinases and adaptor/regulatory factors, causing activation of the mast cell.

As used herein, the term “administering” and variations of that term including “administer” and “administration”, includes contacting, applying, delivering or providing a compound or composition of the invention to an organism by any appropriate means.

As used herein, the terms “antibody” and “antibodies” include IgG (including IgG1, IgG2, IgG3, and IgG4), IgA (including IgA1 and IgA2), IgD, IgE, or IgM, and IgY, whole antibodies, including single-chain whole antibodies, and antigen-binding fragments thereof. Antigen-binding antibody fragments include, but are not limited to, Fab, Fab′ and F(ab′)2, Fd, single-chain Fvs (scFv), single-chain antibodies, disulfide-linked Fvs (sdFv) and fragments comprising either a VL or VH domain. The antibodies may be from any animal origin. Antigen-binding antibody fragments, including single-chain antibodies, may comprise the variable region(s) alone or in combination with the entire or partial of the following: hinge region, CH1, CH2, and CH3 domains. Also included are any combinations of variable region(s) and hinge region, CH1, CH2, and CH3 domains. Antibodies may be monoclonal, polyclonal, chimeric, multispecific, humanized, and human monoclonal and polyclonal antibodies which specifically bind the biological molecule.

As used herein, the term “nucleic acid” refers to a deoxyribonucleotide or ribonucleotide polymer in either single-or double-stranded form, and unless otherwise limited, encompasses known analogues of natural nucleotides that hybridize to nucleic acids in a manner similar to naturally occurring nucleotides.

As used herein, the term “polypeptide” or “peptide” means a polymer made up of amino acids linked together by peptide bonds. The terms “polypeptide” and “peptide” are used interchangeably herein, although for the purposes of the present invention a “polypeptide” may constitute a portion of a full length protein.

As used herein, the term “polynucleotide” refers to a single- or double-stranded polymer of deoxyribonucleotide, ribonucleotide bases or known analogues or natural nucleotides, or mixtures thereof.

As used herein, the term “mutation” as used throughout the specification is intended to encompass any and all types of functional and/or non-functional nucleic acid changes, including mutations and polymorphisms in the target nucleic acid molecule when compared to a wildtype variant of the same nucleic acid region or allele or the more common nucleic acid molecule present on the sample. Such changes, include, but are not limited to deletions, insertions, translocations, inversions, and base substitutions of one or more nucleotides.

As used herein, the term “polymorphism” refers to a variation in the sequence of a gene in the genome amongst a population, such as allelic variations and other variations that arise or are observed. Genetic polymorphisms refer to the variant forms of gene sequences that can arise as a result of nucleotide base pair differences, alternative mRNA splicing or post-translational modifications, including, for example, glycosylation. Thus, a polymorphism refers to the occurrence of two or more genetically determined alternative sequences or alleles in a population. These differences can occur in coding and non-coding portions of the genome, and can be manifested or detected as differences in nucleic acid sequences, gene expression, including, for example transcription, processing, translation, transport, protein processing, trafficking, DNA synthesis, expressed proteins, other gene products or products of biochemical pathways or in post-translational modifications and any other differences manifested.

As used herein the term “treatment”, refers to any and all uses which remedy a disease state or symptoms, or otherwise prevent, hinder, retard, or reverse the progression of disease or other undesirable symptoms in any way whatsoever.

As used herein, the term “subject” includes humans and individuals of any mammalian species of social, economic or research importance including but not limited to members of the genus ovine, bovine, equine, porcine, feline, canine, primates, and rodents. In one embodiment, the mammal is a human.

As used herein the terms “effective amount” and “therapeutically effective amount” include within their meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention before the priority date of this application. For the purposes of description all documents referred to herein are incorporated by reference.

### BRIEF DESCRIPTION OF THE DRAWINGS

FIG. 1A shows a graph illustrating a timecourse of the relative gene expression of NEDD9 in human mast cells in response to IgE stimulation.

FIG. 1B shows a graph illustrating a timecourse of the relative gene expression of PHLDA1 in human mast cells in response to IgE stimulation.

FIG. 1C shows a graph illustrating a timecourse of the relative gene expression of NEDD9 in mouse mast cells in response to IgE stimulation.

FIG. 1D shows a graph illustrating a timecourse of the relative gene expression of PHLDA1 in mouse mast cells in response to IgE stimulation.

FIG. 2A shows a graph illustrating relative NEDD9 mRNA expression in RBL-2H3 cells in response to IgE stimulation. Results are expressed as relative fold change over the values for untreated samples.

FIG. 2B shows a graph illustrating relative NEDD9 mRNA expression in RBL-2H3 cells in response to ionomycin stimulation. Results are expressed as relative fold change over the values for untreated samples.

FIG. 2C shows a graph illustrating relative PHLDA1 mRNA expression in RBL-2H3 cells in response to IgE stimulation. Results are expressed as relative fold change over the values for untreated samples.

FIG. 2D shows a graph illustrating relative PHLDA1 mRNA expression in RBL-2H3 cells in response to ionomycin stimulation. Results are expressed as relative fold change over the values for untreated samples.

FIG. 3 shows a graph illustrating the percentage of mast cell degranulation in RBL-2H3 cells incubated with IgE for 30 minutes in the presence of 3 different siRNAs targeting NEDD9 gene expression. An additional siRNA not targeted to any gene was used as a control.

FIG. 4A shows a graph illustrating the degree of mast cell degranulation as measured by Evan's blue recovery in Lewis rats following administration of a combination of siRNA for NEDD9 with DNP-IgE, compared to a negative siRNA/DNP-IgE control.

FIG. 4B shows a graph illustrating the degree of mast cell degranulation as measured by Evan's blue recovery in Lewis rats following administration of a combination of siRNA for PHLDA1 with DNP-IgE, compared to a negative siRNA/DNP-IgE control.

FIG. 5 shows an amino acid sequence alignment of the NEDD9 SH3 domain in Homo sapiens (HS), Mus musculus (MM), Rattus norvegicus (RN), Pan troglodytes (PT), Bos taurus (BT), Canis familiaris (CF) and gallus gallus (GG). Highlighted residues indicate the putative active site (amino acids 9-28 in HS, MM, RN, PT and BT; amino acid residues 41-60 in CF; amino acid residues 41-59 in GG).

FIG. 6 is a histogram showing biotinylated MDD1 uptake in live RBL-2H3 cells, detected by FACS following intracellular staining with strepatvidin-APC. Biotinylated MDD1 was administered to cells at a concentration of 0.001 mM MDD1 (2), 0.01 mM MDD1 (3) or 0.1 mM MDD1 (4). Control cells were administered PBS alone (1).

FIG. 7 is a graph showing the effect of the MDD1 peptide on RBL-2H3 cell degranulation following IgE receptor crosslinking. (1) IgE coating only (2) IgE coating+IgE receptor crosslinking (3) MDD1 peptide (0.01 mM) pre-treatment+IgE coating+IgE receptor crosslinking (4) SCR peptide 0.01 mM pre-treatment+IgE coating+IgE receptor crosslinking.

FIG. 8 provides graphs showing the effects of MDD1 peptides on lung function in mice sensitized with ovalbumin antigen (OVA). Mice intranasally challenged with OVA to induce asthma were co-administered MDD1 peptide (A) intravenously at 8 mg/kg or (B) intranasally by nebulisation at 1 mg/ml. Lung resistance was measured by plethysmography. RL: Lung resistance.

FIG. 9 provides graphs showing the level of the inflammatory response in OVA-sensitized mice upon induction of asthma. Asthma was induced in OVA-sensitized mice pre-treated with either saline, SCR control peptide or MDD1 peptide by challenge with β-methacholine. (A) % Eosinophils in blood (B) Total cells in croncheolar lavage (C) TNF expression at inflammatory foci and (D) levels of antigen-specific IgE in serum.

FIG. 10 is a graph illustrating that topical administration of MDD1 peptide stabilises skin mast cells derived from ear biopsies of IgE sensitized mice following IgE receptor crosslinking. Left (L) ears from mice were treated with DMSO alone. Right (R) ears were treated with a mixture of either MDD1 peptide/DMSO or dexamethasone/DMSO.

FIG. 11 is a graph showing the effect of MPX741 and MPX742 peptides on RBL-2H3 cell degranulation following IgE receptor crosslinking. (1) IgE coating only (2) IgE coating+IgE receptor crosslinking (3) MPX741 peptide (0.01M) pre-treatment+IgE coating+IgE receptor crosslinking (4) MPX742 peptide 0.01 mM pre-treatment+IgE coating+IgE receptor crosslinking.

### DETAILED DESCRIPTION

Intracellular signal transduction is an orchestrated cascade of events resulting in cell activation. In most eukaryotic cells it is facilitated by a complex protein network mainly composed of phosphatases, kinases and adaptor/regulatory factors. Adaptor molecules play an indispensable role in intracellular signaling pathways by facilitating the interaction of key signaling molecules. Specific domains within adaptor molecules are responsible for facilitating protein interactions. These include Src homology (SH) domains (for example Src homology 2 (SH2) and Src homology 3 (SH3) domains), and pleckstrin homology (PH) domains. During intracellular signaling events, Src homology domains bind to phosphotyrosine-containing proteins, triggering a series of events that leads to the recruitment of pleckstrin homology (PH) domain containing proteins. The recruitment of PH domain containing proteins in turn catalyses the production of secondary messengers facilitating the continuation of the signalling cascade.

As described herein, the expression of a number of different genes encoding SH2/SH3/PH domain containing proteins is altered during mast cell activation. Among the genes determined to have altered expression during mast cell activation are NEDD9 and PHLDA1, each of which is upregulated in activated bone-marrow derived human mast cells compared to resting cells.

The human NEDD9 gene (neural precursor cell-expressed developmentally downregulated gene 9) (GenBank IDs: NM_{—}006403.2 and NM_{—}182966.2, Ensembl ID: ENSG00000111859, Entrez gene ID 4739 and Uniprot ID Q14511), also known as CasL (Crk-associated substrate lymphocyte type) and HEF1 (human enhancer of filamentation 1) encodes the expression of an unprocessed precursor protein of 834 amino acids. NEDD9 is a multifunctional docking protein involved in propagating intracellular signals and is expressed in a wide variety of tissues in the nucleus, cytoplasm and structures such as Golgi and lamellipodia. NEDD9 contains an SH3 domain and a domain rich in SH2-binding sites. These domains facilitate high selectivity interactions with Lck, Lyn and Fyn, three important intracellular players located downstream of surface receptors including T cell, B cell and Fc receptors, as well as other intracellular partners.

PHLDA1 (Pleckstrin homology-like domain, family A, member 1) (GenBank ID: NM_{—}007350, Ensembl gene ID: ENSG00000139289, Entrez gene ID: 22822) encodes the protein PHLDA1 (Uniprot ID: Q8WV24), also known as PHLA1, TDAG51 (T-cell death associated gene 51) and PHRIP (proline and histidine rich protein). PHLDA1 contains a pleckstrin homology (PH) domain. PH domains bind with high affinity to phosphoinositides (phosphorylated derivatives of phosphatidylinositol) which are sequestered to cell membranes. By virtue of binding to phosphoinositides, PH domain containing proteins are generally recruited to the cell membrane where they can then exert their function in cell signaling.

In certain aspects and embodiments, the invention provides peptides capable of modulating the activation of mast cells. The peptides comprise sequences corresponding to at least a portion of an SH3 domain sequence and/or a PH domain sequence. Preferably, the SH3 domain is a NEDD9 SH3 domain. Preferably, the PH domain is a PHLDA1 PH domain. In other aspects and embodiments of the invention, SH2/SH3/PH domain containing proteins are provided that are also capable of modulating mast cell activation.

Without being restricted to a particular mechanism, it is believed that SH2/SH3/PH domain containing proteins (such as NEDD9 and PHLDA1) modulate mast cell activity via the binding of their SH2 domains, SH3 domains, and/or PH domains to specific target proteins with corresponding binding domains in the mast cell interior. Accordingly, SH2/SH3/PH domain containing proteins and peptides of the invention may reduce, enhancing, prevent or otherwise modify the interaction of SH2 domains, SH3 domains, or PH domains with important intracellular players located downstream of mast cell surface receptors such as Lck, Lyn and Fyn. The peptides and SH2/SH3/PH domain containing proteins of the invention may be thus be administered to modulate mast cell activation, providing a means for the treatment and/or prevention of hypersensitivity diseases and disorders.

### SH2/SH3/PH Domain Containing Proteins and Competitor Peptides

Described herein are SH2/SH3/PH domain containing proteins capable of modulating mast cell activation. Proteins capable of modulating mast cell activation in accordance with aspects and embodiments of the invention comprise at least one of an SH2, SH3 or PH domain. Examples of suitable SH2/SH3/PH domain containing proteins include, but are not limited to NEDD9 and PHLDA1.

In the context of the present specification, it will be understood that a NEDD9 protein encompasses all variants and isoforms of that protein which comprise an SH3 domain. Isoforms of the NEDD9 protein include, for example, isoform 1 (Q14511) (REFSEQ accession NM_{—}006403.2), isoform 2 (UniProt: Q5XKI0), isoform CRA_a (UniProt identifier Q5T9R4), and isoform CRA_c (REFSEQ: EAW55302.1). Other NEDD9 protein variants are listed, for example, in the UniProt database including a protein (UniProt: Q5TI59). NEDD9 proteins arising from post-translational modifications such as isoform 1 proteins p115, p105, p65, and p55 are also contemplated.

Similarly, it will be understood that in the context of the present specification a PHLDA1 protein encompasses all variants and isoforms of that protein which comprise a PH domain. Accordingly, a PHLDA1 protein may be encoded by the PHLDA1 gene as described by Entrez Gene ID 22822 (Pleckstrin homology-like domain family A member 1) the expression of which results in a transcript of 5913 nucleotides (see NCBI REFSEQ accession NM_{—}007350) and may be translated, for example into a protein set forth by REFSEQ: NP_{—}031376, REFSEQ: EAW97312.1 or UniProt identifier Q8WV24. The PHLDA1 protein encoded by the variant PHLDA1 CRA_a (e.g. REFSEQ: AAH18929.3, AAI10821.1 and AI26426.2) in the Entrez database is also contemplated.

An SH2/SH3/PH domain containing protein of the invention may have, but is limited to, the polypeptide sequence set forth in SEQ ID NO: 2 or SEQ ID NO: 4. The polynucleotide sequence encoding an SH2/SH3/PH domain containing protein of the invention may be as set forth in SEQ ID NO: 1 or SEQ ID NO: 3, or display sufficient sequence identity thereto to hybridise to the sequences set forth in SEQ ID NO: 1 or SEQ ID NO: 3. In alternative embodiments, the sequence of the polynucleotide encoding an SH2/SH3/PH domain containing protein of the invention may share at least 40%, 50%, 55%, 60%, 65%, 70%, 75%, 80%, 85%, 90%, 95%, 96%, 97%, 98% or 99% identity with the sequence set forth in SEQ ID NO: 1 and/or the sequence set forth in SEQ ID NO: 3.

In accordance with other aspects and embodiments, an SH2/SH3/PH domain containing protein of the invention may have, but is not limited to, the polypeptide sequence set forth in SEQ ID NO: 6. The sequence of the polynucleotide encoding an SH2/SH3/PH domain containing protein of the invention may be as set forth in SEQ ID NO: 5, or display sufficient sequence identity thereto to hybridise to the sequences of SEQ ID NO: 5. In alternative embodiments, the nucleotide sequence of the polynucleotide encoding an SH2/SH3/PH domain containing protein of the invention may share at least 40%, 50%, 55%, 60%, 65%, 70%, 75%, 80%, 85%, 90%, 95%, 96%, 97%, 98% or 99% identity with the sequence set forth in SEQ ID NO: 5.

Described herein are peptides comprising a sequence corresponding to at least a portion of an SH3 domain. Preferably, the SH3 domain is a NEDD9 SH3 domain. Also described herein are peptides comprising a sequence corresponding to at least a portion of a PHLDA1 protein PH domain. The peptides of the invention may be considered to be “competitor peptides” in that their sequence corresponds at least in part to some or all of the NEDD9 SH3 domain, or some or all of the PHLDA1 PH domain. In accordance with certain aspects and embodiments, a peptide of the invention may have a sequence as set forth in SEQ ID NO: 7, SEQ ID NO: 8, SEQ ID NO: 10, SEQ ID NO: 11, SEQ ID NO: 12 and SEQ ID NO: 13.

In accordance with other aspects and embodiments, a peptide of the invention may have a sequence as set forth in any one of SEQ ID NOS: 14-273.

In accordance with other aspects and embodiments, a peptide of the invention may have a sequence as set forth in any one of SEQ ID NOS: 274-488.

In general, the proteins and of the invention are of an isolated or purified form.

It will be understood that included within the scope of the SH2/SH3/PH domain containing proteins and peptides of the invention are variants and fragments thereof. The term “variant” as used herein refers to a substantially similar sequence. In general, two sequences are “substantially similar” if the two sequences have a specified percentage of amino acid residues or nucleotides that are the same (percentage of “sequence identity”), over a specified region, or, when not specified, over the entire sequence. Accordingly, a “variant” of a polynucleotide and polypeptide sequence disclosed herein may share at least 40%, 45%, 50%, 55%, 60%, 65%, 70%, 75%, 80%,83% 85%, 88%, 90%, 93%, 95%, 96%, 97%, 98% or 99% sequence identity with the reference sequence.

In general, sequence variants possess qualitative biological activity in common. Polynucleotide sequence variants generally encode polypeptides which generally possess qualitative biological activity in common. Also included within the meaning of the term “variant” are homologues of SH2/SH3/PH domain containing proteins and competitor peptides of the invention. A homologue is typically from a different family, genus or species sharing substantially the same biological function or activity as the corresponding SH2/SH3/PH domain containing protein or competitor peptide disclosed herein. For example, SH2/SH3/PH domain containing protein or competitor peptide homologues may include, but are not limited to those derived from other different species of mammals.

Further, the term “variant” also includes analogues of the SH2/SH3/PH domain containing proteins and peptides of the invention. A polypeptide “analogue” is a polypeptide which is a derivative of an SH2/SH3/PH domain containing protein or a derivative of a peptide of the invention, which derivative comprises addition, deletion, substitution of one or more amino acids, such that the protein/peptide retains substantially the same function. The term “conservative amino acid substitution” refers to a substitution or replacement of one amino acid for another amino acid with similar properties within a polypeptide chain of an SH2/SH3/PH domain containing protein or a peptide of the invention.

For example, the substitution of the charged amino acid glutamic acid (Glu) for the similarly charged amino acid aspartic acid (Asp) would be a conservative amino acid substitution. Amino acid additions may result from the fusion of a polypeptide of the invention with a second polypeptide or peptide, such as a polyhistidine tag, maltose binding protein fusion, glutathione S transferase fusion, green fluorescent protein fusion, or the addition of an epitope tag such as FLAG or c-myc.

In general, the properties and characteristics of the proteins and peptides described herein may be modified in order to attempt to improve suitability for a particular therapeutic application. Non-limiting examples of such properties and characteristics that may be improved include but are not limited to solubility, chemical and biochemical stability, cellular uptake, toxicity, immunogenicity and excretion of degradation products. Methods and approaches by which the characteristics and properties of the peptides described herein may be improved are well known in the art. For example, one approach is to search for and identify particular amino acid residues that are either negative or positive determinants for a particular property. This maybe achieved, for example, by using the technique of side-chain amputation, in which amino acids are substituted one at a time by the prototypic residue, L-alanine, along the sequence of a peptide. Ascertaining key determinant loci provides a basis for generating and testing variants with both naturally occurring and unnatural amino acid substitutions at the loci identified. Lead peptides that exhibit desirable features may be used as templates for the design of peptidomimetic molecules with improved stability profiles and pharmacokinetic properties. This approach employs structural modifications guided by rational design and molecular modelling. These include but are not limited to conformationally restricted building blocks and peptide bond isosteres (see, for example, Vaguer et al, 2008, “Peptidomimetics, a synthetic tool of drug discover”, Current Opinion in Chemical Biology, 12: 292-296.)

The percentage of sequence identity between two sequences may be determined by comparing two optimally aligned sequences over a comparison window. The portion of the sequence in the comparison window may, for example, comprise deletions or additions (i.e. gaps) in comparison to the reference sequence (for example, a the polynucleotide or polypeptide sequence of an SH2/SH3/PH domain containing protein or peptide disclosed herein), which does not comprise deletions or additions, in order to alignment of the two sequences optimally. A percentage of sequence identity may then be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In the context of two or more nucleic acid or polypeptide sequences, the percentage of sequence identity, refers to the specified percentage of amino acid residues or nucleotides that are the same over a specified region, (or, when not specified, over the entire sequence), when compared and aligned for maximum correspondence over a comparison window, or designated region as measured using one of the following sequence comparison algorithms or by manual alignment and visual inspection.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. Methods of alignment of sequences for comparison are well known in the art. Optimal alignment of sequences for comparison can be determined conventionally using known computer programs, including, but not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). The BESTFIT program (Wisconsin Sequence Analysis Package, Version 8 for Unix, Genetics Computer Group, University Research Park, 575 Science Drive, Madison, Wis. 53711). BESTFIT uses the local homology algorithm of Smith and Waterman to find the best segment of homology between two sequences (Advances in Applied Mathematics 2:482-489 (1981)). When using BESTFIT or any other sequence alignment program to determine the degree of homology between sequences, the parameters may be set such that the percentage of identity is calculated over the full length of the reference sequence and that gaps in homology of up to 5% of the total number of nucleotides or amino acid residues in the reference sequence are allowed.

GAP uses the algorithm described in Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP presents one member of the family of best alignments.

Another method for determining the best overall match between a query sequence and a subject sequence, also referred to as a global sequence alignment, can be determined using the FASTDB computer program based on the algorithm of Brutlag and colleagues (Comp. App. Biosci. 6:237-245 (1990)). In a sequence alignment the query and subject sequences are both DNA sequences. An RNA sequence can be compared by converting U's to T's. The result of said global sequence alignment is in percent identity.

The BLAST and BLAST 2.0 algorithms, may be used for determining percent sequence identity and sequence similarity. These are described in Altschul et al. (1977) Nuc. Acids Res. 25:3389-3402, and Altschul et at (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl, Acad. Sci. USA 89:10915) alignments (B) of 50, expectation (E) of 10, M=5, N=−4, and a comparison of both strands.

The BLAST algorithm also performs a statistical analysis of the similarity between two sequences (see, for example, Karlin and Altschul (1993) Proc. Natl. Acad. Sd. USA 90:5873- 5787). One measure of similarity provided by the BLAST algorithm is 5 the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The invention also contemplates fragments of the SH2/SH3/PH domain containing proteins and peptides of the invention. A “fragment” is a polypeptide molecule that encodes a constituent or is a constituent of an SH2/SH3/PH domain containing protein and/or peptide of the invention or variant thereof. Typically the fragment possesses qualitative biological activity in common with the SH2/SH3/PH domain containing protein and/or peptide of which it is a constituent. The peptide fragment may be between about 3 to about 2000 amino acids in length, between about 3 to about 1750, between about 3 to about 1500 amino acids in length, between about 3 to about 1250 amino acids in length, between about 3 to about 1000 amino acids in length, between about 3 to about 950 amino acids in length, between about 3 to about 900 amino acids in length, between about 3 to about 850 amino acids in length, between about 3 to about 800 amino acids in length, between about 3 to about 750 amino acids in length, between about 3 to about 700 amino acids in length, between about 3 to about 650 amino acids in length, between about 3 to about 600 amino acids in length, between about 3 to about 550 amino acids in length, between about 3 to about 500 amino acids in length, between about 3 to about 450 amino acids in length, between about 3 to about 400 amino acids in length, between about 3 to about 350 amino acids in length, between about 3 to about 300 amino acids in length, between about 3 to about 250 amino acids in length, between about 3 to about 200 amino acids in length, between about 3 to about 150 amino acids in length, between about 3 to about 125 amino acids in length, between about 3 to about 100 amino acids in length, between about 3 to about 75 amino acids in length, between about 3 to about 50 amino acids in length, between about 3 to about 40 amino acids in length, between about 3 to about 35 amino acids in length, between about 3 to about 30 amino acids in length, between about 3 to about 25 amino acids in length, between about 3 to about 20 amino acids in length, between about 3 to about 15 amino acids in length, between about 3 to about 10 amino acids in length, between about 3 to about 7 amino acids in length, between about 5 to about 10 amino acids in length, between about 5 to about 15 amino acids in length, between about 5 to about 20 amino acids in length, between about 5 to about 25 amino acids in length, between about 5 to about 30 amino acids in length, between about 5 to about 35 amino acids in length, between about 8 to about 12 amino acids in length, between about 8 to about 15 amino acids in length, between about 8 to about 20 amino acids in length, between about 8 to about 25 amino acids in length, and between about 8 to about 30 amino acids in length.

Also contemplated are fragments of the polynucleotides disclosed herein. A polynucleotide “fragment” is a polynucleotide molecule that encodes a constituent or is a constituent of a polynucleotide of the invention or variant thereof. Fragments of a polynucleotide may or may not encode an SH2/SH3/PH domain containing protein or peptide which retains biological activity. A biologically active fragment of an SH2/SH3/PH domain containing protein or peptide used in accordance with the present invention may typically possess at least about 50% of the immunomodulatory activity of the corresponding full length protein, more typically at least about 60% of such activity, more typically at least about 70% of such activity, more typically at least about 80% of such activity, more typically at least about 90% of such activity, and more typically at least about 95% of such activity. The fragment may, for example, be useful as a hybridization probe or PCR primer. The fragment may be derived from a polynucleotide of the invention or alternatively may be synthesized by some other means, for example chemical synthesis.

Variants of the SH2/SH3/PH domain containing proteins and peptides of the invention can be generated by mutagenesis. Mutagenesis may be directed at the SH2/SH3/PH domain containing protein or peptides of the invention, or, an encoding nucleic acid, such as by random mutagenesis or site-directed mutagenesis using methods well known to those skilled in the art. Such methods are described, for example in Current Protocols In Molecular Biology (Chapter 9), Ausubel et al, 1994, John Wiley & Sons, Inc., New York, the disclosure of which is incorporated herein by reference. Variants and analogues as described herein also encompass polypeptides complexed with other chemical moieties, fusion proteins or otherwise post-transitionally modified. Further, the SH2/SH3/PH domain containing proteins, peptides and fragments or variants thereof may possess other post-translational modifications, including side-chain modifications such as for example acetylation, amidination, carbamoylation, reductive alkylation and other modifications as are known to those skilled in the art.

SH2/SH3/PH domain containing proteins, peptides and variants or fragments thereof may be obtained using any suitable method known in the art. For example, SH2/SH3/PH domain containing proteins, peptides of the invention, and variants or fragments thereof may be obtained, using standard recombinant nucleic acid techniques or may be synthesized, for example, using conventional liquid or solid phase synthesis techniques. SH2/SH3/PH domain containing proteins and peptides of the invention may be produced, for example, by digestion of a polypeptide with one or more proteinases such as endoLys-C, endoArg-C, endoGlu-C and staphylococcus V8-protease. The digested peptide fragments can be purified by, for example, high performance liquid chromatographic (HPLC) techniques. Recombinant protein production techniques will typically involve the cloning of a gene encoding an SH2/SH3/PH domain containing protein or a sequence encoding a peptide described herein into a plasmid for subsequent overexpression in a suitable microorganism.

Suitable methods for the construction of expression vectors or plasmids are described in detail, for example, in standard texts such as Sambrook et al., Molecular Cloning: A Laboratory Manual, Cold Spring Harbor, New York, 1989, and Ausubel et al., Current Protocols in Molecular Biology, John Wiley and Sons, copyright 2007. Methods for producing the recombinant SH2/SH3/PH domain containing proteins and polypeptides of the invention are described in detail, for example, in standard texts such as Coligan et al., Current Protocols in Protein Science, (Chapter 5), John Wiley and Sons, Inc., copyright 2007, and Pharmacia Biotech., The Recombinant Protein Handbook 1994, Pharmacia Biotech.

Commonly used expression systems that may be used for the production of SH2/SH3/PH domain containing proteins and peptides of the invention include, for example, bacterial (e.g. E. coli), yeast (e.g. Saccharomyces cerevisiae Aspergillus, Pichia pastorisis), viral (e.g. baculovirus and vaccinia), cellular (e.g. mammalian and insect) and cell-free systems. Cell-free systems may be also used including eukaryotic rabbit reticuloctye, wheat germ extract systems, and the prokaryotic E. coli cell-free system, using methods described in, for example, Madin et al., Proc. Natl. Acad. Sci. U.S.A. 97:559-564 (2000), Pelham and Jackson, Eur. J. Biochem., 67: 247-256 (1976), Roberts and Paterson, Proc. Natl. Acad. Sci., 70: 2330-2334 (1973), Zubay, Ann. Rev. Genet., 7: 267 (1973), Gold and Schweiger, Meth. Enzymol., 20: 537 (1971), Lesley et al., J. Biol. Chem., 266(4): 2632-2638 (1991), Baranov et al., Gene, 84: 463-466 (1989) and Kudlicki et al., Analyt. Biochem., 206: 389-393 (1992).

SH2/SH3/PH domain containing proteins and competitor peptides of the invention (along with fragments and variants of each) may be synthesised using standard methods of liquid and solid phase chemistry well known in the art (see for example, Hackeng et al., Proc Natl Acad Sci USA. 96(18):10068-73 (1999), and Steward and Young, Solid Phase Peptide Synthesis (2nd Edn.), Pierce Chemical Co., Illinois, USA (1984). In general, this synthesis method comprises the sequential addition of one or more amino acids or suitably protected amino acids to a growing peptide chain. Typically, either the amino or carboxyl group of the first amino acid is protected by a suitable protecting group. The protected amino acid is then either attached to an inert solid support or utilised in solution by adding the next amino acid in the sequence having the complimentary (amino or carboxyl) group suitably protected and under conditions suitable for forming the amide linkage. The protecting group is then removed from this newly added amino acid residue and the next (protected) amino acid is added, and so forth. After all the desired amino acids have been linked, any remaining protecting groups, and if necessary any solid support, is removed sequentially or concurrently to produce the final polypeptide

Changes to the amino acid sequence of SH2/SH3/PH domain containing proteins and peptides of the invention may be affected by standard techniques in the art. For example, amino acid changes may be effected by nucleotide replacement techniques which include the addition, deletion or substitution of nucleotides (conservative and/or non-conservative), under the proviso that the proper reading frame is maintained. Exemplary techniques include random mutagenesis, site-directed mutagenesis, oligonucleotide-mediated or polynucleotide-mediated mutagenesis, deletion of selected region(s) through the use of existing or engineered restriction enzyme sites, and the polymerase chain reaction. Testing of immunomodulatory activity for the purposes of the present invention may be via any one of a number of techniques known to those of skill in the art.

Purification of SH2/SH3/PH domain containing proteins and peptides of the invention (along with fragments and variants of each) may be achieved using standard techniques in the art such as those described in Coligan et al., Current Protocols in Protein Science, (Chapter 6), John Wiley and Sons, Inc., copyright 2007. For example, if the protein or peptide is in a soluble state, it may be isolated using standard methods such as column chromatography. SH2/SH3/PH domain containing proteins and peptides described herein may be genetically engineered to contain various affinity tags or carrier proteins that aid purification. For example, the use of histidine and protein tags engineered into an expression vector containing the SH2/SH3/PH domain containing protein and/or peptide of the invention may facilitate purification by, for example by metal-chelate chromatography (MCAC) under either native or denaturing conditions. Purification may be scaled-up for large-scale production purposes.

The skilled addressee will appreciate that the invention is not limited by the method of production or purification utilised, the methods and techniques described above being provided for the purpose of exemplification only.

### Immune Response Modulation and Inhibition of Mast Cell Activation

Described herein is a method of modulating an immune response in a subject. The method comprises administering to the subject a therapeutically effective amount of a protein comprising at least one of

(i) an Src homology 2 (SH2) domain,

(ii) an Src homology 3 (SH3) domain,

(iii) a pleckstrin homology (PH) domain,

(iv) a peptide of the invention.

In one embodiment, the protein is NEDD9 or a fragment or variant thereof. In another embodiment, the protein is PHLDA1 or a fragment or variant thereof.

The immune response may be enhanced or inhibited by the methods of the invention. For example, the administration an effective amount of an SH2/SH3/PH domain containing protein or a peptide as described herein that negatively regulates the mast cell activation pathway may result in the suppression of mast cell activation. Consequently, a hypersensitivity response within the subject may be reduced or prevented following administration.

Alternatively, the administration an effective amount of an SH2/SH3/PH domain containing protein domain containing protein or a peptide as described herein that positively regulates the mast cell activation may result in an enhancement of mast cell activation. Consequently, a hypersensitivity response within the subject may be increased following administration.

Also provided herein are methods of inhibiting or preventing mast cell activation. In certain aspects of the invention, the methods of inhibiting or preventing mast cell activation comprise the administration of a protein comprising at least one of:

(i) an Src homology 2 (SH2) domain,

(ii) an Src homology 3 (SH3) domain,

(iii) a pleckstrin homology (PH) domain,

(iv) a peptide of the invention.

Preferably, the protein is NEDD9 or PHLDA1.

In other aspects of the invention, the methods of inhibiting or preventing mast cell activation comprises the administration of a therapeutically effective amount of a peptide, the peptide comprising an amino acid sequence corresponding to at least a portion of:

(i) an Src homology 3 (SH3) domain, or

(ii) a pleckstrin homology (PH) domain.

Preferably, the sequence of the peptide corresponds at least in part to the NEDD9 Src homology 3 (SH3) domain. Preferably, the sequence of the peptide corresponds at least in part to PHLDA1 pleckstrin homology (PH) domain.

In certain embodiments of the invention, the peptide corresponding at least in part to the NEDD9 Src homology 3 (SH3) domain may have the amino acid sequence set forth in SEQ ID NO: 7 or SEQ ID NO 8. In other embodiments of the invention, the peptide corresponding at least in part to the NEDD9 Src homology 3 (SH3) domain may have the amino acid sequence set forth in any one of in SEQ ID NOS: 14-273.

In certain embodiments of the invention, the peptide corresponding at least in part to the PHLDA1 pleckstrin homology (PH) domain may have the amino acid sequence set forth in SEQ ID NO: 10, SEQ ID NO: 11 or SEQ ID NO: 12. In other embodiments of the invention, the peptide corresponding at least in part to the PHLDA1 pleckstrin homology (PH) domain may have the amino acid sequence set forth in SEQ ID NO: 13, or any one of SEQ ID NOs: 274-488.

The mast cell activation may arise through IgE dependant mechanisms, such as by IgE crosslinking on the mast cell surface. Additionally or alternatively, the mast cell activation may arise from IgE-independent mechanisms.

Measurement and comparison of the level of activation in mast cells that have or have not been administered an SH2/SH3/PH domain containing protein or peptide as described herein may be performed using methods known in the art. For example, the level of mast cell activation can be measured by standard assays which detect factors released upon the degranulation of activated mast cells. These factors may include, for example, histamine, tryptase, serine protease tryptase, β-hexosaminidase, heparin, chondroitin sulphate E, prostaglandin D2, Leukotriene B4 and C4, platelet activation factor, or cytokine release including IL-3, IL-4, IL-5, IL-6, IL-8, IL-10, IL-13, GM-CSF, TNF, CCL2, CCL3 and CCL5. Additionally or alternatively, the degree of mast cell activation may be measured by changes in the expression of specific markers of mast cell activation, for example, by flow cytometry or immunocytochemistry. Mast cells may be identified using such techniques by the expression of various surface receptors known in the art to be expressed by mast cells, including but not limited to CD9, CD29, CD33, CD43, CD44, CD45, CD46, CD51, CD54, CD55, CD58, CD59, CD61, and CD117, CD47, CD48, CD49d, CD53, CD60, CD63, CD81, CD82, CD84, CD87, CD92, CD97, CD98, and CD99, CD147, CD149, CD151, and CD157. The level of activation in the mast cells so identified may be measured, for example, by the expression of a mast cell activation marker, for example CD107A (LAMP-1), CD107B (LAMP-2), tryptase and PGD2.

As described herein in the accompanying Examples, it has been determined that the administration of “competitor peptides” corresponding in sequence to specific regions within the NEDD9 SH3 or PHLDA1 PH domains have the effect of desensitising mast cells and inhibiting their activation upon IgE crosslinking. Without wishing to be restricted to a particular mechanism, it is speculated that peptides corresponding to NEDD9 SH3 exhibit this effect on mast cell activation by interfering with the normal binding of the NEDD9 SH3 domain with SH3 binding domains of target proteins in the mast cell interior. This is thought to alter high selectivity interactions with Lck, Lyn and Fyn, three important intracellular players located downstream of these mast cell surface receptors, thereby disrupting the activation pathway. Similarly, it is speculated that peptides corresponding to the PHLDA1 PH domain inhibit mast cell activation by interfering with the normal binding of the PHLDA1 PH domain with PH binding domains present in target proteins within mast cells.

The present inventors have demonstrated that interfering with the binding of the NEDD9 SH3 domain and/or the PHLDA1 PH domain to target proteins in mast cells provides a means of modulating mast cell activation. In addition to the peptides exemplified, a large number of peptides based on the NEDD9 SH3 domain or PHLDA1 PH domain sequences can be designed utilised, and it is to be understood that these are included within the scope of the invention.

The invention also contemplates agents which may exert their modulatory effect on the immune response of the subject by altering the expression of an SH2/SH3/PH domain containing protein. In this case, such agents may be identified by comparing the expression of SH2/SH3/PH domain containing protein in the presence of the candidate agent with the level of expression of SH2/SH3/PH domain containing protein in the absence of the candidate agent. The expression of a gene encoding an SH2/SH3/PH domain containing protein may be increased by the agent, for example, by contacting a regulatory sequence of the gene and thereby increase its transcription. Alternatively, the expression of a gene encoding SH2/SH3/PH domain containing protein may be reduced or inhibited by the agent, for example, by binding the gene in such a way or manner that the access of a protein involved in the transcription machinery of the gene is hindered or prevented from functioning.

In addition, modulatory effects on the immune response and mast cell activation may be achieved by reducing or inhibiting the production of an SH2/SH3/PH domain containing protein such as NEDD9 or PHLDA1 by the administration of a homologous antisense nucleic acid. Therapeutic or prophylactic use of such nucleic acids of at least 5 nucleotides, generally up to about 200 nucleotides, that are antisense to a gene of complementary DNA (cDNA) encoding the SH2/SH3/PH domain containing protein (such as NEDD9 or PHLDA1) is also provided herein. Such an antisense nucleic acid may be capable of hybridising to a portion of the RNA precursor (generally mRNA) of an SH2/SH3/PH domain containing protein, by virtue of some sequence complementarity, and generally under high stringency conditions. The antisense nucleic acid may be complementary to a coding and/or non-coding region of the RNA precursor of the SH2/SH3/PH domain containing protein. Absolute complementarily to the full RNA precursor is not required. Antisense nucleic acids in this form have utility as therapeutics that reduce or inhibit mast cell activation, and can be used in the treatment or prevention of disease states as described herein.

The antisense nucleic acids complementary to the RNA precursor of the SH2/SH3/PH domain containing protein, such as NEDD9 or PHLDA1 , may be of at least five nucleotides and are generally oligonucleotides which range in length from 5 to about 200 nucleotides. For example, the anti-sense oligonucleotide is at least 10 nucleotides, at least 15 nucleotides, at least 100 nucleotides, at least 125 nucleotides, at least 150 nucleotides, or at least 175 nucleotides. The oligonucleotides can be DNA or RNA or chimeric mixtures or derivatives or modified versions thereof, single-stranded or double-stranded.

The anti-sense nucleic acid complementary to the RNA precursor can be modified at any position on its structure using substituents generally known in the art. The anti-sense nucleic acid may include at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, 2,2-dimethylguanine, 2-methyl-adenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, pseudouracil, 2-thiocytosine, 5-methyl-2-thiouracil, 2-thiouracil, 4-thiouracil, 5-methyluracil, uracil-5-oxyacetic acid methylester, uracil-5-oxyacetic acid (v), queosine, wybutoxosine, 5-methyl-2-thiouracil, 3-(3-amino-3-N-2-carboxypropyl) uracil, and 2,6-diaminopurine.

The anti-sense nucleic acid complementary to the RNA precursor may include at least one modified sugar moiety, such as arabinose, 2-fluoroarabinose, xylulose, and hexose. The antisense nucleic acid may also include at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analogue thereof. The anti-sense nucleic acid can be conjugated to another molecule, such as a peptide, hybridisation triggered cross-linking agent, transport agent or a hybridisation-triggered cleavage agent.

Expression of the sequence encoding anti-sense nucleic acid complementary to the RNA precursor of the SH2/SH3/PH domain containing protein (e.g. NEDD9 or PHLDA1) can be by any promoter known in the art to act in mammalian, including human, cells, and may include inducible or constitutive promoters.

RNA interference (RNAi) (see, for example, Chuang et al., Proc Natl Acad Sci USA 97: 4985-4990 (2000)) can be employed to inhibit the expression of a gene encoding an SH2/SH3/PH domain containing protein such as NEDD9 or PHLDA1. Interfering RNA (RNAi) fragments, particularly double-stranded RNAi, can be used to cause loss of the protein. Methods relating to the use of RNAi to silence genes in organisms are known, for instance, Fire et al., Nature 391: 806-811 (1998); Hammond et al., Nature Rev, Genet. 2: 110-1119 (2001); Hammond et al., Nature 404: 293-296 (2000); Bernstein et al., Nature 409: 363-366 (2001); Elbashir et al., Nature 411: 494-498 (2001); International PCT application No. WO 01/29058; and International PCT application No. WO 99/32619), the disclosures of which are incorporated herein by reference.

Double-stranded RNA expressing constructs are introduced into a host using a replicable vector that remains episomal or integrates into the genome. By selecting appropriate sequences, expression of dsRNA can interfere with accumulation of endogenous mRNA encoding an IL-10 homologue.

Treatment and/or Prevention of Hypersensitivity

In one aspect, the invention relates to a method for inhibiting or preventing a hypersensitivity disease or disorder. The method comprises the step of administering a therapeutically effective amount of a protein comprising at least one of:

(i) an Src homology 2 (SH2) domain,

(ii) an Src homology 3 (SH3) domain,

(iii) a pleckstrin homology (PH) domain,

(iv) a peptide of the invention.

Preferably, the protein is NEDD9 or a fragment or variant thereof. Preferably, the the protein is PHLDA1 or a fragment or variant thereof.

Also described herein is a method of treating or preventing a hypersensitivity disease or disorder comprising administering a therapeutically effective amount of a peptide comprising an amino acid sequence corresponding to at least a portion of:

(i) an Src homology 3 (SH3) domain, or

(ii) a pleckstrin homology (PH) domain.

Preferably, the protein is NEDD9 or a fragment or variant thereof. Preferably, the the protein is PHLDA1 or a fragment or variant thereof.

Preferably, the peptide comprises an amino acid sequence selected from the group consisting of SEQ ID NO: 7, SEQ ID NO: 8, SEQ ID NO: 10, SEQ ID NO: 11, SEQ ID NO: 12, and variants and fragments thereof.

In one embodiment, the peptide comprises an amino acid set forth in SEQ ID NOs: 14-273.

In another embodiment the peptide comprises an amino acid set forth in any one of SEQ ID NOs: 274-488.

Also provided herein is a method for treating or preventing a hypersensitivity disease or disorder comprising inhibiting the binding of either of both of:

(i) a NEDD9 protein Src homology 3 (SH3) domain

(ii) a PHLDA1 protein pleckstrin homology (PH) domain

to a mast cell receptor.

The inhibition of binding may comprise the administration of a peptide comprising an amino acid sequence corresponding to at least a portion of the NEDD9 protein Src homology 3 (SH3) domain or the PHLDA1 protein pleckstrin homology (PH) domain.

Preferably, the peptide comprises an amino acid sequence selected from the group consisting of SEQ ID NO: 7, SEQ ID NO: 8, SEQ ID NO: 10, SEQ ID NO: 11, SEQ ID NO: 12, and variants and fragments thereof.

In one embodiment, the peptide comprises an amino acid set forth in SEQ ID NOs: 14-273.

In another embodiment the peptide comprises an amino acid set forth in any one of SEQ ID NOs: 274-488.

The hypersensitivity disease or disorder may arise wholly or partially from mast cell activation. The mast cell activation may occur via IgE dependent or IgE independent mechanisms. The hypersensitivity disease or disorder may comprise an inflammatory reaction.

Examples of hypersensitivity diseases or disorders that may be prevented or treated in accordance with the methods described herein include, but are not limited to anaphylaxis, drug reactions, skin allergy, eczema, allergic rhinitis, urticaria, atopic dermatitis, allergic contact allergy, food allergy, allergic conjunctivitis, insect venom allergy and respiratory diseases and disorders such as asthma, allergic asthma, intrinsic asthma, occupational asthma, acute respiratory distress syndrome (ARDS) and chronic obstructive pulmonary disease (COPD).

The methods of treating or preventing hypersensitivity diseases or disorders may further comprise the administration of at least one additional agent. The additional agent may be an immunomodulator, which, in the context of the invention, is a molecular mediator secreted by one or more cell types and which plays a role in the activation, maintenance, maturation, inhibition, suppression or augmentation of an immune response. In another embodiment, the immunomodulator may be a type I interferon.

In accordance with aspects and embodiments of the invention, a subject in need of treatment may be administered with an effective amount of an SH2/SH3/PH domain containing protein such as NEDD9 or PHLDA1 , or a peptide (for example, SEQ ID NO: 7, SEQ ID NO: 8, SEQ ID NO: 10, SEQ ID NO: 11, SEQ ID NO: 12). Those of ordinary skill in the art will appreciate that the SH2/SH3/PH domain containing protein may be administered in a species-specific manner, such that the protein may be derived from the species to be treated.

The proteins and peptides of the invention may be administered to a subject in the form of a composition. In general, suitable compositions for use in accordance with the methods of the invention may be prepared according to methods which are known to those of ordinary skill in the art and accordingly may include a pharmaceutically acceptable carrier, diluent and/or adjuvant. Compositions of the invention may be prepared comprising an SH2/SH3/PH domain containing protein or a peptide alone, mixtures of different SH2/SH3/PH domain containing proteins, mixtures of peptides, and combinations of SH2/SH3/PH domain containing proteins and peptides are also contemplated. Such compositions may be included in pharmaceutical compositions comprising a pharmaceutically acceptable carrier, adjuvant and/or diluent. Alternatively, the compositions of the invention may also comprise an immunosuppressive agent.

Embodiments of the invention also contemplate the administration of polynucleotides encoding SH2/SH3/PH domain containing proteins (such as NEDD9 or PHLDA1) and/or peptides comprising an amino acid sequence corresponding to at least a portion of an SH3 or PH domain. In such situations the polynucleotide is typically operably linked to a promoter such that the appropriate polypeptide sequence is produced following administration of the polynucleotide to the subject. The polynucleotide may be administered to subjects in a vector. The vector may be a plasmid vector, a viral vector, or any other suitable vehicle adapted for the insertion of foreign sequences, their introduction into eukaryotic cells and the expression of the introduced sequences. The nucleic acid construct to be administered may comprise naked DNA or may be in the form of a composition, together with one or more pharmaceutically acceptable carriers.

Typically the vector is a eukaryotic expression vector and may include expression control and processing sequences such as a promoter, an enhancer, ribosome binding sites, polyadenylation signals and transcription termination sequences. The expression of a gene encoding an SH2/SH3/PH domain containing protein such as NEDD9 or PHLDA1, or a peptide of the invention may be increased in the mast cells of a subject using various methods of gene delivery known in the art. For example, an expression vector comprising a nucleic acid sequence encoding an SH2/SH3/PH domain containing protein regulator of a mast cell activation pathway operably linked to an expression control sequence such as an inducible promoter may be administered to a subject to increase the production of said protein in mast cells. Alternatively, viral vectors (for example retroviral and adenoviral vectors) containing a nucleic acid sequence encoding an SH2/SH3/PH domain containing protein and/or peptide of the invention may be administered to a subject in order to elicit the production of said protein. The delivery of a gene encoding an SH2/SH3/PH domain containing protein or peptide described herein may also be achieved by extracting cells from a subject, administering a vector containing the gene of interest, and then re-introducing the cells to the subject. The expression of the gene encoding an SH2/SH3/PH domain containing protein or peptide of the invention by gene delivery techniques can be used as a means to modulate the activation of mast cells in a subject. Accordingly, such methods may also be suitable for the treatment and prevention of hypersensitivity disease or disorder in a subject.

### Screening for Modulators

The invention also contemplates the use of agonists and antagonists of SH2/SH3/PH domain containing proteins to modulate the immune response of a subject, and provides methods for identifying such agonists and antagonists. Agonists and antagonists of the SH2/SH3/PH domain containing proteins of the invention may be specifically designed or screened according to their effect upon mast cell activation.

In one aspect, the invention relates to a method of identifying an agent that modulates the activity of a protein comprising at least one of an Src homology 2 SH2) domain, an Src homology 3 (SH3) domain, or a pleckstrin homology (PH) domain. The method comprises contacting a candidate agent with the protein under conditions suitable to permit interaction of the candidate agent with the protein and then assaying the activity of the protein.

In another aspect, the invention relates to a method for screening a plurality of candidate agents to identify an agent that modulates the activity of a protein comprising at least one of an Src homology 2 (SH2) domain, an Src homology 3 (SH3) domain, or a pleckstrin homology (PH) domain. The method comprises contacting a plurality of candidate agents with the protein under conditions suitable to permit interaction of the candidate agent with the protein and assaying the activity of the protein.

In a further aspect, the invention relates to a method for screening a plurality of candidate agents to identify an agent that modulates the activity of an SH2/SH3/PH domain containing protein regulator of a mast cell activation pathway. The method comprises the steps of contacting a plurality of candidate agents with an SH2/SH3/PH domain containing protein regulator of a mast cell activation pathway under conditions suitable to permit interaction of the candidate agent with the SH2/SH3/PH domain containing protein regulator of a mast cell activation pathway, and assaying the activity of said SH2/SH3/PH domain containing protein regulator of a mast cell activation pathway.

Preferably, the SH2/SH3/PH domain containing protein referred to in the methods recited above is NEDD9 or PHLDA1.

A variety of suitable methods may be used to determine whether a candidate agent or plurality of candidate agents interacts or binds with an SH2/SH3/PH domain containing protein such as NEDD9 or PHLDA1. Non limiting methods include the two-hybrid method, co-immunoprecipitation, affinity purification, mass spectroscopy, tandem affinity purification, phage display, label transfer, DNA microarrays/gene coexpression and protein microarrays.

For example, a two-hybrid assay may be used to determine whether a candidate agent or plurality of candidate agents interacts or binds with an SH2/SH3/PH domain containing protein of the invention. The yeast two-hybrid assay system is a yeast-based genetic assay typically used for detecting protein-protein interactions (Fields and Song., Nature 340: 245-246 (1989)). The assay makes use of the multi-domain nature of transcriptional activators. For example, the DNA-binding domain of a known transcriptional activator may be fused to the SH2/SH3/PH domain containing protein of the invention and the activation domain of the transcriptional activator fused to the candidate agent. Interaction between the candidate agent and the SH2/SH3/PH domain containing protein will bring the DNA-binding and activation domains of the transcriptional activator into close proximity. Subsequent transcription of a specific reporter gene activated by the transcriptional activator allows the detection of an interaction.

In a modification of the technique above, a fusion protein may be constructed by fusing the SH2/SH3/PH domain containing protein of the invention with a detectable tag, for example, alkaline phosphatase, and using a modified form of immunoprecipitation as described by Flanagan and Leder (Flanagan and Leder, Cell 63:185-194 (1990))

Alternatively, co-immunoprecipation may be used to to determine whether a candidate agent or plurality of candidate agents interacts or binds with an SH2/SH3/PH domain containing protein of the invention. Using this technique, activated mast cells may be lysed under nondenaturing conditions suitable for the preservation of protein-protein interactions. The resulting solution can then be incubated with an antibody specific for an SH2/SH3/PH domain containing protein of the invention and immunoprecipitated from the bulk solution, for example by capture with an antibody-binding protein attached to a solid support. Immunoprecipitation of the SH2/SH3/PH domain containing protein by this method facilitates the co-immunoprecipation of an agent associated with that protein. The identification an associated agent can be established using a number of methods known in the art, including but not limited to SDS-PAGE, western blotting, and mass spectrometry.

Alternatively, the phage display method may be used to to determine whether a candidate agent or plurality of candidate agents interacts or binds with an SH2/SH3/PH domain containing protein of the invention. Phage display is a test to screen for protein interactions by integrating multiple genes from a gene bank into phage. Under this method, recombinant DNA techniques are used to express numerous genes as fusions with the coat protein of a bacteriophage such the peptide or protein product of each gene is displayed on the surface of the viral particle. A whole library of phage-displayed peptides or protein products of interest can be produced in this way. The resulting libraries of phage-displayed peptides or protein products may then be screened for the ability to bind an SH2/SH3/PH domain containing protein of the invention. DNA extracted from interacting phage contains the sequences of interacting proteins.

Alternatively, affinity chromatography may be used to to determine whether a candidate agent or plurality of candidate agents interacts or binds with an SH2/SH3/PH domain containing protein of the invention. For example, an SH2/SH3/PH domain containing protein may be immobilised on a support (such as sepharose) and cell lysates passed over the column. Proteins binding to the immobilised SH2/SH3/PH domain containing protein may then be eluted from the column and identified, for example by N-terminal amino acid sequencing.

Methods for determining whether the interaction or binding of a candidate agent to an SH2/SH3/PH domain containing protein of the invention modulates the activity said protein may be determined by measuring the degree of mast cell activation after stimulation. For example, the level of activation in mast cells administered the candidate agent may be measured and compared to the level of activation in mast cells not administered the candidate agent.

The uptake of a candidate agent into a mast cell may occur by natural diffusion, or may be induced by various methods known in the art, including but not limited to microinjection, electroporation, fusion of the protein with one or more viral protein transduction domains (PTDs), and cationic lipid delivery. Alternatively, production of the candidate agent may be induced within the mast cell, for example, by introducing a plasmid expression vector or virus containing a gene encoding the candidate agent into the mast cell. Methods of transfecting cells with plasmids are well known in the art and include, for example, calcium phosphate coprecipitation, DEAE dextran facilitated transfection, electroporation, microinjection and cationic liposomes.

Mast cell activation may then be induced by a variety of methods, for example, by incubation with IgE followed by crosslinking with DNP/albumin. Various other IgE independent triggers of mast cell activation may also be used, for example, activation may be induced via the FcγR, Toll-like receptors (TLRs) or by the administration of ionomycyin.

Measurement and comparison of the level of activation in mast cells that have or have not been administered a candidate agent may be achieved by methods known in the art. The level of mast cell activation can be measured by standard assays which detect factors released upon the degranulation of activated mast cells, for example, histamine, tryptase, serine protease tryptase, β-hexosaminidase, heparin, chondroitin sulphate E, prostaglandin D2, Leukotriene B4 and C4, platelet activation factor, or cytokine release including IL-3, IL-4, IL-5, IL-6, IL-8, IL-10, IL-13, GM-CSF, TNF, CCL2, CCL3 and CCL5. Alternatively, the degree of mast cell activation may be measured by changes in the expression of specific markers of mast cell activation, for example, by flow cytometry or immunocytochemistry. Mast cells may be identified using such techniques by the expression of various surface receptors known in the art to be expressed by mast cells, including but not limited to CD9, CD29, CD33, CD43, CD44, CD45, CD46, CD51, CD54, CD55, CD58, CD59, CD61, and CD117, CD47, CD48, CD49d, CD53, CD60, CD63, CD81, CD82, CD84, CD87, CD92, CD97, CD98, and CD99, CD147, CD149, CD151, and CD157. The level of activation in the mast cells so identified could be measured, for example, by the expression of a mast cell activation marker, for example CD107A (LAMP-1), CD107B (LAMP-2), tryptase and PGD2.

It will be appreciated that the methods described above are merely examples of the types of methods that may be utilised to identify agents that are capable of interacting with, or modulating the activity of SH2/SH3/PH domain containing proteins of the invention (such as NEDD9 and PHLDA1) described herein. Other suitable methods will be known by persons skilled in the art and are within the scope of this invention.

Using the methods described above, an agent may be identified that is an agonist of an SH2/SH3/PH domain of the invention. Agents which are agonists enhance one or more of the biological activities of an SH2/SH3/PH domain containing protein of the invention. Alternatively, the methods described above may identify an agent that is an antagonist of an SH2/SH3/PH domain containing protein of the invention. Agents which are antagonists retard one or more of the biological activities of an SH2/SH3/PH domain of the invention.

Potential modulators of the activity of an SH2/SH3/PH domain containing protein such as NEDD9 or PHLDA1 may be generated for screening by the above methods by a number of techniques known to those skilled in the art. For example, methods such as X-ray crystallography and nuclear magnetic resonance spectroscopy may be used to model the structure of SH2/SH3/PH domain containing protein of the invention, thus facilitating the design of potential modulating agents using computer-based modelling. Various forms of combinatorial chemistry may also be used to generate putative modulators.

Antibodies may act as agonists or antagonists of SH2/SH3/PH domain containing protein of the invention. Preferably suitable antibodies are prepared from discrete regions or fragments of the SH2/SH3/PH domain containing protein polypeptide. An antigenic SH2/SH3/PH domain containing polypeptide contains at least about 5, and preferably at least about 10, amino acids.

Methods for the generation of suitable antibodies will be readily appreciated by those skilled in the art. For example, a monoclonal antibody specific for an SH2/SH3/PH domain containing protein or a peptide of the invention, typically containing Fab portions, may be prepared using the hybridoma technology described in Antibodies-A Laboratory Manual, Harlow and Lane, eds., Cold Spring Harbor Laboratory, N.Y. (1988).

In essence, in the preparation of monoclonal antibodies directed toward SH2/SH3/PH domain containing proteins or peptides of the invention, any technique that provides for the production of antibody molecules by continuous cell lines in culture may be used. These include the hybridoma technique originally developed by Kohler et al., Nature, 256:495-497 (1975), as well as the trioma technique, the human B-cell hybridoma technique (Kozbor et al., Immunology Today, 4:72 (1983)), and the EBV-hybridoma technique to produce human monoclonal antibodies (Cole et al., in Monoclonal Antibodies and Cancer Therapy, pp. 77-96, Alan R. Liss, Inc., (1985)). Immortal, antibody-producing cell lines can be created by techniques other than fusion, such as direct transformation of B lymphocytes with oncogenic DNA, or transfection with Epstein-Barr virus. See, for example, M. Schreier et al., “Hybridoma Techniques” Cold Spring Harbor Laboratory, (1980); Hammerling et al., “Monoclonal Antibodies and T-cell Hybridomas” Elsevier/North-Holland Biochemical Press, Amsterdam (1981); Kennett et al., “Monoclonal Antibodies”, Plenum Press (1980).

In summary, a means of producing a hybridoma from which the monoclonal antibody is produced, a myeloma or other self-perpetuating cell line is fused with lymphocytes obtained from the spleen of a mammal hyperimmunised with a recognition factor-binding portion thereof, or recognition factor, or an origin-specific DNA-binding portion thereof. Hybridomas producing a monoclonal antibody useful in practicing this invention are identified by their ability to immunoreact with the present recognition factors and their ability to inhibit specified transcriptional activity in target cells.

A monoclonal antibody useful in practicing the invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques.

Similarly, there are various procedures known in the art which may be used for the production of polyclonal antibodies. For the production of polyclonal antibodies against an SH2/SH3/PH domain containing protein of the invention such as NEDD9 or PHLDA1, or a peptide of the invention, various host animals can be immunized by injection with the protein, including but not limited to rabbits, chickens, mice, rats, sheep, goats, etc. Further, the polypeptide or fragment or analogue thereof can be conjugated to an immunogenic carrier, e.g., bovine serum albumin (BSA) or keyhole limpet hemocyanin (KLH). Also, various adjuvants may be used to increase the immunological response, including but not limited to Freund's (complete and incomplete), mineral gels such as aluminium hydroxide, surface active substances such as rysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum.

Screening for the desired antibody can also be accomplished by a variety of techniques known in the art. Assays for immunospecific binding of antibodies may include, but are not limited to, radioimmunoassays, ELISAs (enzyme-linked immunosorbent assay), sandwich immunoassays, immunoradiometric assays, gel diffusion precipitation reactions, immunodiffusion assays, in situ immunoassays, Western blots, precipitation reactions, agglutination assays, complement fixation assays, immunofluorescence assays, protein A assays, and immunoelectrophoresis assays, and the like (see, for example, Ausubel et al., Current Protocols in Molecular Biology, Vol. 1, John Wiley & Sons, Inc., New York (1994)). Antibody binding may be detected by virtue of a detectable label on the primary antibody. Alternatively, the antibody may be detected by virtue of its binding with a secondary antibody or reagent which is appropriately labelled. A variety of methods for detecting binding in an immunoassay are known in the art and are included in the scope of the present invention.

The antibody (or fragment thereof) raised against an SH2/SH3/PH domain containing protein or peptide of the invention has binding affinity for that protein. Preferably, the antibody (or fragment thereof) has binding affinity or avidity greater than about 10⁵ M⁻¹, more preferably greater than about 10⁶ M⁻¹, more preferably still greater than about 10⁷ M⁻¹ and most preferably greater than about 10⁸ M⁻¹.

In terms of obtaining a suitable amount of an antibody according to the present invention, one may manufacture the antibody(s) using batch fermentation with serum free medium. After fermentation the antibody may be purified via a multistep procedure incorporating chromatography and viral inactivation/removal steps. For instance, the antibody may be first separated by Protein A affinity chromatography and then treated with solvent/detergent to inactivate any lipid enveloped viruses. Further purification, typically by anion and cation exchange chromatography may be used to remove residual proteins, solvents/detergents and nucleic acids. The purified antibody may be further purified and formulated into 0.9% saline using gel filtration columns. The formulated bulk preparation may then be sterilised and viral filtered and dispensed.

### Diagnosis of Predisposition to Hypersensitivity

In a further aspect, the invention relates to a method of diagnosing a predisposition to develop a hypersensitivity associated disease or disorder in a subject. The method comprises the steps of obtaining a nucleic acid sample from the subject and analysing the nucleic acid sample for the presence or absence of at least one mutation in a nucleic acid encoding an SH2/SH3/PH domain containing protein of the invention. Preferably, the SH2/SH3/PH domain containing protein is NEDD9 or PHLDA1.

Hypersensitivity associated diseases or disorders that may be diagnosed by the methods of the invention include, but are not limited to, anaphylaxis, drug reactions, skin allergy, eczema, allergic rhinitis, urticaria, atopic dermatitis, allergic contact allergy, food allergy, allergic conjunctivitis, insect venom allergy and respiratory diseases associated with airway inflammation.

The respiratory diseases associated with airway inflammation may include, but are not limited to, asthma, allergic asthma, intrinsic asthma, occupational asthma, acute respiratory distress syndrome (ARDS) and chronic obstructive pulmonary disease (COPD).

Nucleic acids for diagnosis may be obtained from the cells derived from various sources in a subject including but not limited to blood, urine, saliva, tissue biopsy and autopsy material. A variety of techniques have been used to identify sequence variations in nucleic acids. For example, Hi-Res Melting, a post-PCR technique for homogeneous mutation scanning and genotyping (Hi-Res LightScanner), Restriction Fragment Length Polymorphism (RFLP) analysis which detects restriction sites generated by mutations or alterations in nucleotide sequences (see Kan et al., Lancet 2(8096):910, (1978)), denaturing gradient gel electrophoresis and single stranded DNA electrophoretic mobility studies which identify nucleotide sequence differences through alterations in the mobility of bands in electrophoresis gels (see Myers et al., Nature 313:495, (1985); Orita et al., Proc. Natl. Acad. Sci. USA 86:2766, (1989)), chemical cleavage analysis which identifies mismatched sites in heteroduplex DNA (see Cotton, Proc. Natl. Acad. Sci. USA 85:4397, (1988)), and RNase cleavage analysis which identifies mismatched sites in RNA-DNA or RNA-RNA heteroduplexes (see Myers et al., Science 230:1242, (1985); Maniatis et al., U.S. Pat. No. 4,946,773).

Mutations may be detected at the level of DNA using a variety of techniques known in the art. Genomic DNA may be used directly for detection or may be amplified enzymatically using the polymerase chain reaction (PCR) (Saiki et al., Nature 324:163-166 (1986)) prior to analysis. RNA or cDNA may also be used for this purpose. As an example, PCR primers complementary to the nucleic acid encoding the SH2/SH3/PH domain containing protein of the invention can be used to identify and analyse mutations. For example, deletions and insertions may be detected by a difference in the size of the amplified product in comparison to that of a wild-type genotype. Point mutations may be identified by hybridizing amplified DNA to radiolabeled RNA encoding the SH2/SH3/PH domain containing protein or alternatively, radiolabeled antisense DNA sequences. Perfectly matched sequences can be distinguished from mismatched duplexes by RNase A digestion or by differences in melting temperatures.

Mutations may also detected by an alteration in electrophoretic mobility of nucleic acid fragments or proteins in gels with or without denaturing agents. Small sequence deletions and insertions can be visualized by high resolution gel electrophoresis. Nucleic acid fragments of different sequences may be distinguished on denaturing formamide gradient gels in which the mobilities of different fragments are retarded in the gel at different positions according to their specific melting or partial melting temperatures (for example see Myers et al., Science 230:1242 (1985)).

Sequence changes at specific locations may also be revealed by nuclease protection assays, such as RNase and S1 protection or the chemical cleavage method (for example see Cotton et al., Proc. Natl. Acad. Sci. (USA) 85:4397-4401 (1985)).

Thus, the detection of a specific DNA sequence may be achieved by methods such as hybridization, RNase protection, chemical cleavage, direct DNA sequencing or the use of restriction enzymes, (for example Restriction Fragment Length Polymorphisms (RFLP)) and Southern blotting of genomic DNA. In addition to more conventional gel-electrophoresis and DNA sequencing, mutations can also be detected by in situ analysis.

In accordance with the present invention, kits containing a means for obtaining a nucleic acid sample from a subject and a means for analysing the nucleic acid sample for the presence or absence of at least one mutation that modulates the activity of an SH2/SH3/PH domain containing protein of the invention may be prepared.

Such kits may be used, for example, for the diagnosis of a predisposition to developing an allergic disease or condition in a subject. Kits of the invention comprise is one or more means for obtaining a nucleic acid sample from the cells of a subject. Cells may be derived from various sources in a subject including but not limited to blood, urine, saliva, tissue biopsy and autopsy material. Additionally, kits of the invention comprise a means for analysing the nucleic acid sample for the presence or absence of at least one mutation that modulates the activity of an SH2/SH3/PH domain containing protein of the invention such as NEDD9 or PHLDA1. Examples of such mutations include, but are not limited to deletions, insertions, translocations, inversions, and base substitutions of one or more nucleotides.

Kits according to the invention may also include other components required to conduct the methods of the present invention, such as buffers and/or diluents. The kits typically include containers for housing the various components and instructions for using the kit components in the methods of the invention.

### Compositions and Routes of Administration

The present invention contemplates the use of compositions comprising an SH2/SH3/PH domain containing protein such as NEDD9 or PHLDA1, a peptide of the invention, mixtures of different SH2/SH3/PH domain containing proteins, mixtures of peptides, and combinations of SH2/SH3/PH domain containing proteins and peptides. The compositions may be included in a pharmaceutical composition comprising a pharmaceutically acceptable carrier, adjuvant and/or diluent. Additionally or alternatively, the compositions of the invention may comprise an immunosuppressive agent, for example an anti-inflammatory compound or a bronchodilatory compound.

In other embodiments, the immunosuppressive agents may be cyclosporines, tacrolimus, sirolimus, mycophenolate mofetil, methotrexate, chromoglycalates, theophylline, leukotriene antagonist, and antihistamine, or a combination thereof.

The immunosuppressive agent may also be an immunosuppressive drug or a specific antibody directed against B or T lymphocytes, or surface receptors that mediate their activation. For example, the immunosuppressive drug may be cyclosporine, tacrolimus, sirolimus, mycophenolate mofetil, methotrexate, chromoglycalates, theophylline, leukotriene antagonist, and antihistamine, or a combination thereof.

In addition, the pharmaceutical composition for use in accordance with the invention may still further comprise a steroid, such as a corticosteroid.

In another embodiment, the composition further comprises a steroid.

The invention also relates to a method for treating or preventing a hypersensitivity disease or disorder in a subject comprising the administration of an effective amount of the composition.

Further embodiments of the invention provide for the use of a protein comprising at least one of an Src homology 2 (SH2) domain, an Src homology 3 (SH3) domain, or a pleckstrin homology (PH) domain, for the manufacture of a medicament for the treatment of a hypersensitivity disease or disorder. Preferably, the protein is NEDD9 or a fragment or variant thereof. Preferably, the protein is PHLDA1 or a fragment or variant thereof.

Also described herein is the use of a peptide comprising an amino acid sequence corresponding to at least a portion of:

(i) an Src homology 3 (SH3) domain, or

(ii) a pleckstrin homology (PH) domain,

for the manufacture of a medicament for the treatment of a hypersensitivity disease or disorder. Preferably, the peptide comprises an amino acid sequence selected from the group consisting of SEQ ID NO: 7, SEQ ID NO: 8, SEQ ID NO: 10, SEQ ID NO: 11, SEQ ID NO: 12, and variants and fragments thereof.

In one embodiment, the peptide comprises an amino acid set forth in SEQ ID SEQ ID NOs: 14-273.

In another embodiment, the peptide comprises an amino acid set forth in any one of SEQ ID NOs: 274-488.

Compositions may be administered by standard routes. In general, the compositions may be administered by the parenteral (e.g., intravenous, intraspinal, subcutaneous or intramuscular). More preferably the compositions may be administered topically, orally, or intra nasally. Administration may be systemic, regional or local. The particular route of administration to be used at any given time will depend on a number of factors, including the nature of the condition to be treated, the severity and extent of the condition, the required dosage of the particular composition to be delivered and the potential side-effects of the composition.

The carriers, diluents and adjuvants must be “acceptable” in terms of being compatible with the other ingredients of the composition, and not deleterious to the recipient thereof. Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl stearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono-or di-oleate, -stearate or-laurate, polyoxyethylene sorbitan mono-or di-oleate, -stearate or-laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The topical formulations of the present invention, comprise an active ingredient together with one or more acceptable carriers, and optionally any other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving the active ingredient in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by: autoclaving or maintaining at 90° C.-100° C. for half an hour, or by filtration, followed by transfer to a container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those described above in relation to the preparation of drops. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil, wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes are generally derived from phospholipids or other lipid substances, and are formed by mono-or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

### Dosages

Suitable compositions for use in accordance with the methods of the invention of the invention may be administered as compositions either therapeutically or preventively. In a therapeutic application, compositions are administered to a patient already suffering from a disease or condition, in an amount sufficient to cure or at least partially arrest the disease or condition and its complications. The composition should provide a quantity of the agent sufficient to effectively treat the patient.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours ; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Typically, in therapeutic applications, the treatment would be for the duration of the disease state or condition. Further, it will be apparent to one of ordinary skill in the art that the optimal quantity and spacing of individual dosages will be determined by the nature and extent of the disease state or condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

The invention will now be described with reference to specific examples, which should not be construed as in any way limiting the scope of the invention.

### Examples

### Example 1

### Identification of Putative SH2/SH3/PH Domain Containing Proteins Involved in Mast Cell Activation

A computer based approach was used to identify novel genes in activated mast cells that encode for intracellular signaling products. This was achieved by screening gene expression data obtained from resting and activated bone-marrow derived human mast cells for genes encoding SH2 and/or SH3 and/or PH domains.

Bone marrow derived mast cells (BMMC) were generated as described in Liu et al. (J. Allergy Clin Immunol 118:496-503, (2006)), mRNA was isolated from resting and IgE cross-link-stimulated BMMC and gene expression arrays were performed following the Affymetrix standard protocol explained in Lui et al. (J Allergy Clin Immunol 118:496-503, (2006)).

Domain sequence searches were performed using a LINUX based platform for gene sequence scanning to identify SH2/SH3/PH domain containing proteins expressed by BMMC. Of the nearly 15000 Affymetrix probes found to be expressed in mast cells, 207 genes encode proteins that have SH2, SH3 and/or PH domains and 23 (11%) of them were found to be either upregulated or downregulated upon FcεRI cross linking.

A number of genes encoding molecules previously unidentified as members of the mast cell IgE signaling pathway were considered as possible players in mast cell activation process, based on their reported gene sequence characteristics and biological function in other cells types. Among them, NEDD9 (neural precursor cell-expressed developmentally downregulated gene 9), and PHLDA1 were selected for further study on the basis of their structural characteristics and biological activity.

### Example 2

### Role of NEDD9and PHLDA1 in Mast Cell Activation

### (i) NEDD9 a Highly Selective Docking Protein Involved in Signal Transduction

The human NEDD9 gene (neural precursor cell-expressed developmentally downregulated gene 9) (GenBank, IDs: NM_{—}006403.2 and NM_{—}182966.2, Ensembl ID: ENSG00000111859, Entrez gene ID 4739 and Uniprot ID Q14511), also known as CasL (Crk-associated substrate lymphocyte type) and HEF1 (human enhancer of filamentation 1) encodes the expression of an unprocessed precursor protein of 834 amino acids. NEDD9 contains an SH3 domain and a domain rich in SH2-binding sites.

NEDD9 isoform 1 has the UniProt identifier Q14511 and is identical to isoform CRA_b (UniProt identifier Q5T9R4). NEDD9 isoform 1 has a SH3 domain at position 10-64, a CC motif at position 633-656, three phosphorylation sites at positions 92, 166, 177 and 189 and a cleavage site at position 363.

Isoform CRA_a (UniProt identifier Q5T9R4) is also an 834 amino acid protein that differs from isoform 1 at sequence positions 2, 3 and 4. It belongs to the Cas family and, like all of the family members, features an N-terminal SH3 domain comprising 60 amino acids that correspond to residues 7 to 66 in the sequence defined by REFSEQ accession NM_{—}006403.2, which is isoform 1 (Q14511). It also contains a central domain incorporating multiple potential SH2-binding sites and a C-terminal domain that has a divergent helix-loop-helix (HLH) motif. The SH2-binding sites putatively bind CRK, NCK and ABL SH2 domains. The HLH motif confers specific interaction with the HLH proteins, ID2, E12 and E47. Post-translational modifications that arise from cell cycle-regulated processing produces four proteins from isoform 1: p115, p105, p65, and p55. Protein isoform 1 p115 arises from isoform 1 p105 phosphorylation and appears later in the cell cycle. Isoform 1 p55 arises from isoform 1 p105 as a result of cleavage at a caspase cleavage-related site and it appears specifically at mitosis.

Splice variance in the transcripts of the human NEDD9 gene may give rise to protein isoform 2 (UniProt: Q5XKI0), isoform CRA_c (REFSEQ: EAW55302.1) and isoform CRA_d (REFSEQ: EAW55303.1). The HEF1 protein isoform CRA_c comprises 800 amino acids and is similar to isoforms 1 and CRA_a, differing by loss of a small number of potential ligand motifs in the central SH2-motif rich domain. The transcript for CRA_d encodes a protein of 688 amino acids that lacks the N-terminal SH3 domain. Isoform 2 by contrast is only a 174 amino acid protein that is identical to the N-terminal residues 1 through 154 of HEF1 isoform 1 and is essentially only the SH3 domain and a drastically reduced SH2-ligand domain. Additional human NEDD9-encoded protein variants are listed in the UniProt database including a protein (UniProt: Q5TI59) truncated after residue Pro-148, which is essentially a slightly smaller version of isoform 2.

With the exception of the CRA_d isoform, the N-terminal SH3 domain is present in each of the NEDD9 isoforms referred to above.

NEDD9 is a multifunctional docking protein involved in propagating intracellular signals and is expressed in a wide variety of tissues in the nucleus, cytoplasm and structures such as Golgi and lamellipodia. The SH3 domain (aa 2-64) and coiled-coil domain (aa 633-656) of NEDD9 confer to the potential to interact with high selectivity with Lck, Lyn and Fyn, three signal transduction molecules downstream of cell surface receptors. Other protein interaction motifs are a Serine-rich domain (aa 400-558) and a conserved C-terminal domain containing a helix-loop-helix (HLH) motif (aa 710-760). The SH3 fold of NEDD9 consists of two anti-parallel beta sheets that lie at right angles to each other. Within the fold, there are two variable loops, referred to as RT and n-Src loops. When SH3 binds to its ligand, the proline rich ligand adopts a poly-L-proline type II helix conformation, with the PPII helical structure recognised by a pair of grooves on the surface of the SH3 domain that bind turns of the helix. The SH3 grooves are formed by a series of nearly parallel, well-conserved aromatic residues.

NEDD9 has been identified to interact with several intracellular proteins involved in cell signalling. Table 1 describes the interacting protein, type of interaction and experimental method used for identification.

TABLE 1

| Name of Interactor | Experiment Type | Type |
|---|---|---|
| ABL | In Vivo; In Vitro | Direct |
| Breast cancer anti estrogen | In Vivo | Direct |
| resistance 3 | | |
| CRK | In Vivo; In Vitro | Direct |
| CRK associated substrate | In Vivo; Yeast 2 Hybrid | Direct |
| CRKL | In Vivo; In Vitro | Direct |
| Cadherin 1 | In Vitro | Direct |
| CasL interacting molecule | In Vivo; In Vitro | Direct |
| Choline acetyltransferase | In Vivo | Direct |
| FAK | In Vivo; In Vitro; Yeast 2 Hybrid | Direct |
| Inhibitor of DNA binding 2 | In Vivo; Yeast 2 Hybrid | Direct |
| Itchy homolog E3 ubiquitin | In Vivo; In Vitro; Yeast 2 Hybrid | Direct |
| protein ligase | | |
| Lck | In Vitro | Direct |
| Lyn | In Vivo | Direct |
| NCK1 | In Vivo; In Vitro | Direct |
| PTK2B protein tyrosine | In Vivo | Direct |
| kinase 2 beta | | |
| Paxillin | In Vivo | Direct |
| Protein tyrosine | In Vitro | Direct |
| phosphatase | | |
| nonreceptor type 12 | | |
| SH2D3C | In Vivo; In Vitro | Direct |
| SHP2 | In Vivo; In Vitro | Direct |
| SMAD, mothers against | In Vivo | Direct |
| DPP homolog 2 | | |
| (Drosophila) | | |
| SMAD3 | In Vivo; In Vitro; Yeast 2 Hybrid | Direct |
| Sma and Mad related | In Vivo; Yeast 2 Hybrid | Direct |
| protein 1 | | |
| Thyroid hormone | Yeast 2 Hybrid | Direct |
| receptor interactor 6 | | |
| Transcription factor 3 | Yeast 2 Hybrid | Direct |
| Zyxin | In Vivo; In Vitro; Yeast 2 Hybrid | Direct |
| Enhancer of | In Vivo; In Vitro; Yeast 2 Hybrid | Direct |
| filamentation 1 | | |
| Epididymal sperm | Yeast 2 Hybrid | Direct |
| binding protein 1 | | |
| HSA9761 | Yeast 2 Hybrid | Direct |
| Fyn | In Vivo; In Vitro | Direct |
| Guanine nucleotide | In Vivo; In Vitro | Direct |
| releasing factor 2 | | |

### (ii) PHLDA1 a Molecule Involved in Intracellular Signaling Downstream Receptor Binding

The human PHLDA1 gene (Entrez Gene ID 22822; Pleckstrin homology-like domain family A member 1) also known as TDAG51 (T cell death associated gene 51), encodes the expression of a transcript of 5913 nucleotides, NCBI REFSEQ accession NM_{—}007350, and cDNA sequences have been reported that may be translated into proteins of 401 (REFSEQ: NP_{—}031376) or 400 (REFSEQ: EAW97312.1) amino acids. The preferred name for the protein encoded by the PHLDA1 gene is Pleckstrin homology-like domain family A member 1 (PHLDA1) and has the UniProt identifier Q8WV24. PHLDA1 is expressed in a wide variety of tissues in the cytoplasm, cytoplasmic vesicle membrane, nucleus and nucleolus, and features an N-terminal pleckstrin homology, or PH, domain comprising 133 amino acids that correspond to residues 9 to 141 in the sequence defined by REFSEQ accession NP_{—}031376. PHLDA1 also incorporates a 15 (prolylglutaminyl-) repeat sequence immediately followed by a 14 (prolylhistidyl-) repeat sequence toward the C-terminus. The PH domain itself includes either a 15 (NP_{—}031376) or 14 (EAW97312.1) residue poly(glutamine) element.

There are no post-translational modifications to the protein listed in the UniProt Knowledgebase. The UniProt Knowledgebase describes only a 259 amino acid protein (Q8WV24) circumscribed as the variant, PHLDA1 CRA_a (e.g. REFSEQ: AAH18929.3, AAI10821.1 and AI26426.2) in the Entrez database. This protein lacks a 141 residue sequence from the N-terminus of the predicted longer forms and differs from NP_{—}031376 by having a single glutamine residue deletion at the beginning of the poly(glutamine) repeat sequence in the PH domain, which is 14 residues in length in this form. The protein name PHLDA1 commonly refers to this 259 residue shorter form. A cDNA sequence (REFSEQ: AAI30428) is predicted to encode a protein of 312 residues with the PH domain being encompassed by residues 62 to 194.

The PH domain is present in all of the isoforms described above and typically comprises about 100 amino acids. PH domains have a common structure consisting of two perpendicular anti-parallel beta sheets, followed by a C-terminal amphipathic helix. The loops connecting the beta-strands differ greatly in length, making the PH domain relatively difficult to detect. There are three members of the pleckstrin homology-like domain family A, PHLDA1 to PHLDA 3, each of which differs significantly in the amino acid sequence of the constituent PH domain. PHLDA1 differs from the other A family members in that it incorporates an additional 43 amino acid insertion into the loop that includes the poly(glutamine) element.

Table 2 below lists some of the molecules known to interact with the PHLDA1 gene product PHLDA1, along with and type of interaction and experimental method used for identification.

TABLE 2

| Name of Interactor | Experiment Type | Type |
|---|---|---|
| EIF3S7 | In Vivo; In Vitro; Yeast 2 Hybrid | Direct |
| Keratin associated | Yeast 2 Hybrid | Direct |
| protein 4-12 | | |
| MyoD family inhibitor | Yeast 2 Hybrid | Direct |
| Phospholipid scramblase 1 | Yeast 2 Hybrid | Direct |
| Poly(A) binding protein 4 | In Vivo; In Vitro; Yeast 2 Hybrid | Direct |
| Ribosomal protein L14 | In Vivo; In Vitro; Yeast 2 Hybrid | Direct |

### Example 3

### Expression Kinetics of NEDD9 after IgE Stimulation in Human and Mouse Mast Cells

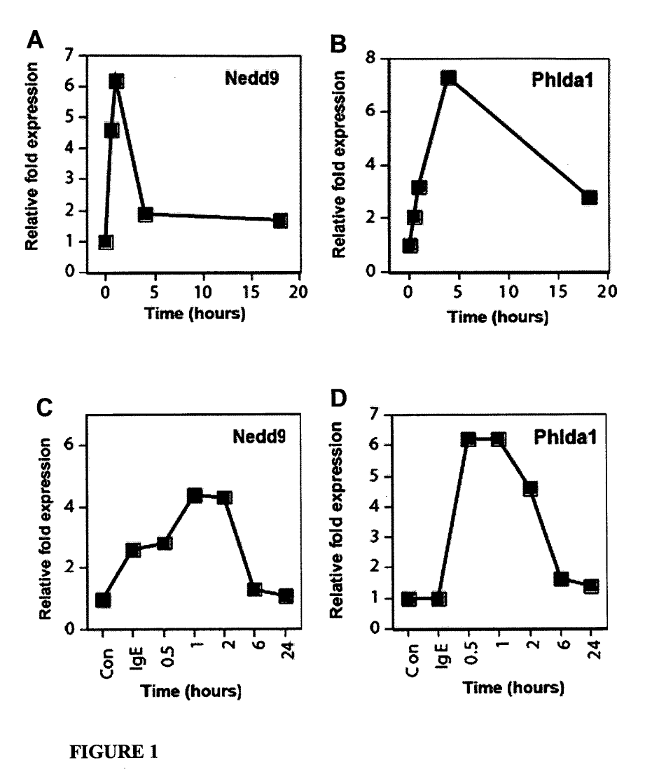

The expression kinetics of NEDD9 was assessed after IgE stimulation of human and mouse mast cells (derived from human or mouse bone marrow stem cells). Cells were incubated with anti DNP/IgE mab (10-500 ng/ml) for 2 hours and FcεRI activation by crosslink with a DNP/albumin solution (100 ng/ml) for the specified length of time (0 to 4 hours). Gene expression was measured by quantitative real-time PCR using NEDD9 specific primers for both species and analysis was performed using relative quantification comparative Ct method (see Livak and Schmittgen, Methods 25:402-408, (2001)). NEDD9 was upregulated early after human mast cell IgE crosslinking, peaking at 2.5 hours and decreasing 5 hours later to reach a steady expression just above baseline expression (FIG. 1A). NEDD9 was upregulated early after human mast cell IgE crosslinking, peaking at 2.5 hours and decreasing 5 hours later to reach a steady expression just above baseline expression (FIG. 1A). Expression levels of PHLDA1 were also found upregulated in response to IgE crosslinking in human mast cells, reaching the peak 5 hours after IgE stimulation (FIG. 1B). Similarly, an early upregulation of NEDD9 and PHLDA1 expression was observed soon after IgE stimulation of mouse mast cells reaching the peak at 1 hour for NEDD9 and 30 minutes for PHLDA1 after IgE stimulation and decreasing, in both cases, to baseline by 6 hours (FIGS. 1C and 1D).

### Example 4

### NEDD9 and PHLDA1 Expression Kinetics in a RBL-2H3 Cells in Response to FcεRI Crosslinking and Ionomycin Stimulation

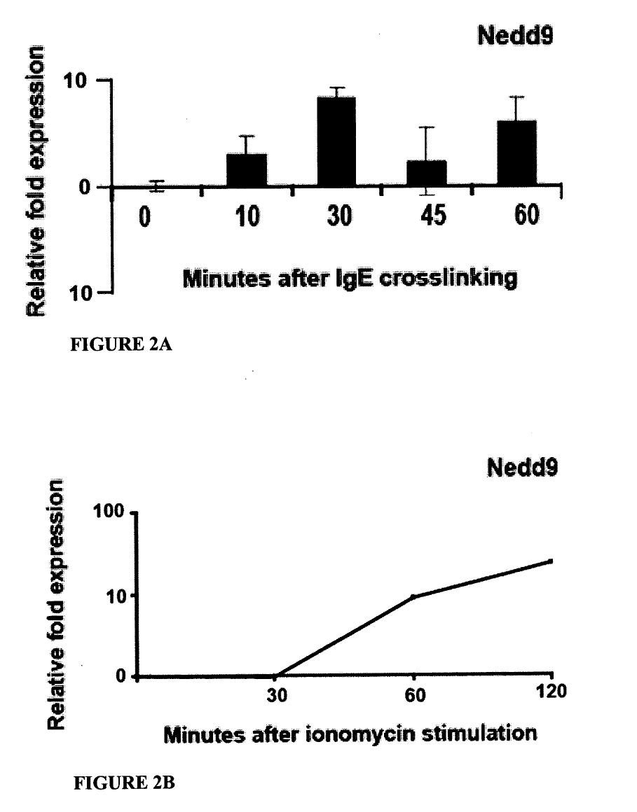

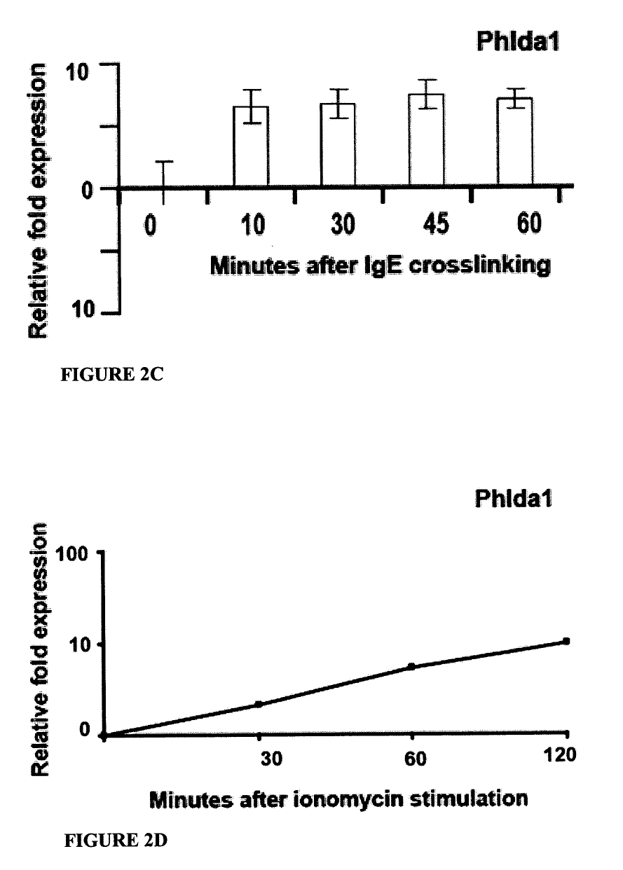

The expression kinetics of NEDD9 and PHLDA1 was assessed in RBL-2H3 cells, a rat mast cell line expressing FcεRI. Gene expression was measured by SYBR-Green-based quantitative real-time PCR using specific primers for each gene. Each experimental sample was assayed using three replicates for each primer, including the β-actin specific primer that was used as an internal standard. Negative controls lacking the cDNA template were run with every assay to assess specificity. Primer Express software (Applied Biosystems) was used for primer design. A threshold cycle (Ct) was determined for each sample. PCR assays showing non-specific products at the end point were excluded from further data analysis. Relative quantification using comparative Ct method was used for analysing results (see Livak and Schmittgen, Methods 25:402-408, (2001)). The expression of NEDD9 and PHLDA1 was significantly upregulated in response to cell activation via FcεRI crosslinking (FIGS. 2A and 2C). Briefly, 5×10⁶ RBL-2H3 cells were incubated with anti DNP/IgE mab (100 ng/ml) for 2 hours in F15 tissue culture medium supplemented with 5% foetal calf serum, L-glutamine, penicillin/streptomycin. The FcεRI was then crosslinked with DNP/albumin solution (100 ng/ml) for the specified length of time. Analysis of the expression kinetics following FcεRI crosslinking showed both genes act early after stimulation suggesting its involvement in modulating the cascade of events leading to mast cell activation.

Although the most specific mast cell activation pathway involves the aggregation of FcεRI bound IgE on the cellular surfaces, mast cells can also be activated by various means, including FcγR, Toll-like receptors (TLRs) and other IgE independent triggers. Accordingly, it was assessed whether NEDD9 and PHLDA1 are involved in other intracellular signaling cascades apart from the one resulting from FcεRI crosslinking. Ionomycin is an ionophore that translocates calcium from the extracellular the intracellular space, leading to the rise in intracellular free calcium, and the subsequent stimulation of mast cell effector functions, including the release of the content in preformed granules. Mast cells were incubated with 1 μM ionomycin and the expression levels of NEDD9 and PHLDA1 were analysed at different time points by QRT-PCR. In both cases, gene expression levels were found upregulated in response to ionomycin at all time points analysed (30, 60 and 120 min) (FIGS. 2B and 2D). Similar to activation via FcεRI crosslinking, analysis of expression kinetics in response to ionomycin showed NEDD9 and PHLDA1 appear to act early after stimulation again supporting their involvement in modulating the cascade of events leading to mast cell activation.

### Example 5

### Increased Mast Cell Degranulation Following Silencing of NEDD9 Expression In-Vitro and In-Vivo

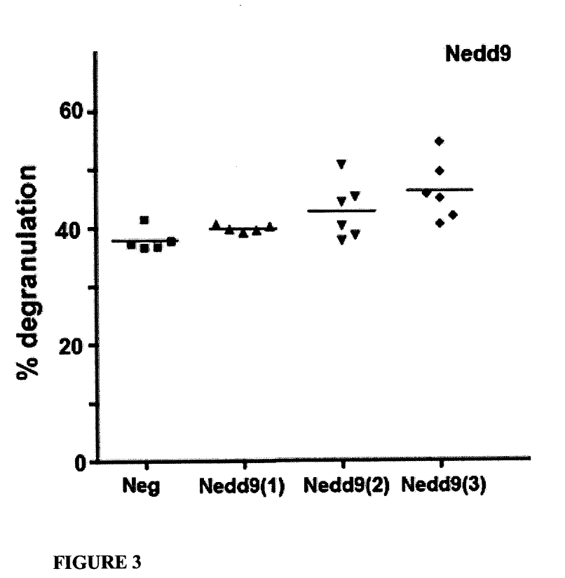

A mast cell degranulation assay was used to assess whether silencing of NEDD9 expression could influence the effector functions of mast cells in vitro. RBL-2H3 rat mast cells were incubated with DNP-IgE for 30 minutes in the presence of 3 different siRNAs targeting NEDD9 gene expression which were specifically designed to interfere with NEDD9 expression. siRNA were purchased from AMBION Silencer Validated siRNAs and transfection was performed using the recommended protocol (www.ambion.com). An additional siRNA not targeted to any gene was used as a control. After overnight incubation with IgE/DNP, the FcεRI was activated via crosslinking with DNP/albumin conjugate. Mast cell degranulation was then calculated by β-hexosaminidase release and used as an indicator of mast cell effector function in response to FcεRI crosslinking. As shown in FIG. 3, suppression of NEDD9 protein expression by siRNA resulted in an increase in the percentage of mast cell degranulation compared to siRNA negative controls.

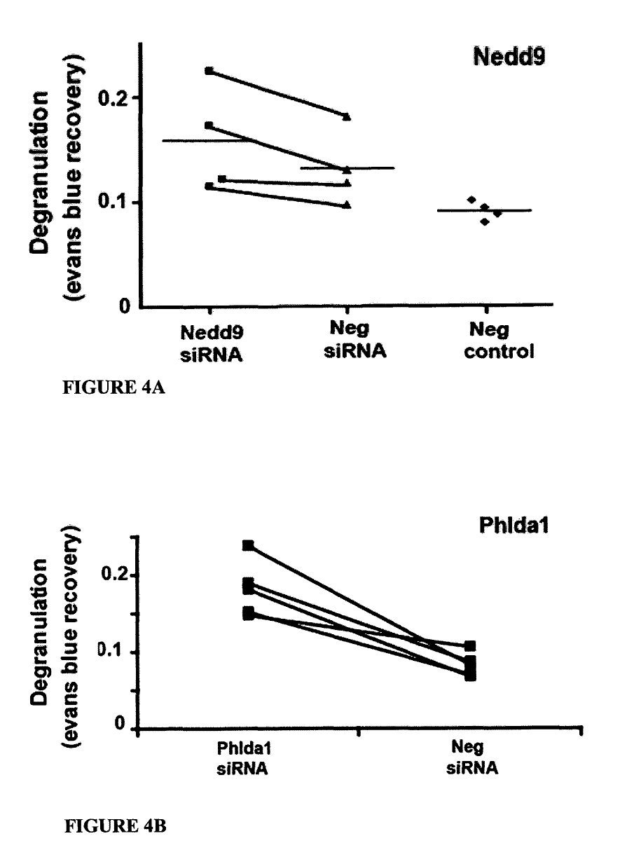

The effect of decreasing NEDD9 and PHLDA1 expression was then assessed in-vivo using the passive cutaneous anaphylaxis (PCA) reaction. Lewis rats were injected subcutaneously in the base of the ears with a combination of NEDD9 siRNAs or PHLDA1 siRNAs (silencer validated siRNAs from AMBION) and IgE-DNP. Control siRNA/DNP-IgE was injected in the left ear. FcεRI crosslinking was induced by challenging intravenously with DNP/albumin 24 hours later. Ear mast cell degranulation was measured 30 minutes later by Evan's blue recovery. Local administration of NEDD9 or PHLDA1 siRNA to the right ear resulted in increased degranulation compared to the left ear treated with siRNA control (FIGS. 4A and 4B).

### Example 6

### Peptides Targeting the SH3 Domain of NEDD9 Neutralise Mast Cell Activation In-Vitro and In-Vivo

### Materials and Methods

(i) Peptide Design

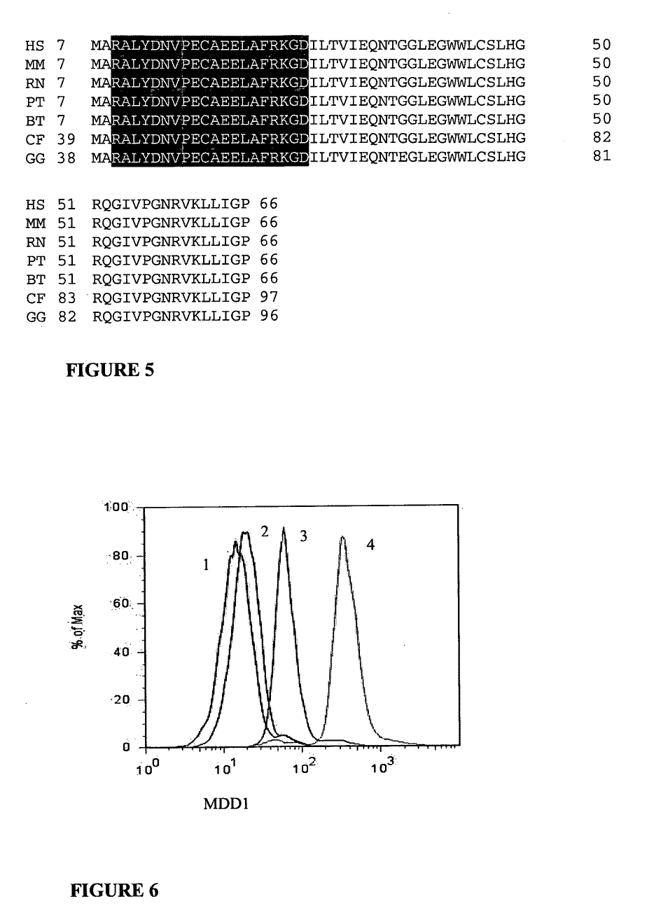

SH3 domains recognise proline rich linear motifs involved in assembling intracellular signalling complexes and regulatory processes in signal transduction and cell activation. The SH3 domain of NEDD9 is conserved across species from humans to flies (Homologene NCBI and FIG. 5) suggesting its importance in signal transduction activity. The sequence of the N-terminal SH3 domain corresponds to residues 7 to 66 in the sequence defined by REFSEQ accession NM_{—}006403.2 (HEF1 isoform 1 UniProt: Q14511).

Residues 7-66 of HEF1 isoform1 (UniProt: Q14511.1), the N-terminal SH3 domain encoded by human NEDD9 (Entrez Gene ID 4739)

(SEQ ID NO: 7)

MARALYDNVP₁₀ ECAEELAFRK₂₀ GDILTVIEQN₃₀
TGGLEGWWLC₄₀ SLHGRQGIVP₅₀ GNRVKLLIGP₆₀

NEDD9 competitor peptides were constructed to specifically interrupt the binding of the NEDD9 SH3 domain with target ligands. Peptides were synthesised chemically at the Australian National University Australian Cancer Research Foundation Biomolecular Resource Facility. Solid-phase synthesis on Rink resin, 4-(2′,4′-dimethoxyphenyl-Fmoc-aminoethyl)-phenoxy polystyrene, was performed on a Symphony Peptide Synthesiser (Rainin Instrument Company Oakland Calif.) using standard Fmoc (9-fluorenylmethyloxycarbonyl) protocols and purified by reverse-phase HPLC. The identity of the purified products was confirmed by mass spectrometry.

A 20-mer peptide (MDD1) was constructed corresponding to residues 3-22 of the human sequence (HS) of the NEDD9 SH3 domain depicted in FIG. 5. The peptide MDD1 was chosen first for activity testing because it includes the cluster of negatively charged residues in the RT (variable) loop of the SH3 domain. The MDD1 peptide is specific for NEDD9 as confirmed by peptide blast against all species. The sequence of MDD1 is conserved in mammals including homo sapiens, mus musculus, rattus norvegicus, pan troglodytes, bos taurus, canis familiaris as well as gallus gallus (FIG. 5—see highlighted sequence).

A peptide containing the same amino acids in a scrambled sequence (MDD1_{scram}) was also synthesised to determine the necessity for preserving the native sequence. They were synthesised in the form of N-acetyl α-carboxamides to both suppress terminal ionisation and degradation by exopeptidases. Biotynalated forms were also produced. The peptides sequences are detailed below:

| | | |
|---|---|---|
| MDD1 peptide: | RALYDNVPECAEELAFRKGD | (SEQ ID NO: 8) |
| MDD1_{scram} | EALPGEDCAFRKDANRLVEY | (SEQ ID NO: 9) |

Two further analogues, MDD1S10 and MDD1A10 both lacking a cysteine residue at position 10, were also synthesised:

### (ii) MDD1 Uptake by RBL-2H3

RBL-2H3 cells were grown in F15 medium supplemented with 10% of fetal calf serum (FCS) and antibiotics. Cells were carefully detached from the culture flask with a cell scraper and plated at a concentration of 5×10⁵ cells/well in a 96 well microtiter plate. Cells were incubated with various concentrations of biotinylated MDD1 (0.1, 0.01 and 0.001 mM) overnight. Biotinylated MDD1 uptake by live cells was detected using streptavidin-APC in single-cell suspensions previously treated with a combined cytofix/cytopenn fixation and penneabilization solution kit following manufacture's instructions (Becton Dickinson, BD). Cells were then analysed using a BD FACscan flow cytometer.

(iii) RBL-2H3 Degranulation Assay

Mast cell degranulation was measured using the β-hexosaminidase release method. RBL-2H3 cells were grown in F15 medium supplemented with 10% of fetal calf serum (FCS) and antibiotics. Cells were carefully detached from the culture flask with a cell scraper and plated at a concentration of 5×10⁵ cells/well in a 96 well microtiter plate and incubated in the presence of MDD1 or SCR peptide overnight. RBL-2H3 cells were then incubated with 500 ng/ml anti-DNP IgE mb (SIGMA) for 2 hours at 37° C. After washing, cells were stimulated with 100 ng/ml DNP/albumin for 30 minutes. For non IgE specific stimulation 1 nM Ionomycin only was added to the cultures for 30 minutes. Supernatants were then collected and cell lysates were prepared using 0.1% Triton X-100. Lysate and supernatant samples (10 μl) were transferred into 96-well plates and 50 μl mM 1 mM p-nitrophenyl-N-acetyl-β-D-glucopyranoside in 50 mM citrate buffer was added to each well and incubated for 1 hour at 37° C. in the dark. The reaction was stopped by adding 100 μl 0.1M NaHCO₃/0.1M Na₂CO₃ into each well and the absorbance measured at 405 nm. Percentage degranulation was calculated by the following formula: OD supernatant/(OD supernatant+OD lysate)×100.

(iii) OVA-Induced Asthma Model

C57BL/6 mice were sensitised with 10 ug ovalbumin antigen (OVA) in saline intraperitoneally seven times given on alternate days. 40 days after first sensitisation dose, mice were challenged with intranasal OVA (20 ul in each nostril of a solution of 5 mg/ml of OVA in saline). Mice received two additional challenges of intranasal OVA at days 3 and 6 after the first challenge with OVA. Control mice were sensitised with OVA, but challenged intranasally with saline. Groups of mice receiving parenteral MDD1 treatment were intravenously injected with 8 mg/Kg of MDD1 three times, 6 hours prior to each OVA challenge. Nebulised MDD1 or MDD_{scram} (SCR) peptide was given 6 hours prior to each OVA challenge by nebulising 5 mice per cage with 8 ml of 1 mg/ml MDD1 or control peptide.

Functional lung studies and organ collection was performed one day after the final OVA challenge. Lung resistance was measured in anesthetized mice after increasing doses of β-methacholine. Tracheas were surgically exposed, cannulated, and connected to a rodent ventilator (flexivent; Scireq). Mice were allowed to stabilize and challenged with a saline aerosol followed by increasing concentrations of methacholine. Lung resistance was measured by plethysmography, using an apparatus and software supplied by Buxco. Lung resistance values are expressed as an average of each group of mice for each methacholine dose. For each mouse, individual values were calculated by averaging collected data (every 5 seconds during 5 minutes) for each methacholine dose.

### Immunostaining, Cytokine Expression

Blood smears were prepared and the percentage of eosinophils was calculated by morphological criteria after May-Grünwald Giemsa-staining. Bronchoalveolar lavage (BAL) was recovered by lavaging airways twice with 1 ml HANKS solution via the tracheal cannula while gently massaging the thorax. Lung lower left lobe was collected and homogenised. Cells were filtered through a cell strainer and cell suspensions prepared in staining medium (PBS with 1% FCS). BAL and lung samples were incubated with anti-CD11b, anti-CCR3, anti-CD4 and antiB220 mab markers for flow cytometric analysis using a FACScan equipped with CellQuest software.

Lung upper right lobe was collected and homogenised on ice. Total RNA was prepared using Trizol (GIBCO, Invitrogen). Purity of the RNA was determined by A260/A280. 5 μg RNA for each sample was reverse transcribed into cDNA using Omniscript RT kit (Qiagen). Cytokine specific predesigned TaqMan gene expression assay (Applied Biosystems) were used to measure cytokine relative gene expression following the manufacturer's instructions (Applied Biosystems). Each experimental sample was assayed in three replicates. β-actin specific primers were used as an internal standard. Negative controls with blank cDNA template were run with every assay to assess specificity. The cycling conditions were as follows: one cycle at 95° C. for 10 minutes, followed by 40 cycles of PCR amplification, each consisting of 95° C. for 15 seconds and 60° C. for 45 seconds. Sequence Detection Software (SDS v 1.2.2, Applied Biosystems) was used for analysis of the results. A threshold cycle (Ct) was determined for each sample. PCR assays showing non-specific products at the end point were excluded from further data analysis. Relative quantification using comparative Ct method was used for analysing results. The results were expressed as relative fold change over the values for non-allergic mice.

### Serum Ig Levels

OVA-specific IgE detection: 96-well maxisorp plates were coated overnight at 4° C. with anti-mouse IgE. 2% skim milk was used for unspecific binding blockade. Prediluted serum samples were incubated overnight, plates were loaded with OVA-biotin for 1.5 hours and a colometric reaction was developed using ABTS following manufacture's instructions. For OVA-specific IgG, 96-well maxisorp microtiter plates were pretreated with 0.2% glutaraldehyde and were coated with 10 μg/ml of OVA in 10 mM carbonate buffer (pH 9.6) for 3 hours at 37° C. Wells were blocked with 2% BSA in 10 mM carbonate buffer (pH 9.6) overnight at 4° C. in a humidified chamber. Serum samples were incubated at 37° C. for 1 hour. Peroxidase-conjugated anti-rat IgG followed by ABTS was used to develop the colorimetric reaction. Optical density was read in a microplate reader using a 405 nm filter. Levels of IgE and IgG were expressed as OD units.

### Passive Cutaneous Anaphylaxis Assay (PCA) in Mice

Left ears were treated with DMSO and right ears with 0.2% MDD1 or 0.1% dexamethasone in DMSO twice daily for four days. Mice were then sensitised with anti DNP-20 ul IgE antibody administered to the ears by intradermal injection using an insulin syringe. Local mast cell activation was measured by dye recovery in response to FcεRI crosslink 24 hours post-injection: 100 μg DNP-albumin/1% Evans blue in PBS was injected intravenously and ear biopsies taken after 30 minutes were incubated at 80° C. in 1 ml formamide for 2 hours. The absorbance supernatant was measured at 620 nm.

### Results

### (i) MDD1 Peptide is Biologically Active In-Vitro

MDD1 peptide was found to penetrate the cellular membrane. MDD1 peptide was directly conjugated to biotin and administered to RBL-2H3 mast cells. MDD1 peptide uptake was detected by intracellular FACS staining using streptavidin APC as a fluorochrome. All cells were positive for MDD1 peptide and the levels were observed to increase with peptide concentration (FIG. 6).

### (ii) MDD1 Peptide Inhibits Degranulation in RBL-2H3 Cells Following IgE Receptor Crosslinking

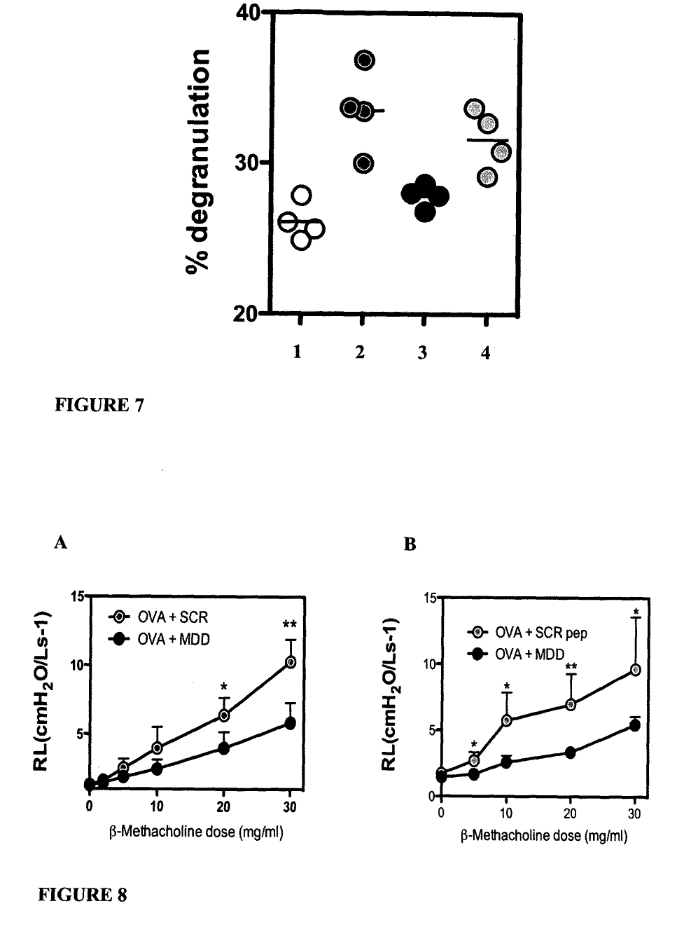

MDD1 peptide (SEQ ID NO: 8) was observed to inhibit mast cell activation after IgE receptor crosslinking as well as after non-specific stimulation (data not shown). RBL-2H3 cells not pre-treated with MDD1 peptide degranulated after IgE receptor crosslinking (FIG. 7). However, pretreatment of RBL-2H3 cells with MDD1 peptide was observed to inhibit degranulation upon IgE receptor crosslinking (FIG. 7). Pretreatment of RBL-2H3 cells with a scrambled peptide MDD1_{scram} (SEQ ID NO: 9) had only a minor inhibitory effect on degranulation following IgE receptor crosslinking (FIG. 7).

(iii) MDD1 Peptide Improves Lung Function in OVA-Sensitised Mice Following Challenge with β-Methacholine

The efficacy of MDD1 peptide in controlling asthmatic responses was tested in a mouse model of asthma driven by mast cell activation. Lung inflammation and asthmatic responses in this model are dependent on mast cell activation since mice lacking mast cells do not develop the disease.

OVA-sensitised mice treated with MDD1 at the time of antigen exposure had improved lung function after challenge with β-methacholine compared to mice treated with control peptide (FIG. 8). Lung resistance in OVA-sensitized mice treated with either MDD1 peptide or control (SRC) peptide was measured following increasing doses of β-methacholine. Lung resistance (RL) was reduced in mice treated with MDD1 peptide compared to mice treated with the control peptide. This effect was observed when peptides were administered intravenously (FIG. 8A), or administered locally to the lungs using a nebulised intranasal route (FIG. 8B).

(iv) MDD1 Peptide Reduces Inflammatory Responses During Asthmatic Reaction in OVA-Sensitized Mice Following Challenge with β-Methacholine

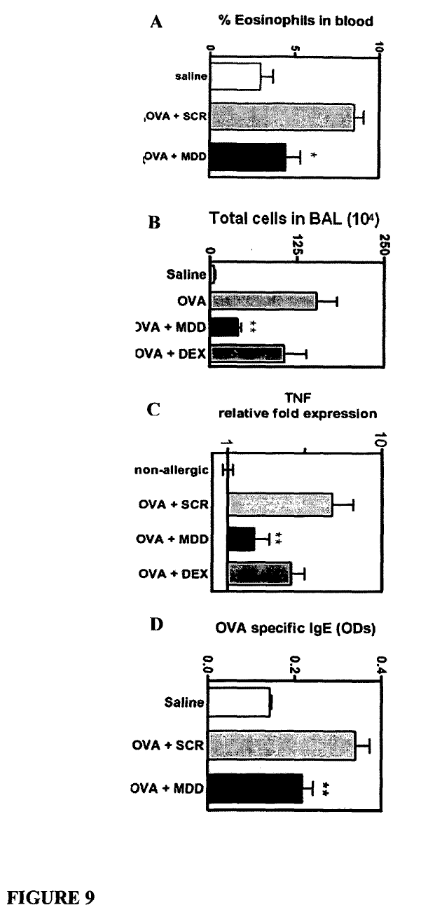

MDD1 peptide reduced the overall inflammatory response in the asthmatic reaction as demonstrated by decreased eosinophilia in blood (FIG. 9A), cellularity in broncho-alveolar lavages (FIG. 9B), cytokine production in inflammatory foci (FIG. 9C) and levels of antigen-specific IgE in serum of allergic mice compared to control treated mice (FIG. 9D). MDD1 peptide outperformed dexamethasone treatment (2 mg/kg i.p.) in some of the parameters tested (FIG. 9).

### (v) Topical Administration of MDD1 Peptide Reduces Allergic Responses

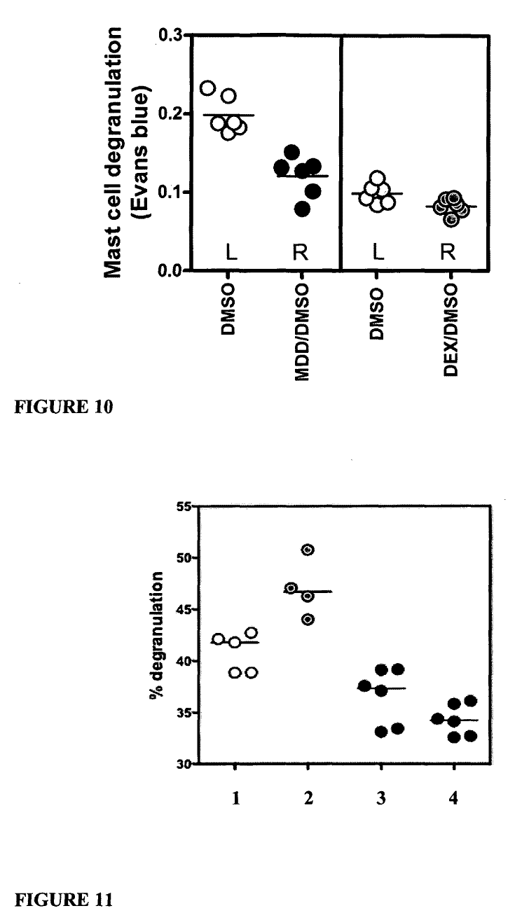

Atopic dermatitis and other allergic skin conditions are thought to be driven by mast cell responses. Topical administration of MDD1 peptide was demonstrated to stabilise skin mast cells. A 0.2% MDD1 peptide/DMSO preparation was topically applied to one ear of each mouse prior to IgE local sensitisation with anti DNP IgE antibody. Mast cells in biopsies from ears treated with MDD1 peptide/DMSO were found to have reduced levels of activation following IgE receptor crosslink, as determined by dye extravasation compared to contralateral ears treated with DMSO only (FIG. 10). MDD1 peptide acted locally with minimal systemic effects while local dexamethasone treatment resulted in systemic immunosuppresion.

### Example 7

### Peptide Competitors of the PH Domain of the PHLDA1 Gene Product Neutralise Mast Cell Activation

### Materials and Methods

### (i) Peptide Design

The PH domain of PHLDA1 comprises 133 amino acids and ranging from amino acid residues 9 to 141 in the sequence defined by REFSEQ accession NM_{—}006403.2. The sequence is highly conserved in mammalia. The PHLDA1 PH domain includes a large loop insertion as shown below (see bold italicised sequence: residues 37 to 79).

Residues 9-141 of PHLDA1 (UniProt: Q8WV24.1), the PH domain encoded by human PHLDA1 (Entrez Gene ID 22822)

(SEQ ID NO: 10)

ALKEGVLEKR₁₀ SDGLLQLWKK₂₀ KCCILTEEGL₃₀
LLIPPKQLQH₄₀ QQQQQQQQQQ₅₀ QQQQPGQGPA₆₀
EPSQPSGPAV₇₀ ASLEPPVKLK₈₀ ELHFSNMKTV₉₀
DCVERKGKYM₁₀₀ YFTVVMAEGK₁₁₀ EIDFRCPQDQ₁₂₀
GWNAEITLQM₁₃₀ VQY

The sequence variation in both human isoforms and between mammalian species flanks the poly(glutamine) element and, the length of that homopolymeric sequence is also variable.

To specifically interfere with the activity of the PH domain of PHLDA1, two competitor peptide sequences were designed and synthesized (MPX741, MPX742 and MPX743). The design of competitor peptides was based on sequences upstream (i.e. N-terminal) and downstream (i.e. C-terminal) to the poly(glutamine) repeat element. MPX741 corresponds to a sequence N-terminal to the poly(glutamine) element (the predicted binding site for phosphatidylinositol lipids), MPX742 corresponds to a sequence approximately in the centre of the PHLDA1 PH domain, while MPX743 corresponds to C-terminal sequence to the poly(glutamine) element. The peptides were synthesised in the form of N-acetyl α-carboxamides to suppress terminal ionisation and degradation by exopeptidases. The peptides are detailed below.

| | |
|---|---|
| MPX741 | |
| KRSDGLLQLWKKKCCILTEEGLLLIPPK | (SEQ ID NO: 11) |
| MPX742 | |
| LEPPVKLKELHFSNMKTVD | (SEQ ID NO: 12) |
| MPX743 | |
| PQDQGWNAEITLQMVQY | (SEQ ID NO: 13) |

MPX741 corresponds to residues 9 to 36 of the PH domain set forth in SEQ ID NO: 11 and differs by 15 of its constituent amino acids from the PH domain of the other PHLDA family members. MPX742 corresponds to residues 73 to 91 of the PH domain set forth in SEQ ID NO: 12 and shares only 4 of its constituent amino acids with the PH domain of the other PHLDA family members. The N-terminal 5 amino acids of MPX742 correspond to the C-terminus of the unique PHLDA1 loop insertion (which is unique to the PH domain of PHLDA1). MPX743 corresponds to residues 117 to 130 of the PH domain set forth in SEQ ID NO: 13 and differs by 11 of its constituent amino acids from the PH domain of the other PHLD A family members.

The peptides were synthesised chemically at the Australian National University Australian Cancer Research Foundation Biomolecular Resource Facility. Solid-phase synthesis on Rink resin, 4-(2′,4′-dimethoxyphenyl-Fmoc-aminoethyl)-phenoxy polystyrene, was performed on a Symphony Peptide Synthesiser (Rainin Instrument Company Oakland Calif.) using standard Fmoc (9-fluorenylmethyloxycarbonyl) protocols and purified by reverse-phase HPLC. The identity of the purified products was confirmed by mass spectrometry.

### (ii) RBL-2H3 Degranulation Assay

Mast cell degranulation was measured using the β-hexosaminidase release method. RBL-2H3 cells were grown in F15 medium supplemented with 10% of fetal calf serum (FCS) and antibiotics. Cells were carefully detached from the culture flask with a cell scraper and plated at a concentration of 5×10⁵ cells/well in a 96 well microtiter plate and incubated in the presence of MPX741 peptide or MPX742 peptide overnight. RBL-2H3 cells were then incubated with 500 ng/ml anti-DNP IgE mb (SIGMA) for 2 hours at 37° C. After washing, cells were stimulated with 100 ng/ml DNP/albumin for 30 minutes. For non IgE specific stimulation 1 nM Ionomycin only was added to the cultures for 30 minutes. Supernatants were then collected and cell lysates were prepared using 0.1% Triton X-100. Lysate and supernatant samples (100) were transferred into 96-well plates and 50 μl 1 mM p-nitrophenyl-N-acetyl-β-D-glucopyranoside in 50 mM citrate buffer was added to each well and incubated for 1 hour at 37° C. in the dark. The reaction was stopped by adding 100 μl 0.1M NaHCO₃/0.1M Na₂CO₃ into each well and the absorbance measured at 405 nm. Percentage degranulation was calculated by the following formula:

OD supernatant/(OD supernatant+OD lysate)×100.

### Results

### (i) MPX741 and MPX742 Neutralises Mast Cell Activation

MPX743 (SEQ ID NO: 13) was observed to be poorly soluble and thus requires modification prior to further testing. MPX741 (SEQ ID NO: 11) and MPX742 (SEQ ID NO: 12) were demonstrated to to significantly reduced degranulation following IgE receptor crosslink (FIG. 11). Thus, treatment of RBL-2H3 cells with either MPX741 or MPX742 significantly inhibited mast cell activation.

### Example 8

### Additional NEDD9 SH3 Domain Competitor Peptides

Peptide MDD1 is demonstrated herein to be an inhibitor of mast cell activation, and hence administration of the peptide provides a means of treating and/or preventing hypersensitivity reactions. As described above in Example 6 above, MDD1 is a competitor peptide corresponding to residues 3-22 of the human sequence of the NEDD9 SH3 domain (shown in FIG. 5).

It is envisaged that variants produced by modification of one or more amino acid residues in the MDD1 peptide will in many cases emulate or improve one or more properties of the peptide such as, for example, the ability to desensitise mast cells, solubility, chemical and biochemical stability, cellular uptake, toxicity, immunogenicity and excretion of degradation products. Variants of the MDD1 peptide with similar or improved properties were designed by identifying particular amino acid residue/s in the MDD1 peptide sequence that may be either negative or positive determinants for a particular properties of interest. The functional activity of those MDD1 peptide variants will be tested using methods similar to those described in Example 6 above, and other methods generally known in the art.

For example, side-chain amputation will be utilised to substitute amino acids one at a time with alanine, along the sequence of the MDD1 peptide (as described in, for example, Gautam et al. 1995, “Binding of an invariant-chain peptide, CLIP, to I-A major histocompatibility complex class II molecules” Proc Natl Acad Sci USA, 92: 335-9). MDD1 peptide variants to be tested by this method include those set forth in SEQ ID NOs 33-49. Further, alanine will be substituted at multiple residues along the MDD1 peptide, and in particular at positions where negatively-charged residues exist (see SEQ ID NOs: 24-26).

It is also envisaged that increasing or reducing the length of the MDD1 peptide will also in many cases emulate or improve one or more properties of the peptide. Accordingly MDD1 variants of reduced length will be tested for their effects on mast cell activation and hypersensitivity using methods described herein and/or other methods generally known in the art. Exemplary sequences of such MDD1 peptide variants are set forth in SEQ ID NOs: 18-19 and 122-144).

Additional MDD1 peptide variants were designed and will be tested and these include those in which the cysteine residue at position 10 of the MDD1 peptide sequence is substituted with a serine (SEQ ID NO: 16) or an alanine (SEQ ID NO: 17). Length variants of the MDD1 peptide comprising a serine or alanine substitution at residue 10 will also be tested (i.e. length variants of SEQ ID NO: 16 and SEQ ID NO: 17) using the sequences set forth in SEQ ID NOs: 20-23 and 145-184. In addition, a further series of MDD1 variant peptides will be tested with the cysteine residue at position 10 substituted (as in SEQ ID NO: 16 or SEQ ID NO:17) along with single/multiple alanine substitutions at other residue/s along the chain, as per the sequences set forth in SEQ ID NOs: 90-121.

It is also envisaged that competitor peptides corresponding the full NEDD9 SH3 is domain (SEQ ID NO: 7) and other specific regions in the NEDD9 SH3 domain will be capable of modulating mast cell activation and thereby influence hypersensitivity reactions. A series of overlapping 20-mer peptides (SEQ ID NOs 14, 15 and 185-225) and 12-mer peptides (SEQ ID NOs: 226-273) will be tested using methods similar to those described in Example 6 above and/or other additional methods generally known in the art. Variants of those peptides with single/multiple alanine substitutions along the chain including variants with sequences set forth in SEQ ID NOs: 50-89).

Exemplary peptide sequences corresponding to at least a portion of the NEDD9 SH3 domain (and variants thereof) that will be synthesised and tested for effects on mast cell activation/influence on hypersensitivity are set forth in SEQ ID NOs: 14-273.

### Example 9

### Additional Competitor Peptides Derived on the NEDD9 SH3 Domain

Peptides MPX741 and MPX742 are demonstrated herein to be inhibitors of mast cell activation, and hence administering these peptides provides a means of treating and/or preventing hypersensitivity reactions. As described above in Example 7 above, MPX741 and MPX742 are competitor peptides corresponding to residues 9-36 and residues 73-91 respectively of the PH domain the human sequence of the PHLDA1 PH domain.

It is envisaged that variants produced by modification of one or more amino acid residues in the either of the MPX741 and MPX742 peptides will in many cases emulate or improve one or more properties of the peptide such as, for example, the ability to desensitise mast cells, solubility, chemical and biochemical stability, cellular uptake, toxicity, immunogenicity and excretion of degradation products. Variants of the MPX741 and MPX742 peptides with similar or improved properties were designed by identifying particular amino acid residue/s in their sequence that may be either negative or positive determinants for a particular properties of interest. The functional activity of such MPX741 and MPX742 peptide variants will be tested using methods similar to those described in Example 6 above, and methods generally known in the art.

For example, side-chain amputation will be utilised to substitute amino acids one at a time with alanine along the sequence of the MPX741 and MPX742 peptides (as described in, for example, Gautam et al. 1995, “Binding of an invariant-chain peptide, CLIP, to I-A major histocompatibility complex class II molecules” Proc Natl Acad Sci USA, 92: 335-9). MPX741 and MPX742 peptide variants to be tested by this method include those set forth in SEQ ID NOs 274-320. Further, alanine will be substituted at multiple residues along the MPX741 and MPX742 peptides, and in particular at positions where negatively-charged residues exist.

It is also envisaged that increasing or reducing the length of the MPX741 and MPX742 peptides will also in many cases emulate or improve one or more properties of the peptide. Accordingly MPX741 and MPX742 variants of reduced length will be tested for their effects on mast cell activation and hypersensitivity using methods described herein and/or other methods generally known in the art. Exemplary sequences of such MDD1 peptide variants are set forth in SEQ ID NOs: 321-374.

It is also envisaged that competitor peptides corresponding to the full PHLDA1 PH domain (SEQ ID NO: 10) and other specific regions of the PHLDA1 PH domain will be capable of modulating mast cell activation and thereby influence hypersensitivity reactions. A series of overlapping 20-mer peptides (SEQ ID NOs 14, 15 and 185-225) and 12-mer peptides (SEQ ID NOs: 375-488) will be tested using methods similar to those described in Example 6 above and/or other additional methods generally known in the art. Variants of those peptides with single/multiple alanine substitutions along the chain will also be tested.

Exemplary peptide sequences (and variants thereof) corresponding to the PHLDA1 PH domain that will be synthesised and tested for their effect on mast cell activation/hypersensitivity are set forth in SEQ ID NOs:274-488.

### Example 10

### Compositions

### (i) Injectable Parenteral Composition

A pharmaceutical composition of this invention in a form suitable for administration by injection may be prepared by mixing 0.005 mg to 5 g of one more suitable agents or compounds of the invention in 10% by volume propylene glycol and water.

### (ii) Composition for Oral Administration

A composition of one more suitable agents or compounds of this invention of the invention in the form of a capsule may be prepared by filling a standard two-piece hard gelatin capsule with 0.005 mg to 5 g of the agent or compound, in powdered form, 100 mg of lactose, 35 mg of talc and 10 mg of magnesium stearate.

(iii) Composition for Inhalation Administration

For an aerosol container with a capacity of 20-30 mL: a mixture of 0.005 mg to 5 g of one more suitable agents or compounds of the invention, 0.5-0.8% by weight of a lubricating agent, such as polysorbate 85 or oleic acid, is dispersed in a propellant, such as freon, and put into an appropriate aerosol container for either intranasal or oral inhalation administration.

1. A polypeptide for modulating mast cell activation, said polypeptide comprising an amino acid sequence from:
(i) a NEDD 9 Src homology 2 (SH2) domain,
(ii) a NEDD 9 Src homology 3 (SH3) domain, or
(iii) a PHLDA1 pleckstrin homology (PH) domain.

2. The polypeptide according to claim 1, wherein said polypeptide comprises an amino acid sequence as set forth in SEQ ID NO: 16.

3. The polypeptide according to claim 1, wherein said polypeptide consists of an amino acid sequence as set forth in any one of SEQ ID NOs: 14-15, 18-19, 122-144, or 185-273.

4. The polypeptide according to claim 1, wherein said polypeptide comprises an amino acid sequence as set forth in any one of SEQ ID NOs: 16-17, 20-121, or 145-184.

5. The polypeptide according to claim 1, wherein said polypeptide consists of an amino acid sequence as set forth in SEQ ID NO: 8.

6. The polypeptide according to claim 1, wherein said polypeptide comprises an amino acid sequence as set forth in SEQ ID NO: 10.

7. (canceled)

8. The polypeptide according to claim 1, wherein said polypeptide comprises an amino acid sequence as set forth in any one of SEQ ID NOs: 274-319.

9. The polypeptide according to claim 1, wherein said polypeptide consists of an amino acid sequence as set forth in SEQ ID NO: 10, SEQ ID NO: 11 or SEQ ID NO: 12.

10-14. (canceled)

15. A method of inhibiting or preventing mast cell activation in a subject, the method comprising administering to the subject a therapeutically effective amount of a polypeptide comprising an amino acid sequence from:
(i) a NEDD 9 Src homology 2 (SH2) domain,
(ii) a NEDD9 Src homology 3 (SH3) domain, or
(iii) a PHLDA1 pleckstrin homology (PH) domain.

16-19. (canceled)

20. A method for treating an allergic disease or condition in a subject, the method comprising administering to the subject a therapeutically effective amount of a polypeptide comprising an amino acid sequence from:
(i) a NEDD 9 Src homology 2 (SH2) domain,
(ii) a NEDD9 Src homology 3 (SH3) domain, or
(iii) a PHLDA1 pleckstrin homology (PH) domain.

21. The method according to claim 20, wherein the allergic disease or condition is selected from the group consisting of anaphylaxis, allergic drug reactions, skin allergy, eczema, allergic rhinitis, urticaria, atopic dermatitis, contact allergy, food allergy, allergic conjunctivitis, insect venom allergy and allergic respiratory diseases or disorders.

22. The method according to claim 21, wherein the allergic respiratory disease or disorder is allergic asthma or allergic rhinitis.

23. The method according to claim 20, wherein said polypeptide comprises an amino acid sequence as set forth in SEQ ID NO: 7.

24. The method according to claim 20, wherein said polypeptide comprises an amino acid sequence as set forth in any one of SEQ ID NOs: 8 or 14-273.

25. (canceled)

26. The method according to claim 20, wherein said polypeptide consists of an amino acid sequence as set forth in SEQ ID NO: 8, or any one of SEQ ID NOs: 14-16.

27. The method according to claim 20, wherein said polypeptide comprises an amino acid sequence as set forth in SEQ ID NOS: 10-12, or any one of SEQ ID NOs: 274-348.

28-48. (canceled)

49. The method according to claim 15, wherein said polypeptide comprises an amino acid sequence as set forth in SEQ ID NO: 7.

50. The method according to claim 15, wherein said polypeptide comprises an amino acid sequence as set forth in SEQ ID NO: 8: or any one of SEQ ID NOs: 14-273.

51. The method according to claim 15, wherein said polypeptide consists of an amino acid sequence as set forth in SEQ ID NO: 8 or any one of SEQ ID NOs: 14-16.

52. The method according to claim 15, wherein said polypeptide comprises an amino acid sequence as set forth in any one of SEQ ID NOs: 10-12, or any one of SEQ ID NOs: 274-348.
